(19) United States
(12) Patent Application Publication
(10) Pub. No.: US 2024/0398860 A1
(43) Pub. Date: Dec. 5, 2024

(54) MECHANOCHEMICALLY PRE-TREATED, HEAVY-METAL-FREE ACTIVATED CARBON PARTICLES A, TOPICAL PHARMACEUTICALS, MEDICAL PRODUCTS AND COSMETICS, PREPARATION PROCESS AND USES

(71) Applicant: Smart Material Printing B.V., Enschede (NL)

(72) Inventor: Gregor LUTHE, Gronau (DE)

(21) Appl. No.: 18/696,842

(22) PCT Filed: Sep. 28, 2022
(86) PCT No.: PCT/EP2022/076924

§ 371 Date: Mar. 28, 2024

(30) Foreign Application Priority Data
Sep. 29, 2021 (DE) 102021004905.5
Sep. 29, 2021 (DE) 102021004906.3
Sep. 29, 2021 (DE) 102021004907.1

(51) Int. Cl. A61K 33/44; A61K 8/19; A61K 9/00; A61K 9/06; A61K 9/14; A61P 17/00; A61P 39/00; A61Q 19/00; C01B 32/384

(52) U.S. Cl. CPC A61K 33/44; A61K 8/19; A61K 9/0014; A61K 9/06; A61K 9/14; A61P 17/00; A61P 39/00; A61Q 19/00; C01B 32/384; C01P 2004/62; C01P 2004/64; C01P 2006/12; C01P 2006/80

(57) ABSTRACT

Activated carbon particles A which are mechanochemically pre-treated under at least one inert gas, according to X-ray emission spectroscopy, are free of heavy metals and free of persistent organic pollutants (POP), polycyclic aromatic hydrocarbons (PAH), fibrous fractions and meso- and macropores, have a carbon content >99 mol % and a particle size determined by electron microscopy in the range from 50 nm to 1000 nm or from 100 nm to 1000 nm, a mechanochemical process for their preparation and their use for the preparation of decorative and medical cosmetics and care products, medical products and pharmaceuticals, topical pharmaceuticals, medical products and cosmetics, preparation processes therefor and corresponding uses.

### FIELD OF THE INVENTION

The present invention relates to mechanochemically pre-treated, heavy metal-free activated carbon particles A, in particular activated carbon particles A which are, according to X-ray emission spectroscopy, free of heavy metals and persistent organic pollutants (POP), polycyclic aromatic hydrocarbons (PAH), fibrous fractions and mesopores and macropores and have a particle size determined by electron microscopy in the range from 50 nm to 1000 nm, or in other variants from 100 nm to 1000 nm, and, inter alia, for use in medicine.

The present invention also relates to a mechanochemical process for producing the activated carbon particles A.

Furthermore, the present invention relates to heavy metal-free topical pharmaceuticals, medical products and cosmetics, inter alia for use in medicine, containing mechanochemically pre-treated, heavy metal-free activated carbon particles A.

The present invention also relates to a process for the preparation of heavy metal-free topical pharmaceuticals, medical products and cosmetics containing mechanochemically pre-treated, heavy metal-free activated carbon particles A.

Furthermore, the present invention relates to the use of the activated carbon particles A for the preparation of cosmetics, personal care products, care products, products for saunas and steam baths, powders, solids, gels, creams, ointments, lotions, drops, sprays, metered dose aerosols, gargle solutions, lozenges, enemas, clysters, foams, coatings on textile fabrics, face masks, gauze, plasters, medical products and components of release materials.

Furthermore, the present invention relates to the use of the heavy metal-free topical pharmaceuticals, medical products and cosmetics in the form of powders, solids, gels, creams, ointments, lotions, drops, sprays, metered dose aerosols, gargle solutions, lozenges, enemas, clysters, foams, coatings and coating materials on and/or in release materials, sponges, textile fabrics, face masks, gauze, plasters, roll-on sticks and medical devices.

Last but not least, the present invention relates to heavy-metal-free topical pharmaceuticals, medical products and cosmetics containing mechanochemically pre-treated, heavy-metal-free activated carbon particles A strikingly prepared in the form of powders, solids, gels, creams, ointments, lotions, drops, sprays, metered dose aerosols, gargle solutions, lozenges, enemas, clysters, foams, release materials, coatings and coating materials on and/or in release materials, sponges, textile fabrics, face masks, gauze and plasters as well as on and/or in roll-on sticks, dispensers and medical devices.

### STATE OF THE ART

In medicine, topical pharmaceuticals, medical products and cosmetics and care products are understood to be all those drugs that are applied locally rather than being ingested or injected. Since topical application brings the active ingredient directly to the site of action, the dose can often be reduced as far as possible, thus minimising the risk of side effects. All organs on the surface, i.e. the skin and the mucous membranes of the respiratory tract, the digestive tract and the external eye, are accessible to local treatment or care. However, an injection into a joint is also a topical (local, intra-articular) treatment.

Topical medicines and care products play a particularly important role in dermatology, as well as in ophthalmology (eye medicine) and urology.

Applications include, inter alia: on the skin: ointments, creams and lotions; on the nose: nasal drops, sprays and ointments; on the oral mucosa: Gargle solutions and lozenges; on the respiratory tract: Metered dose aerosols, nebulisation with UV light and powder inhalers; on the intestines: enemas and clysters; on the conjunctiva of the eyes: drops, ointments and sprays.

Antibiotics can also be used topically, for example for bacterial skin infections. Sulphonamides are mainly used here. In contrast to medical products and cosmetics, these drugs have a pharmacological, metabolic and/or immunological effect.

Among other things, the drugs are used to combat unpleasant smelling, painful, itchy, corrosive, unaesthetic and inflammatory external, i.e. topical, diseases. They are also used for the topical treatment of injuries and burns.

Medical products and cosmetics include substances or medical products and cosmetics made from substances that are used for therapeutic or diagnostic purposes, whereby the intended effect, in contrast to pharmaceuticals, is not primarily pharmacological, metabolic or immunological, but mostly physical or physical or physical-chemical. Medical products serve in particular to support the therapeutic effect of pharmaceuticals by combating, among other things, unpleasantly smelling, painful, itchy, corrosive or unaesthetic external, i.e. topical, side effects of diseases and injuries.

Cosmetic cleansing products for the skin and/or hair are known from the German utility model DE 20 2014 010 286 U1, which contain surfactants, preferably anionic and amphoteric surfactants, activated carbon in the form of activated carbon particles with a particle size of 0, 5 to 60 μm, pearlescent agents selected from the group of ethylene glycol monostearates, polyethylene glycol distearates and glycol distearates as well as at least one structurant from the group of acrylate copolymers and alkoxylated thickeners. The activated carbon used is CI 77266 Carbon Black. No further information is provided on the activated carbon. Its use as a pharmaceutical is also not described.

From the German patent application DE 10 2017 010 930 A1 and the corresponding international patent application WO 2019/101357 A1, topical medical products are known which contain at least one type of carbon particles with an average particle size of d₅₀=650±200 nm. These topical medical products can be used for the adsorption and/or decomposition of chemotherapeutic agents that have been excreted via the skin, as sauna paste detoxification of the body, as a peeling paste, as a fat-free moisturising and cleansing cream, as a protective product for nursing staff who come into contact with chemotherapeutic agents, as a shower gel, as an ointment for the accompanying treatment of calcinosis cutis and as a lotion for the care of the scalp and hair roots. In addition, the test reports in the file show that the topical medical products quickly relieve the severe itching after contact of the human skin with oak processionary moths and the painful swelling of joints affected by arthritis.

The known topical medicinal products contain the activated carbon particles in an amount of 0.01% to 10% by weight, based on the respective total amount of a topical preparation.

In order to increase the effect of the topical pharmaceuticals and thereby reduce the amount of pharmaceuticals to be applied, it would be desirable to set the content of activated carbon particles in the topical pharmaceuticals particularly high. However, the known topical medicinal products containing more than 10% by weight of activated carbon particles tend to segregate, which makes larger quantities and stabilisers necessary. However, this changes the application-technical property profile unfavourably in an unpredictable way.

Nowadays there are many different care products. By the term care products, for example, products for hair care, skin care, oral care and/or nail care are understood. Care products for the sauna are a very good example. In the Turkish sauna, also known as the hamam, a sauna product is used which is applied to the skin and contains black amber, among other things. In addition, salt is often rubbed into the skin before a sauna session. This should help the body with the cleansing.

In the following list the individual ingredients that are known to be contained in the individual product groups are listed:

As base ingredients emulsifiers, film formers, gelling agents, solvents, oil components, soap base, surfactants, thickening regulators and/or consistency regulators, waxes.

For hair care anti-dandruff agents, hair bleaching agents and/or bleach boosters, hair dyes, hair-setting polymers, hair care substances and/or conditioning agents.

For skin care antibacterial agents, antioxidants, antiperspirant agents and/or astringents, deodorising agents, skin bleaching agents, cooling agents, moisturisers and/or moisturising agents, cleansing agents, abrasive agents and/or polishing agents, moisturising agents, self-tanning agents, sun protection and/or UV filters, active ingredients for skin care, active ingredients for chemical hair removal and/or depilation.

For oral care antibacterial agents, bleaching agents for teeth, cooling agents, cleaning agents, abrasive agents and/or polishing agents, agents against sensitive teeth, agents against dental plaque and/or caries.

For nail care: nail hardener, nail serum.

As aids alkalising agents, acids and/or neutralising agents, antioxidants, binding agents, dyes/colour pigments, humectants, resins, preservatives, oxidising agents, reducing agents, stabilisers, propellants for sprays, opacifiers/pearlescent agents, denaturants, plasticisers, flavours and/or fragrances, essential oils, flavours, perfumes/fragrances.

The fields of application decorative cosmetics, facial care, hair care, body care, sun protection, dental and oral care.

A mechanochemical process for processing natural, synthetic, biogenic and biological materials is known from the German patent application DE 10 2019 006 084 A1. For example, the mechanochemical immobilisation of carcinogenic hexavalent chromium is described therein; the strong binding of chromium in the activated carbon is a valuable advantage, but it is a disadvantage in the context of the present invention.

In the German patent application DE 10 2018 000 418 A1 a mechanochemical process for the preparation of valuable products free of persistent organic pollutants and other organohalogen compounds is described.

From the German patent application DE 102 61 204 A1 a process for the decontamination or detoxification of solid or liquid products contaminated with environmental toxins such as polyhalogenated compounds or organochlorine substances by means of high-kinetic processes in which the contaminated products are subjected to high-kinetic fine comminution using so-called tribomaterials by the multiple effects of impact or shear forces is known. It must be assumed that activated carbon is contaminated with heavy metals during the grinding process.

A topical composition is known from the international patent application WO 2019/101357 A1, which contains at least one type of carbon particle with an average particle size of d₅₀=650±200 nm. There is no information on the heavy metal content. In contrast to the technical teaching of the international patent application, according to which a phosphorus content is a valuable advantage, a high phosphorus content is a disadvantage in the context of the present invention.

In the publication by D. V. Onishchenko, “Mechanochemical Treatment of Amorphous Carbon from Brown Sphagnum Moss for the Preparation of Carbon Nanotubes” in Surface Engineering and Applied Electrochemistry, 2013, Volume 49, No. 6, pages 445-449, the preparation of multi-walled carbon nanotubes from the dried sphagnum moss is described. The end product has a specific surface area of 2305 m²/g shortly after treatment, which decreases to 1050 m²/g after prolonged storage.

### OBJECT OF THE PRESENT INVENTION

The present invention was based on the object of further developing the activated carbon particles of the international patent application WO 2019/101357 A1 in order to prepare new activated carbon particles with novel properties and particularly broad applicability. In addition, a new method was to be found with which these new activated carbon particles could be prepared in a targeted manner.

Furthermore, the present invention was based on the object of providing topical pharmaceuticals for use in medicine.

The topical pharmaceuticals should strikingly be prepared in the form of powders, solids, gels, creams, ointments, lotions, drops, sprays, metered dose aerosols, gargle solutions, lozenges, enemas, clysters, foams, release materials, coatings and coating materials on and/or in release materials, sponges, textile fabrics, face masks, gauze and plasters as well as on and/or in roll-on sticks, dispensers and medical devices.

The topical pharmaceuticals should be particularly suitable for the therapeutic treatment of inflammatory diseases and infections, wounds, burns, chemical burns, lesions, itching, burning, intertrigo, infections and discolouration and pain (i) in and on the skin, in and on mucous membranes, in particular in and on mucous membranes in the mouth, and in and on genitalia, (ii) in the intestine, (iii) in and on eyes, ears, mouth, lips, fingernails, toenails, cornea, warts and scars, (iv) for healing the consequences of excretion of noxae, drugs, toxins, medicaments, acids and bases via the skin and (vi) for the preventive protection of humans and animals.

The present invention was also based on the object of providing topical medical products and cosmetics, in particular in the form of powders, solids, gels, creams, ointments, lotions, drops, sprays, metered dose aerosols, gargle solutions, lozenges, enemas, clysters, foams, coatings and coating materials on and/or in release materials, sponges, textile fabrics, face masks, gauze, plasters, roll-on sticks and medical devices.

The topical medical products and cosmetics should be particularly useable with advantage as oncological and dermatological medical products and cosmetics as well as for the support of healing processes (i) in and on the skin, in and on mucous membranes, in particular in and on mucous membranes in the mouth, and in and on genitals, (ii) in the intestine, (iii) in and on eyes, ears, mouth, lips, fingernails, toenails, cornea, warts and scars, (iv) for excretion of noxae, drugs, toxins, medicaments, acids and bases via the skin, (v) for lesions, itching, burning, intertrigo, infections and redness, (vi) for preventive protection, (vii) for existing infections, wounds or burns in humans and animals.

Furthermore, the above-mentioned pharmaceuticals, medical products, cosmetics and other products should not harm the environment and, in particular, should not accumulate in organisms in the marine food chain.

It is also the object of the present invention to find a method for preparing the above-mentioned pharmaceuticals, medical products, cosmetics and other products.

Further objects arise for the skilled person on the basis of the following description and disclosure, as well as the claims.

### Solution According to the Invention

These objects have been solved by the subject matter shown in the independent claims.

Special and preferred embodiments can be found in the dependent claims and the following description.

### Advantages of the Invention

In view of the state of the art, it is surprising and not foreseeable for the skilled person that the objects on which the present invention was based are solved by the subject matters disclosed in the independent claims as well as the further subject matter disclosed below.

In particular, it was surprising that the activated carbon A according to the invention did not exhibit mesoporous and macroporous and yet were highly effective in the applications and uses according to the invention. As they were free of heavy metals, there was also no risk of allergic reactions to heavy metals such as nickel or chromium. A particular advantage of the method according to the invention for preparing the activated carbon particles A according to the invention was that the ubiquitous POPs and PAHs were degraded to carbon and became a component of the activated carbon particles A according to the invention.

In particular, it was surprising that the heavy metal-free topical medical products and cosmetics according to the invention can be used excellently for the care of overlappings of skin on the chest, abdomen and groin, for the supportive therapy of mucous membranes, eyes, ears, mouth, lips, fingernails, toenails, cornea, warts and scars, for the removal and/or neutralisation of excretiona of noxae, drugs, toxins, medicines, acids, bases and odorous substances via the skin, for the alleviation of unpleasant and/or painful accompanying symptoms and side effects of lesions, itching, intertrigo, burning, infections, in the cancer therapy, the chemotherapy, for the supportive therapy for chondrocalcinosis, psoriasis, neurodermatitis, acne, with eczema and atrophic eczema, warts, rosacea, herpes, shingles, measles, mumps, rubella, chickenpox, hand-mouth-foot diseases, hand and foot syndromes, redness, rheumatism and arthrosis as well as the preventive protection and support of healing processes in existing infections, wounds or burns in humans and animals.

The topical pharmaceutics for use in human and veterinary medicine are particularly suitable for the therapeutic treatment of inflammatory diseases and infections, wounds, burns, chemical burns, lesions, itching, burning, intertrigo, infections and discolouration and pain (i) in and on the skin, in and on mucous membranes, especially in and on mucous membranes in the mouth, and in and on genitalia, (ii) in the intestines, (iii) in and on eyes, ears, mouth, lips, fingernails, toenails, cornea, warts and scars, (iv) to heal the consequences of excretion of noxae, drugs, toxins, medicaments, acids and bases via the skin and (vi) for the preventive protection of humans and animals.

Furthermore, the topical pharmaceutics do not harm the environment and, in particular, they do not accumulate in organisms in the marine food chain.

Furthermore, the topical pharmaceuticals, medical products and cosmetics according to the invention can be prepared in a simple and highly reproducible manner using the method according to the invention.

It is particularly surprising that during the preparation of the topical pharmaceuticals, medical products and cosmetics according to the invention largely or exclusively renewable, compostable, toxicologically harmless, biodegradable and/or recyclable raw materials are used, so that the preparation and use of the topical pharmaceuticals, medical products and cosmetics according to the invention are particularly environmentally friendly and act as a carbon dioxide sink.

Particularly surprising is the binding and/or the assimilation of pollutants from the skin by the topical pharmaceuticals, medical products and cosmetics according to the invention. In particular, drugs secreted through the skin and mucous membranes, especially cytostatic drugs, are prevented from re-absorbtion and rendered harmless. The topical pharmaceuticals, medical products and cosmetics according to the invention are thus tolerable by allergy sufferers and/or all skin types. In addition, they can be easily adjusted to be bacteriostatic, antiviral, fungicidal or fungistatic and nematicidal.

All these favourable effects of the topical pharmaceuticals, medical products and cosmetics according to the invention are enhanced by their excellent penetration depth into the skin and mucous membranes. They can therefore also serve as carriers for other medicaments.

### DETAILED DESCRIPTION OF THE INVENTION

In the context of the present invention, the term “mesoporous” is used in the sense of the IUPAC definition, i.e. it means pore sizes between 2 nm and 50 nm.

Accordingly, in the context of the present invention, the term “macroporous” in the sense of the IUPAC definition means pore sizes of >50 nm.

In the context of the present invention, the term “coarsely particulate” preferably means a particle size of 0.5 cm to 5 cm and in particular 1 cm to 3 cm.

In one aspect, the present invention is directed to activated carbon particles A which are mechanochemically pre-treated under at least one inert gas and which, according to X-ray emission spectroscopy, are free of heavy metals as well as persistent organic pollutants (POPs), polycyclic aromatic hydrocarbons (PAHs), fibrous fractions and meso- and macropores, have a carbon content >99 mol %, preferably >99.2 mol %, a particle size determined by electron microscopy in the range from 50 nm to 1000 nm or in other variants from 100 nm to 1000 nm, preferably 200 nm to 900 nm, preferably 300 nm to 800 nm and in particular 400 to 700 nm.

The (topical) pharmaceuticals, medical products and cosmetics according to the invention contain, activated carbon particles A which are pre-treated mechanochemically under at least one inert gas and are, according to X-ray emission spectroscopy, free from heavy metals as well as persistent organic pollutants (POPs), polycyclic aromatic hydrocarbons (PAHs), fibrous fractions and meso- and macropores, have a carbon content >99 mol %, preferably >99.2 mol %, a particle size determined by electron microscopy in the range from 50 nm to 1000 nm or in other variants from 100 nm to 1000 nm, preferably 200 nm to 900 nm, preferably 300 nm to 800 nm and in particular 400 to 700 nm.

Preferably, the at least one inert gas is selected from the group consisting of nitrogen, helium, neon, argon and xenon. The inert gases, in particular the nitrogen, can also be solid and/or liquid. Liquid nitrogen is particularly advantageous because its low surface tension finely disperses the material to be ground particularly quickly. The inert gases can also be present as solids, which has the advantage that they act as additional grinding media.

The absence of mesopores and macropores is verified using electron micrographs.

The electron micrographs also show that the activated carbon particles A according to the invention all have a granular structure and do not have fibrous structures such as those described in the publication by D. V. Onishchenko, “Mechanochemical Treatment of Amorphous Carbon from Brown Sphagnum Moss for the Preparation of Carbon Nanotubes” in Surface Engineering and Applied Electrochemistry, 2013, Volume 49, No. 6, pages 445-449.

In the context of the present invention, the term “heavy metals” includes strontium, barium, scandium, yttrium, lanthanum and the lanthanides, the actinides, titanium, zirconium, hafnium, chromium, molybdenum, tungsten, manganese, technetium, rhenium, iron, ruthenium, osmium, nickel, palladium, platinum, copper, silver, gold, zinc, cadmium, mercury, gallium, indium, thallium, germanium, tin, lead, arsenic, antimony, bismuth, selenium and tellurium as well as the compounds of these metals.

In the context of the present invention, the property “free of heavy metals” means that the concentrations of the respective heavy metals are below the maximum limits permitted under food regulatory and medical law.

Polycyclic aromatic hydrocarbons PAHs are in particular naphthalene, acenaphthylene, acenaphthene, fluorene, phenanthrene, anthracene, fluoranthene, pyrene, benzo[a]anthracene, chrysene, benzo[b]fluoranthene, benzo[k]fluoranthene, benzo[a]pyrene, dibenzo[a,h]anthracene, indeno[1,2,3-cd]pyrene, benzo[ghi]perylene.

The persistent organic pollutants (POPs) are mainly halogenated organic compounds that accumulate in the food chain. All substances included in the Stockholm Convention belong to this group. The so-called Dirty Dozen consists only of organochlorine compounds. Organochlorine pesticides such as DDT or endrin are of particular importance. In addition, there are brominated flame retardants and polyfluorinated compounds such as perfluorooctane sulfonates (PFOS), which are used as surface-active substances in textiles.

The concentrations of PAHs and POPs are generally measured using GC-MS coupling. The activated carbon particles A according to the invention are considered to be free of PAHs and POPs if they cannot be detected using this method.

The activated carbon particles A according to the invention are produced using the mechanochemical process according to the invention from the coarsely particulate activated carbon particles as material to be ground F. Preferably, the coarsely particulate activated carbon particles have a particle size of 0.5 cm to 5 cm and in particular 1 cm to 3 cm.

Starting materials for the coarsely particulate activated carbon particles are charcoal and/or its sieve residues and/or wood ash, activated carbon, hard coal, animal coal, animal waste coal, pyrogenic carbon of varying degrees of pyrolysis, functionalised carbon and/or functionalised coal, pre-treated coal, washed coal, coal with varying degrees of carbonisation, extracted coal, coke, natural coke and incompletely gasified coke and/or the starting materials are obtained by a pyrolysis process in a mobile or stationary pyrolysis plant from predominantly lignin-rich, organic materials such as wood, plant stems, leaves, fruit stones, nutshells and/or bones.

This is a significant difference to the known Onishchenko process, in which finely divided, carbonised peat moss is used.

Examples of suitable starting materials for pyrolysis, which is preferably carried out above 300° C. and preferably above 500° C., are cherry trees, bamboo, shrubs, beeches, oaks and ash as well as C4 plants that have a crown anatomy, in particular grasses, maize, sugar cane, millet, giant china reed and amaranth. The coarsely particulate activated carbon particles produced from these raw materials are also designated as biochars.

It is a particular advantage of the process according to the invention described below that during the grinding process any PAHs and POPs present are decomposed into carbon, which then becomes a component of the activated carbon particles A according to the invention.

A further particular advantage of the process according to the invention is that the organically bound halogens can be converted into halides if, for example, inorganic carbonates such as mussel shells are added. The activated carbon particles A according to the invention can then be very easily separated from the resulting inorganic halides via the difference in density.

The mechanochemical process according to the invention is carried out using mechanical mills. The usual and known mechanical mills can be used for the process according to the invention. Examples of suitable mechanical mills are ball mills, hammer mills, pinned disc mills, jet mills, vibration mills, shaking mills, horizontal mills, attritors and planetary mills. For example, the mechanical mill described in the German publication DE 195 04 540 A1, FIGS. 1a to 4b, can be used.

The means for agitating the grinding media in an energy-rich mass flow together with the material to be ground can, apart from the attritors according to the above-mentioned German patent application, have different shapes, which are preferably symmetrical with respect to the at least one, in particular one, drive shaft. Preferably, these agitation means are made of ceramics and metals and alloys coated with ceramics.

For example, the force transmission can occur by planar impact blades that have at least two impact ends that are arranged symmetrically to the drive shaft so that no imbalance occurs. The impact blades can be arranged on the drive shaft spaced and/or aligned and preferably have circular holes in the area of their ends.

However, planar impact discs can also be used, which preferably have a circular circumference and preferably have circular, oval, elliptical and/or elongated holes bent parallel to the circumference, which are preferably arranged in a circle and preferably at the same distance from each other. The impact discs can be arranged on the drive shaft in such a way that the holes are spaced and/or aligned.

However, the planar impact discs can also have raised webs on their surface, which are arranged symmetrically and run from the drive shaft to the circular edge in a straight line and/or preferably curved in the direction of rotation. The beginnings and ends of the webs are preferably arranged at the same distance from each other. Preferably, at least four of these webs are used. In a further embodiment, these webs are arranged on the two opposite sides of the planar impact discs. The webs can have a quadrangular, a triangular or a semi-circular cross-section. The impact discs can be arranged on the drive shaft in such a way that the webs are spaced and/or aligned.

However, the force transmission to the grinding media can also occur by impact fans arranged symmetrically to the drive shaft. These are formed by a ring that surrounds the drive shaft and at least two impact fans radiating from this ring. In a further embodiment, the ring widens on at least two surfaces arranged symmetrically to the drive shaft, each of which merges into at least two impact fans radiating outwards. Preferably, the edges are curved and lie on an imaginary circle around the entire arrangement. They can therefore be n-fold single impact fans or n-fold multiple impact fans, where n=at least 2.

In a further embodiment, the impact fans can have raised webs on one side or on two opposite sides, which are arranged symmetrically to each other and extend from the drive shaft to the respective edge of the impact fans. The webs can have triangular, quadrangular or semi-circular profiles.

In yet another embodiment, these webs can run parallel to the curved edges of the impact fans.

The agitation means described above preferably have a spacing of at least one width of a grinding media. The width of the agitation means, viewed transversely to the drive shaft, is preferably 0.1 to 8 times the width of the respective grinding media. The agitation means can be arranged on the drive shaft parallel to each other or offset.

Instead of the agitation means described above, impact lobes can also be used which are arranged symmetrically and on the drive shaft and which have impact bodies which are connected to the ring surrounding the drive shaft by rods of the same or different lengths, straight or curved. The impact bodies can be cuboidal, blade-shaped, spherical or drop-shaped bodies or ellipsoids. To prevent imbalance during rotation, they preferably have the same weight.

The drive shafts themselves can be splined shafts, hollow shafts, PTO shafts, worm shafts, tapered shafts, conical shafts or triangular shafts.

Instead of the arrangements of a drive shaft and the agitation means arranged around it as described above, at least two rollers arranged parallel to each other in the longitudinal direction of the grinding chamber and rotating against each other can be used. The grinding of the material to be ground takes place in the area where the rollers touch each other.

In one embodiment, the rollers are arranged at an angle to the longitudinal axis of the grinding chamber, which results in additional torsion of the material to be ground.

In yet another embodiment, the roller rotates against an abrasion surface so that grinding takes place in the area of contact between the roller and the abrasion surface.

In further embodiments, the surfaces of the rollers and the abrasion surfaces may have structures, such as teeth, nipples and/or depressions.

In yet another embodiment, the impact effect and the fit can be improved by a springing of the roller surface. This can be achieved by the rollers as a whole being spring-loaded against each other. However, individual points of the roller surface can also be spring-loaded separately. This can be achieved, for example, by the rollers having recesses in the area of their surface in which balls are arranged which are pressed out of the recesses by spiral springs. The recesses can have a larger clear width than the radius of the ball, so that the material to be ground that enters the recesses trickles out of the recesses again as the rollers continue to rotate.

The grinding chamber, in which the agitation means are arranged, is preferably tubular in shape and preferably has a circular circumference. If the rollers described above are used, the grinding chamber can also be defined by two opposing, parallel, straight walls which extend at a distance across the width of the rollers and which are connected by two opposing curved walls.

Viewed in the longitudinal direction, i.e. along the drive shaft, the circumference of the grinding chamber can constrict at least once, resulting in at least two spherical cut-shaped grinding chambers arranged one behind the other, which are connected to each other by a circular opening. The dimensions of the agitation means in the individual grinding chambers are then adapted to the course of the walls so that the agitation means are also largest at the largest diameter of the spherical cut-shaped grinding chambers, whereby their dimensions decrease to the left and to the right, following the rounding of the grinding chambers.

The mechanical mills can be driven directly by the energy supplied by wind turbines, water turbines and tidal power plants.

Repulsion motors have proven their worth for the shock-free start-up of large mechanical mills. These can then be throttled in their power output during subsequent continuous operation.

A combination of a powerful motor for start-up, which is switched off after start-up, and a weaker motor for continuous operation can also be used.

Gas turbines and combustion engines fuelled with fossil or biotechnologically produced fuels can also be used.

Asynchronous motors, DC motors (commutator motors), AC and three-phase motors, rotating field and travelling field machines, three-phase asynchronous machines, slip ring motors, three-phase synchronous machines, cascade machines, stepper motors, brushless DC motors, linear motors, AC motors, capacitor motors, shaded-pole motors, synchronous motors, single-phase asynchronous motors, reluctance motors, magnetic motors, transverse flux machines, current inverter or commutator machines, direct current motors, universal motors (for direct and alternating current), permanently excited direct current motors, electrically excited (separately excited) direct current motors, series motors, shunt-wound machines, compound motors, ball bearing motors, unipolar machines, homopolar motors and Barlow wheels are also suitable.

Preferably, electric motors according to the international patent application WO 2017/055246 A2 are used. These comprise at least one electric machine component with at least one winding for generating a magnetic field, which comprises at least one waveguide which has a casing and an inner cavity through which a coolant can be conducted, wherein the winding has two ends to which an electrical operating voltage is connected and wherein
the waveguides are round tube-shaped and have an outer diameter in the range of 3 mm,
the ends of the winding each serve as a coolant inlet or coolant outlet, and
the ends of the winding are connected to a connecting piece which comprises a coolant inlet and/or a coolant outlet, several waveguide connections for connecting waveguides, a distribution channel via which the coolant is fed into at least one waveguide and/or a collecting channel into which the coolant emerging from at least one waveguide flows and is guided to the coolant outlet of the connecting piece.

Electric motors of this type are marketed by Dynamic E Flow GmbH, Kaufbeuren, Germany, under the Capcooltech® brand. The HC and LC types are preferably used.

However, motors driven by compressed air can also be used, which are particularly suitable for use in explosion-proof areas.

Grinding can take place at a temperature of the grinding media and the material to be ground of −273° C. to +500° C.

The duration of grinding can vary widely and can therefore be perfectly adapted to the respective task. Preferably, the grinding time is 0.5 minutes to 1000 hours, preferably 10 minutes to 500 hours, particularly preferably 10 minutes to 100 hours and especially 10 minutes to 50 hours.

According to the invention, all surfaces inside the mills described above which come into contact with the material to be ground or with the material to be ground and the grinding aid are coated with at least one, in particular one, heavy-metal-free, inorganic, technical ceramic.

The ceramic can be an oxide ceramic and/or a non-oxide ceramic. Examples of suitable ceramics are aluminium oxide, boron carbide, boron nitride, boron nitride carbide, calcium silicate, silicon oxide, silicon carbide, silicon nitride, silicon oxide nitride, silicon oxide carbide, silicon nitride carbide, silicon oxide nitride carbide, glass, boron carbide, boron nitride, boron nitride carbide, silicon aluminium oxide nitride and glass ceramics.

In contrast to all other materials, ceramic products, in particular oxide ceramics, are first moulded from the raw materials and then (i.e. after moulding) transferred to the ceramic material in a high-temperature process or sintering process with the aim of material conversion to produce material-locking bonds between the raw material grains. In contrast to other materials, the raw materials therefore have two basic tasks: On the one hand, they must guarantee the chemical composition of the desired ceramic materials and, on the other hand, allow them to be moulded beforehand. The ceramic blank therefore has a significantly lower mechanical strength than a metallic blank, for example. This is why it is also known as a green body, which has nothing to do with its colour.

Regardless of the composition, the manufacture of ceramic products always involves the following steps:
1. preparation of the raw materials,
2. preparation of the ceramic body
3. moulding,
4. removal of aids such as water and/or organic additives for moulding,
5. if necessary, mechanical processing of the blanks or glazing,
6. ceramic firing as well as
7. various post-treatment and finishing processes including glazing or decorating and re-firing.

Thieme Römpp Online 2014, Version 3.45, “Keramik, Table 1: Formgebungsverfahren für tonkeramische Massen mit Produktionsbeispielen” provides an overview of the preparation of oxide ceramics. Examples of suitable oxide ceramics can be found in the German patent applications DE 196 28 820 A1, ScienceDaily®, “Novel Ceramic Foam is Safe and Effective Insulation”, 18 May 2001, the company publication “Promat High Perfomance Insulation, Theoretische Grundlagen der technischen Wärmedämmung”, or the article by F. Luthardt and Jörg Adler, “A Ceramic Foaming Technology for High-Temperature Insulation Materials”, Fraunhofer IKTS Annual Report 2012/13, pages 32 and 33.

Non-oxide ceramics do not contain oxygen. Instead, the anions are carbon, nitrogen, boron and silicon. An exception are a few mixed ceramics, which also contain some oxygen in addition to the anion mentioned, such as silicon aluminium oxide nitride. However, the cations also differ significantly from the oxide ceramics. When silicon ions occur as cations, the homeopolar bond prevails, so that one cannot actually speak of cation and anion in exact chemical terms.

Alkali and alkaline earth cations, which are present in many oxide ceramics and almost all silicate ceramics, are not found in non-oxide ceramics, except as an impurity or—in the exception—as a dopant.

Further details on non-oxide ceramics can be found in Thieme Römpp Online 2014 Version 3.45, “Nonichtoxidkeramik”. Examples of suitable non-oxide ceramics can also be found in the American patent application US 2014/0206525 A1 and the German patent applications DE 102 07 860 A1 and DE 10 2012 021 906 A1.

Glass-ceramics are polycrystalline solids with more than 30% glass phase that are produced by controlled crystallisation of glasses. The crystals are formed by heat treatment of a suitable glass, usually colourless, and cause spatial scattering of the light entering the material.

Examples of suitable glass-ceramics are
MgOₓAl₂O₃ₓnSiO₂-System (MAS system),
ZnOₓAl₂O₃xnSiO₂ (ZAS system),
LiOₓAl₂O₃xnSiO₂ (LAS system) und
KMg₃[(F,OH)₂AlSi₃O₁₀] (phlogopite).

Further details on glass-ceramics can be found in Thieme Römpp Online 2014 Version 3.45, “Glasskeramik”, in WO 2010/081561 A1, “Optically permeable glass and glass-ceramic foams, process for their preparation and their use” as well as in the article by A. M. Marques and A. M. Bernardin, “Ceramic Foams made from Plain Glass Cullets”, Qualicer 2008, pages 89 to 93.

Particularly preferably calcium silicates are used.

Calcium silicates are common and known products available on the market and can be produced by a hydrothermal process from the finely milled raw materials lime and sand in a water suspension with a low solids content and additives. The mineralogical transformation into the main phases tobermorite 5CaO×6SiO₂×5.5 H₂O (approx. 10% water, stable up to 650° C.) and xonolite 6CaO×6SiO₂×H₂O (approx. 3% water, stable up to 850° C.) takes place in autoclaves. The anhydrous phase wollastonite 3CaO×3SiO₂ as an additive increases the temperature resistance. The dewatering reactions determine the degree of shrinkage and thus the application limits of the material.

Heavy metal-free inorganic particles of grinding aids can be added to the material to be ground F. These grinding aids can accelerate the grinding process and the mechanochemical carbonisation of the POPs and PAHs. Examples of suitable grinding aids include quartz, glass, aluminium nitride, silicon nitride, silicon carbide, silicon carbide nitride, aluminium oxide and boron nitride. With the aid of these grinding aids also triboplasma is produced, which further accelerates the mechanochemical reactions.

The quantities of activated carbon particles A used in the topical pharmaceuticals, medical products and cosmetics according to the invention can vary very widely and depend mainly on their intended use. Preferably, the amounts used are 0.1% by weight to 50% by weight, preferably 0.5% by weight to 40% by weight, particularly preferably 1.0% by weight to 30% by weight and especially 2% by weight to 20% by weight, in each case based on the total amount of a given topical pharmaceutical, medical product and cosmetic according to the invention. The high dosages can be used for special applications. The topical pharmaceuticals, medical products and cosmetics in question then take the form of viscous pastes or slurries with a content of activated carbon particles A of up to 50% by weight.

Particularly advantageous topical pharmaceuticals according to the invention contain particles B of an average particle size d₅₀ equal to 10 nm to <1000 μm, preferably 20 nm to 1000 nm, preferably 30 nm to 800 nm and in particular 40 nm to 600 nm. The particles B contain and/or consist of at least one, in particular one, type of nanocrystalline and/or microcrystalline natural waxes and/or semi-synthetic waxes based on natural waxes and/or biodegradable microplastics.

The term “wax” is a phenomenological term for a number of naturally or artificially or synthetically obtained substances which generally have the following properties: kneadable at 20° C., solid to brittle-hard, coarse to fine crystalline, translucent to opaque, not glassy, melting above 40° C. without decomposition, but already relatively low viscosity slightly above the melting point and generally or advantageously not stringy, have a strongly temperature-dependent consistency and solubility and can be polished under slight pressure. If more than one of the properties listed above is not fulfilled, this substance is not a wax according to the DGF (German Society for Fat Science) (see DGF Einheitsmethode M-I 1 (75)). Waxes differ from similar synthetic or natural products such as resins, plastic masses, metal soaps, etc. mainly in that they generally change to the molten, low-viscosity state between about 50° C. and 90° C., in exceptional cases also up to about 200° C., and are practically free of ash-forming compounds. Waxes form pastes or gels and usually burn with a sooty flame. Further details on the term “waxes” can be found in Römpp Chemielexikon, 10th edition, volume 6, 19 199, Georg Thieme Verlag Stuttgart/New York, page 4.9.2006, keyword: “Wachse”.

According to the invention, it is preferred if a wax B is used which contains polar functional groups, in particular groups which contain heteroatoms from the group of oxygen, nitrogen and/or sulphur, preferably oxygen, particularly preferably hydroxyl groups, polyether groups, in particular polyethylene oxide groups, and/or carboxyl groups.

Examples of suitable natural waxes are vegetable waxes such as xanthan gum, candelilla wax, carnauba wax, Japan wax, esparto grass wax, cork wax, guaruma wax, rice germ oil wax, sugar cane wax, ouricury wax, montan wax, absolute flower waxes, Cuban palm wax, cotton wax, flax wax, peat wax, rose wax, jasmine wax, peetha wax, myrtle wax and wax fig wax.

Examples of suitable natural waxes are animal waxes such as spermaceti, wool wax, shellac, China wax, beeswax and brush gland wax.

Examples of suitable natural waxes are mineral waxes such as ceresin and ozokerite.

Examples of suitable modified natural waxes are hard waxes, montan ester waxes, sasol waxes, hydrogenated jojoba waxes, lanolin alcohols (Eucerit) and modified beeswaxes (Cerabellina).

Beeswaxes, cerabellina and absolute flower waxes are particularly preferred.

Preferably, the wax particles B are produced from natural and semi-synthetic waxes using the GAS process (Gas Antisolvent Recrystallisation), the PCA process (Precipitation with a Compressed Fluid Antisolvent), the PGSS process (Particles from Gas Saturated Solutions) or the RESS process (Rapid Expansion of Supercritical Solutions) (see, for example, the American patent applications US 2012/0258150 A1 and US 2013/0259913 A1).

Instead of or in addition to the wax particles B, the topical pharmaceuticals, medical products and cosmetics according to the invention may contain biodegradable and non-biodegradable microplastics or microbeads, which is, however, less preferred according to the invention. If non-biodegradable microplastics are used, their quantities should be kept as low as possible.

Suitable biodegradable microbeads B are known, for example, from American patent application 2014/0026916 A1. They have an average particle size d₅₀<400 μm and consist of polyhydroxyalkanoates. Polyhydroxyalkanoates (PHA) or polyhydroxy fatty acids (PHF) are naturally occurring water-insoluble and linear polyesters that are formed by many bacteria as carbon and energy reserves. In nature, they are produced by fermentation of sugar or fats. These biopolymers are biodegradable and are used in the preparation of bioplastics. They have the advantage that they sink to the bottom in water and biodegrade. Other suitable biodegradable microbeads B are known, for example, from the American patent application US 2010/0278882 A1. They consist of cross-linked proteins, such as silk proteins, and have an average particle size d₅₀ of 0.1 μm to 100 μm. Other suitable biodegradable microbeads B are also known from the European patent application EP 0 563 876 A2. They are produced by cross-linking natural proteins such as gelatine, albumin and casein with glyceraldehyde as a cross-linking agent.

The average particle size d₅₀ of the wax particles B and/or the biodegradable microbeads B can vary widely and can thus be perfectly adapted to the requirements of the individual case. Preferably, the average particle size d₅₀ is between 1 μm and <1000 μm, preferably 10 μm to 700 μm, particularly preferably 50 μm to 500 μm and especially 100 μm to 300 μm.

The wax particles B and/or the biodegradable microbeads B can be protected from aggregation or agglomeration by protective colloids. Protective colloids are usually water-soluble, preferably biodegradable, polymers such as partially saponified polyvinyl acetate, polyvinyl alcohol, polyvinyl pyrrolidone, cellulose ethers (tylose) such as for example methyl cellulose, hydroxyethyl cellulose, hydroxypropyl methyl cellulose, starch, proteins, alginates, pectins and gelatins.

The content of wax particles and/or biodegradable microbeads B in the topical pharmaceuticals, medical products and cosmetics according to the invention can vary widely and can thus be excellently adapted to the respective intended use. Preferably, a given topical preparation according to the invention contains, based on its total amount, 0.1 wt. % to 50 wt. %, 0.5 to 45 wt. % and in particular 1 wt. % to 40 wt. % of wax particles B.

Further advantageous topical pharmaceuticals, medical products and cosmetics according to the invention contain particles C of an average particle size d₅₀ of 10 nm to <1000 nm, preferably 300 nm to 900 nm, preferably 650±200 nm and in particular 650±100 nm. They contain at least one type and/or consist of at least one type of nanocrystalline, nanofibrillar, microcrystalline and/or microfibrillar polysaccharides. In particular, cellulose nanofibres (CNF), microfibrillar celluloses (MFC), microcrystalline celluloses (MCC), nanofibrillar polysaccharides (NFP), nanocrystalline celluloses (CNC), bacterial nanocelluloses (BNC) and waste from paper mashes from paper preparation and fibres and particles from used textiles are used.

The content of particles C in the topical pharmaceuticals, medical products and cosmetics according to the invention can vary widely and can thus be perfectly adapted to the respective intended use. Preferably, a given topical preparation according to the invention contains, based on its total amount, 0.01% to 10% by weight, preferably 0.02% to 9% by weight and in particular 0.03% to 8% by weight.

In addition, the topical pharmaceuticals according to the invention may contain at least one ingredient D commonly used for topical medicaments, selected from the group consisting of water, alcohols, layered silicates, milled clays, bolus americus clays, healing clays, pharmacologically approved salts, nutshells, willow bark, lapach bark, silica, pumice stones, geopolymers, amber, gemstones, trace elements and neutral inorganic salts, beta-glucans, glutathione, albumin, xylan, phytates, complexing agents, antioxidants, radical scavengers, menthol, caffeine, teein, papain, esculin semihydrate, carotenoids, lecithins, abscisic acid, cyanidin, guanidin, urea, thiourea, fillers, solvents, humectants, solubilisers, non-ionic wetting agents, buffers, thickeners, binders, coating agents, disintegration accelerators, disintegrants, anti-friction agents, lubricants, mould release agents, flow regulators, antioxidants, preservatives, sweeteners, flavour corrigents, absorption accelerators, alkalising agents, acidifying agents, neutralising agents, foam inhibitors and defoamers, dyes, colour pigments, oxidising agents, reducing agents, stabilisers, propellants, opacifiers, pearlescent agents, denaturants, emollients, minerals, vitamins, micelles, superabsorbents, refatting substances, oils, fragrances, flavourings, perfumes, moisturisers, moisturising agents, active ingredients for skin care, deodorising agents, film formers, gelling agents, tannins, tanning agents, tanning extracts, bittering agents, plant extracts, honeys, colostrum, cytostatic agents, antibodies, immunoglobulins, essential and non-essential amino acids and fats, hyaluronic acid, aloe vera gel, alkylene glycols, polyalkylene ether glycols, proteins, peptides, carbohydrates, lipids, nucleic acids, nucleic acid fragments, antiviral compounds, disinfectants, antibiotics, cell differentiation reagents, narcotics, anaesthetics, anaesthetics, contrast agents, enzymes, cytokines, antihistamines, immunomodulators, haemostatic reagents, hormones, angiogenic and antiangiogenic agents, neurotransmitters, therapeutic oligonucleotides, virus particles, growth factors, retinoids, cell adhesion factors, extracellular matrix glycoproteins, osteogenic factors, antibodies, antigens, steroids, painkillers, potassium sulphate, royal jelly, propolis, encapsulated fragrances, nutrients hexahydroxycyclohexane hexaphosphoric acid ester salt nanoparticles, in particular salts of phytic acid, and surfactants, in effective amounts.

Examples of suitable ingredients D are in particular layered silicates, which are preferably present as nanoparticles and/or microparticles of an average particle size d₅₀ of 1 nm to <1000 μm, preferably 10 nm to 900 μm, preferably 300 nm to 1000 nm, particularly preferably 650±200 nm, most preferably 650±150 nm and in particular 650±100 nm.

The elemental composition and structure of the layered silicate micro- and/or nanoparticles can also vary widely. For example, silicates are known to be categorised into the following structures: island silicates, group silicates, ring silicates, chain and ribbon silicates, transition structures between chain and layer silicates, layer silicates, framework silicates.

Layered silicates are silicates whose silicate ions consist of layers of corner-linked SiO₄-tetrahedra. These layers and/or double layers are not linked to each other further. The technically important clay minerals that are common in sedimentary rock are also layered silicates. The layered structure of these minerals determines the shape and properties of the crystals. They are usually tabular to foliated with good to perfect cleavability parallel to the layers. The countability of the rings that make up the silicate layers often determines the symmetry and shape of the crystals. Water molecules, large cations and/or lipids can be deposited between the layers. Examples of suitable heavy metal-free layered silicates are shown in Table 1 below. The list is exemplary and not exhaustive.

TABLE 1
Sum formulae of suitable heavy metal-free layered silicates a)

| type | molecular formula |
|---|---|
| Martinite | (Na,Ca)₁₁Ca₄(Si,S,B)₁₄B₂O₄₀F₂•4(H₂O) |
| Apophyllite-(NaF) | NaCa₄Si₈O₂₀F•8H₂O |
| Apophyllite-(KF) | (K,Na)Ca₄Si₈O₂₀(F,OH)•8H₂O |
| Apophyllite-(KOH) | KCa₄Si₈O₂₀(OH,F)•8H₂O |
| Natrosilite | Na₂Si₂O₅ |
| Kanemite | NaSi₂O₅•3H₂O |
| Revdite | Na₁₆Si₁₆O₂₇(OH)₂₆•28H₂O |
| Latiumite | (Ca,K)₄(Si,Al)₅O₁₁(SO₄,CO₃) |
| Tuscanite | K(Ca,Na)₆(Si,Al)₁₀O₂₂(SO₄,CO₃,(OH)₂)•H₂O |
| Carletonite | KNa₄Ca₄Si₈O₁₈(CO₃)₄(OH,F)•H₂O |
| Pyrophyllite | Al₂Si₄O₁₀(OH)₂ |
| Talc | Mg₃Si₄O₁₀(OH)₂ |
| Paragonite | NaAl₂(Si₃Al)O₁₀(OH)₂ |
| Boromuscovite | KAl₂(Si₃B)O₁₀(OH,F)₂ |
| Muscoviet | KAl₂(Si₃Al)O₁₀(OH,F)₂ |
| Tobelite | (NH₄,K)Al₂(Si₃Al)O₁₀(OH)₂ |
| Nanpingite | CsAl₂(Si,Al)₄O₁₀(OH,F)₂ |
| Polylithionite | KLi₂AlSi₄O₁₀(F,OH)₂ |
| Tainiolite | KLiMg₂Si₄O₁₀F₂ |
| Shirokshiniet | KNaMg₂[F₂\|Si₄O₁₀] |
| Trilithionite | KLi_{1.5}Al_{1.5}[F₂\|AlSi₃O₁₀] |
| Aspidolithe-1M | NaMg₃(AlSi₃)O₁₀(OH)₂ |
| Fluorophlogopite | KMg₃(AlSi₃)O₁₀F₂ |
| Phlogopite | KMg₃(Si₃Al)O₁₀(F,OH)₂ |
| Ephesite | NaLiAl₂(Al₂Si₂)O₁₀(OH)₂ |
| Preiswerkite | NaMg₂Al₃Si₂O₁₀(OH)₂ |
| Eastonite | KMg₂Al[(OH)₂\|Al₂Si₂O₁₀] |
| Margarite | CaAl₂(Al₂Si₂)O₁₀(OH)₂ |
| Clintonite | Ca(Mg,Al)₃(Al₃Si)O₁₀(OH)₂ |
| Illite | (K,H₃O)Al₂(Si₃Al)O₁₀(H₂O,OH)₂ |
| Agrellite | NaCa₂Si₄O₁₀F |
| Glagolevite | NaMg₆[(OH,O)₈\|AlSi₃O₁₀]•H₂O |
| Lunijianlaite | Li_{0.7}Al_{6.2}(Si₇AlO₂₀)(OH,O)₁₀ |
| Saliotit | Na_{0.5}Li_{0.5}Al₃[(OH)₅\|AlSi₃O₁₀] |
| Kulkeit | Na_{0.35}Mg₈Al(AlSi₇)O₂₀(OH)₁₀ |
| Aliettite | Ca_{0.2}Mg₆(Si,Al)₈O₂₀(OH)₄•4H₂O |
| Rectorite | (Na,Ca)Al₄(Si,Al)₈O₂₀(OH)₄•2H₂O |
| Tarasovite | (Na,K,H₃O,Ca)₂Al₄[(OH)₂\|(Si,Al)₄O₁₀]₂•H₂O |
| Tosudite | Na_{0.5}(Al,Mg)₆(Si,Al)₈O₁₈(OH)₁₂•5H₂O |
| Montmorillonite | (Na,Ca)_{0.3}(Al,Mg)₂Si₄O₁₀(OH)₂•nH₂O |
| Beidellite | (Na,Ca_{0.5})_{0.3}Al₂(Si,Al)₄O₁₀(OH)₂•4H₂O |
| Swinefordite | (Ca,Na)_{0.3}(Al,Li,Mg)₂(Si,Al)₄O₁₀(OH,F)₂•2H₂O |
| Hectorite | Na_{0.3}(Mg,Li)₃Si₄O₁₀(F,OH)₂ |
| Spadaite | MgSiO₂(OH)₂•H₂O |
| Stevensite | (Ca\|₂)_{0.3}Mg₃Si₄O₁₀(OH)₂ |
| Donbassite | Al_{2.3}[(OH)₈\|AlSi₃O₁₀] |
| Sudoite | Mg₂Al₃(Si₃Al)O₁₀(OH)₈ |
| Cookeite | LiAl₄(Si₃Al)O₁₀(OH)₈ |
| Borocookeite | Li_{1-1.5}Al_{4-3.5}[(OH,F)₈\|(B,Al)Si₃O₁₀] |
| Manandonite | Li₂Al₄[(Si₂AlB)O₁₀](OH)₈ |
| Kaolinite | Al₂Si₂O₅(OH)₄ |
| Dickite | Al₂Si₂O₅(OH)₄ |
| Halloysite-7Å | Al₂Si₂O₅(OH)₄ |
| Allophan | Al₂O₃•(SiO₂)_{1.3-2}•(H₂O)_{2.5-3} |
| Imogolite | Al₂SiO₃(OH)₄ |
| Chrysotile | Mg₃Si₂O₅(OH)₄ |
| Clinochrysotile | Mg₃Si₂O₅(OH)₄ |
| Orthochrysotile | Mg₃Si₂O₅(OH)₄ |
| Parachrysotile | Mg₃Si₂O₅(OH)₄ |
| Lizardite | Mg₃Si₂O₅(OH)₄ |
| Amesite | Mg₂Al(SiAl)O₅(OH)₄ |
| Palygorskite | (Mg,Al)₂Si₄O₁₀(OH)•4H₂O |
| Loughlinite | Na₂Mg₃Si₆O₁₆•8H₂O |
| Kalifersite | (K,Na)₅Fe₇³⁺[(OH)₃\|Si₁₀O₂₅]₂•12H₂O |
| Minehillite | (K,Na)₂₋₃Ca₂₈(Zn₄Al₄Si₄₀)O₁₁₂(OH)₁₆ |
| Fedorite | (Na,K)₂₋₃(Ca,Na)₇[Si₄O₈(F,Cl,OH)₂\|(Si₄O₁₀)₃]•3.5H₂O |
| Reyerit | (Na,K)₄Ca₁₄Si₂₂Al₂O₅₈(OH)₈•6H₂O |
| Gyrolite | NaCa₁₆Si₂₃AlO₆₀(OH)₈•14H₂O |
| Zeophyllite | Ca₄Si₃O₈(OH,F)₄•2H₂O |
| Mountainite | KNa₂Ca₂[Si₈O₁₉(OH)]•6H₂O |
| Rhodesite | KHCa₂Si₈O₁₉•5H₂O |
| Delhayelithe | K₇Na₃Ca₅Al₂Si₁₄O₃₈F₄Cl₂ |
| Hydrodelhayelithe | KCa₂AlSi₇O₁₇(OH)₂•6H₂O |
| Tienshanite | (Na,K)₉₋₁₀(Ca,Y)₂Ba₆(Mn²⁺,Fe²⁺,Ti⁴⁺,Zn)₆(Ti,Nb) |
| | [(O,F,OH)₁₁\|B₂O₄\|Si₆O₁₅]₆ |
| Silhydrite | Si₃O₆•H₂O |
| Magadiite | Na₂Si₁₄O₂₉•11H₂O |
| Strätlingite | Ca₂Al[(OH)₆AlSiO₂(OH)₄]•2.5H₂O |
| Vertumnite | Ca₄Al₄Si₄O₆(OH)₂₄•3H₂O |

a) see Mineral Atlas, Mineral Class VIII/H - Layered silicates, Strunz 8 Systematic

Heavy metal-free bentonite from the group of montmorillonites ((Na,Ca)_{0.3}(Al,Mg)₂Si₄O₁₀(OH)₂-nH₂O) is particularly favoured. Bentonite is a mixture of different clay minerals and contains montmorillonite as its most important component. Sodium bentonite, for example, takes up water, it can absorb a plurality of its own dry weight. Calcium bentonite can also take up fats and/or oils.

The layered silicate micro- and/or nanoparticles described above are functionalized, non-functionalized, aggregated, non-aggregated, agglomerated, non-agglomerated, supported and/or non-supported. For example, they can be functionalised, agglomerated and supported. However, they can also be non-functionalised and aggregated.

Examples of other suitable, preferably halogen-free, ingredients D are lactose, cellulose, starches, sucrose (tablets); paraffin (ointments); hard fat (suppositories); polyethylene glycols (macrogols, PEG); polyethylene oxides (PEO) (tablets, ointments, creams); solvents and/or moisturisers: VC water, ethanol, isopropanol (granulation, film spraying); emulsifiers: Cetylstearyl alcohol, gylcerol monostearate, lecithin, fatty acid esters of sorbitan, of polyoxyethylene sorbitan (polysorbates), of polyoxyethylene, of polyoxyethylene fatty alcohol ether (emulsions, creams), methyl glucose, sesquistearates, stearic acid and its derivatives, acrylate-based emulsifiers such as acrylates/C10-330 alkyl acrylate crosspolymer; solubilisers and/or wetting agents: polyethylene glycols (PEG, macrogols), polyethylene oxides (PEO, PolyOx), polysorbates (solutions, suspensions); buffers: dipotassium hydrogen phosphate, sodium dihydrogen phosphate, sodium hydrogen carbonate, calcium hydrogen phosphate, trometamol (solutions, creams); thickening and binding agents: starches, guaran, xanthan gum, alginate, carrageenan, pectin, tragacanth, polyacrylic acids, polyvinylpyrrolidone (granules, tablets); highly dispersed silicon dioxide, substituted cellulose ethers (methyl cellulose, ethyl cellulose, hydroxypropyl cellulose, hydroxypropyl methyl cellulose, carboxymethyl cellulose) (tablets, gels, viscous solutions), butyrospermum parkii butter, magnesium sulphate; coating agent: sucrose (sugar coating); gelatine (capsules); gelatine polysuccinate (“soft capsules”), polyacrylates, ethylcellulose, methylcellulose (film coating: film-coated capsules, film-coated tablets, pellets); disintegration accelerators and/or disintegrants: starches (tablets, tabs); croscarmellose (tablets, capsules, granules); sodium hydrogen carbonate in combination with citric acid (effervescent tablets); lubricants and/or mould release agents: polyethylene glycols (PEG, macrogols), polyethylene oxides (PEO), talc, magnesium stearate (tableting); flow regulators: highly dispersed silicon dioxide (powder, granules); antioxidants: butylated hydroxytoluene, all-rac-α-tocopherol; preservatives: PHB esters, benzalkonium chloride, benzyl alcohol, thiomersal; sweeteners, taste correctors: sucrose, sorbitol, sweeteners such as saccharin sodium and cyclamate; flavourings; absorption accelerators: dimethyl sulphoxide (in topical medicines).

Other aids are among others

### Alkalising Agents/Acids/Neutralising Agents

Acetic acid, 2-amino-2-methylpropanol, ammonia, ammonium hydrogen carbonate, ammonium carbonate, citric acid, disodium hydrogen phosphate, disodium dihydrogen pyrophosphate, 2-aminoethanol, hydrogen chloride, phosphoric acid, potassium citrate, potassium dihydrogen phosphate, sodium acetate, sodium hydrogen carbonate, sodium citrate, sodium hydroxide, sodium metaphosphate, sodium dihydrogen phosphate, trisodium trimetaphosphate, potassium pyrophosphate, sodium pyrophosphate, tris(2-hydroxyethyl)amine, trihydroxytriethylamine;

### Antioxidants

Ascorbic acid, palmitic acid ascorbyl ester, L-ascorbyl palmitate, butylhydroxytoluene, 2,6-di-t-butyl-p-cresol, carotenoids, lycopene, alpha-tocopherol, vitamin E, alpha-tocopheryl acetate (vitamin E acetate), Q10, coenzyme Q10; ubiquinone-10, rutin;

### Binding Agents

Alkyd resins, emulsions, epoxy resins, burnt lime, cement, clay and potassium water glas, gum arabic, resins, honey, casein, plastic dispersions, vegetable oils (linseed oil, avocado oil, wheat germ oil, almond oil, lavender oil), polyurethanes, rosemary, silicone resin, wax, cellulose glue (paste);

### Dyes/Colour Pigments,

Sodium 4-[(4,5-dihydro-3-methyl-5-oxo-1-phenyl-1H-pyrazol-4-yl)azo]-3-hydroxnaphthalene-1-sulfonate, dihydroxyaluminium stearate, anthocyanins (cyanidin, peonidin, malvidin, delphinidin, petunidin, pelargonidin), beetroot; beetroot; sugar beet, calcium stearate, paprika extract; capsanthin, capsorubin, caramel, caramelised sugar, 1,4-bis(p-tolylamino)anthraquinone, sodium 1-amino-4-(cyclohexylamino)-9,10-dihydro-9,10-dioxoanthracene-2-sulphonate, indigo; 2-(1,3-Dihydro-3-oxo-2H-indazol-2-yliden)-1,2-dihydro-3H-indol-3-on, Dinatrium[29H,31H-phthalocyanindisulfonato(4-)-N29,N30,N31,N32]cuprat(2-), guanine; 2-amino-6-hydroxypurine; 2-amino-1,7-dihydro-6H-purin-6-one, curcumin, curcumin; 1,7-bis(4-hydroxy-3-methoxyphenyl)hepta-1,6-diene-3,5-dione, carmine; carminic acid, chlorophyll; chlorophyll a/b; trisodium-(2S-trans)-[18-carboxy-20-(carboxymethyl)-13-ethyl-2,3-dihydro-3,7,12,17-tetramethyl-8-vinyl-21H,23H-porphin-2-propionato(5-) N21,N22,N23,N24]cuprate(3-), aluminium, aluminium oxide hydrate; aluminium hydroxide, hydrous aluminium silicate, calcium carbonate, calcium sulphate, calcium sulphate, magnesium carbonate, lactoflavin, riboflavin, magnesium distearate, riboflavin, lactoflavin;

### Humectant

Cyclomethicone; consists of octamethylcyclotetrasiloxane (D4), decamethylcyclopentasiloxane (D5) and dodecamethylcyclohexasiloxane (D6), decamethylcyclopentasiloxane, 1,2,3-propanetriol, ethylene glycol, lactic acid, y6, polyethylene glycol (average 6 units —CH2-CH2-O—), sodium lactate, sodium hyaluronate, sorbitol, sorbitol, glucitol, panthenol (provitamin B5), Camellia Euryoides Leaf Extract;

### Resins

Polymethacrylic acid, polyvinyl acetate, toluenesulphonamide formaldehyde resin;

### Preservatives

4-Hydroxybenzoic acid, ammonium benzoate, ammonium bisulphite, ammonium propionate, ammonium sulphite, benzethonium chloride, benzoic acid, benzyl alcohol; phenylmethanol, benzoic acid butyl ester, butylparaben; 4-hydroxybenzoic acid butyl ester, calcium benzoate, calcium paraben, 4-hydroxybenzoic acid, calcium salt, calcium propionate, calcium salicylate, calcium sorbate, cetrimonium chloride, dehydroacetic acid, 3-acetyl-6-methyl-2H-pyran-2,4(3H)-dione, diazolidinyl urea, DMDM hydantoin, 1,3-bis(hydroxymethyl)-5,5-dimethylimidazolidine-2,4-dione, benzoic acid ethyl ester, ethyl paraben; 4-Hydroxybenzoic acid ethyl ester, formaldehyde, formic acid, glutaral, glutardialdehyde, hexamidine, imidazolidinyl urea, 3-iodo-2-propynylbutylcarbamate, benzoic acid isobutyl ester, benzoic acid isopropyl ester, isopropyl cresols, 4-isopropyl-m-cresol, magnesium benzoate, magnesium propionate, magnesium salicylate, MEA benzoates, benzoic acid, (mono-)ethanolamine salt, methyl benzoate, methyl isothiazolinone, 2-methyl-2H-isothiazol-3-one, methyl paraben; 4-hydroxybenzoic acid methyl ester, o-phenylphenol, 2-phenoxyethanol, phenoxyisopropanol, 1-phenoxypropan-2-ol, benzoic acid phenyl ester, piroctone olamine, potassium benzoate, butylparaben (potassium salt); 4-hydroxybenzoic acid butyl ester (potassium salt), ethyl paraben (potassium salt); 4-hydroxybenzoic acid ethyl ester (potassium salt), dipotassium disulphite, methyl paraben (potassium salt); 4-hydroxybenzoic acid methyl ester (potassium salt), 4-hydroxybenzoic acid, potassium salt, potassium propionate, propyl paraben (potassium salt); 4-hydroxybenzoic acid propyl ester (potassium salt), potassium salicylate, potassium sorbate, potassium sulphite, propionic acid, benzoic acid propyl ester, propyl paraben; 4-hydroxybenzoic acid propyl ester, sodium benzoate, sodium hydrogen sulphite, butyl paraben (sodium salt); 4-hydroxybenzoic acid butyl ester (sodium salt), ethyl paraben (sodium salt); 4-hydroxybenzoic acid ethyl ester (sodium salt), sodium formate, disodium disulfite, sodium metabisulfite, methyl paraben (sodium salt); 4-hydroxybenzoic acid methyl ester (sodium salt), 4-hydroxybenzoic acid, sodium salt, sodium propionate, propyl paraben (sodium salt); 4-hydroxybenzoic acid propyl ester (sodium salt), sodium salicylate, sodium sorbate, sodium sulphite, sorbic acid, 2,4-hexadienoic acid, stearalkonium chloride, ammonium peroxodisulphate, ammonium persulphate, hydrogen peroxide, potassium persulphate, potassium peroxodisulphate, sodium persulphate, sodium peroxodisulphate;

### Reducing Agent,

Ammonium hydrogen sulphite, ammonium thioglycolate, ammonium mercaptoacetate, disodium disulphite, sodium metabisulphite, thioglycolic acid, mercaptoacetic acid, thiomlichic acid, 2-mercaptopropionic acid;

### Stabilisers

Acrylates/C10-30 alkyl acrylate crosspolymer, polymer of alkyl acrylates (C10-C30), acrylic acid, methacrylic acid and/or other alkyl acrylates; cross-linked with propenyl (allyl) modified sugars, palmitic acid ascorbyl ester, L-ascorbyl palmitate, bentonite (clay mineral), BHT, butylhydroxytoluene, 2,6-di-t-butyl-p-cresol, carbomer, polyacrylic acid, cetearyl alcohol, mixture of 1-hexadecanol (cetyl alcohol) and 1-octadecanol (stearyl alcohol), 2-hydroxyethylcellulose cetyl ether, cocamide MEA, N-(hydroxyethyl)-coconut fatty acid amide, ethylenediaminetetraacetic acid, disodium salt, ethylenediaminetetraacetic acid, Etidronic acid, 1-hydroxyethylidene bis-phosphonic acid, hydroxyethyl cellulose, cellulose (microcrystalline), pentasodium (carboxylatomethyl)iminobis(ethylene nitrilo)tetraacetate, potassium alginate (potassium salt of alginic acid), polyvinylpyrrolidone, sodium carbomer, polyacrylic acid (sodium salts), sodium citrate, sodium gluconate, ethylenediaminetetraacetic acid, tetrasodium salt, alpha-tocopherol, vitamin E, ethylenediaminetetraacetic acid, trisodium salt, polysaccharides;

### Propellants for Sprays

Butane, carbon dioxide, dimethyl ether, isobutane, 2-methylpropane, nitrogen, propane.

Opacifiers/pearl lustre agents, for example: calcium sulphate; cellulose; palmitic acid glycol ester; 2-hydroxyethyl palmitate; mica (a silicate mineral); peg-2 stearate, 2-(2-hydroxyethoxy)ethyl stearate; esters of stearic acid and diethylene glycol (1:1); propylene glycol distearate, propylene glycol distearate; ester of stearic acid and propylene glycol (2:1); propylene glycol stearateE SE, propylene glycol stearate; ester of stearic acid and propylene glycol (1:1), contains sodium or potassium stearate; denaturant; plasticiser, butyl stearate; camphor; cetearyl isononanoate, dibutyl sebacate, dicaprylyl ether, dioctyl ether; diisodecyl adipate; isopropyl citrate; isopropyl myristate; isopropyl palmitate; octyldodecanol; 2-octyldodecan-1-ol; sorbitol, sorbitol, glucitol; toluenesulphonamide formaldehyde resin; minerals; sand; vitamins: Vitamin A, vitamin B1, vitamin B2, vitamin B3, vitamin B4, vitamin B5, vitamin B6, vitamin B7, vitamin B8, vitamin B9, vitamin B12, vitamin C, vitamin D, vitamin E, vitamin K; salt: table salt, Himalayan salt, Hawaiian salt, Dead Sea salt; micelles.

Moisturising substances: moisturising substances, also known as regreasing agents and/or refatting agents, are substances that are usually added to surfactant preparations to counteract their degreasing nature. The following refatting substances are used for this purpose. The following list is exemplary and not exhaustive. The skilled person can easily name other possible refatting substances based on his general technical knowledge. For example: Squalane, 2,6,10,15,19,23-Hexamethyltetracosan; squalene, 2,6,10,15,19,23-Hexamethyl-2,6,10,14,18,22-tetracosahexaen; steareth-10, polyethylene glycol ether of stearyl alcohol (average 10 units —CH₂—CH₂—O—); stearyl alcohol; trimyristine, glyceryl trimyristate; esterification product of glycerol and myristic acid (1:3); tripalmitin, euceritol, lanolin alcohols; glyceryl tripalmitate; esterification product of glycerin and palmitic acid (1:3); wheat germ glycerides, esterification product of glycerin and fatty acids from wheat germ oil.

Oils: Oils are a collective term for various liquid fatty substances. These include among others mineral oils, silicone oils and/or oils of natural origin, such as vegetable oils and/or essential oils. These are often characterised by monounsaturated and/or polyunsaturated fatty acids. Oils of animal origin are usually referred to as fats. In addition, many oil components used in cosmetics are produced synthetically on the basis of natural raw materials. Just like the moisturising substances, oil components prevent the skin and/or hair from drying out prematurely. In the following list, the oils are listed by way of example and not exhaustively. The skilled person can easily name further possible oils based on his general technical knowledge. For example: Betula alba bark extract (birch); caprylic acid/capric acid triglyceride, esterification product of glycerine and capric acid as well as caprylic acid (1:3); cetearyl alcohol, mixture of 1-hexadecanol (cetyl alcohol) and 1-octadecanol (stearyl alcohol); cyclomethicone; consists of octamethylcyclotetrasiloxane (D4), decamethylcyclopentasiloxane (D5) and dodecamethylcyclohexasiloxane (D6); Dibutyl sebacate; dicaprylyl ether, dioctyl ether; diisodecyladipate; polydimethylsiloxane; glyceryl stearate, glyceryl monostearate; isopropyl myristate; isopropyl palmitate, cocoa butter; Macadamia integrifolia seed oil, macadamia; real macadamia nut; macadamia nut oil; Macadamia ternifolia seed oil, macadamia, trifoliate; macadamia nut oil; octyldodecanol, 2-octyldodecan-1-ol; paraffin, hard paraffin, microcrystalline wax, ozokerite, ceresin, vaseline; paraffinum liquidum; liquid paraffin, paraffin oil, mineral oil; Persea gratissima oil, avocado; avocado oil; shea butter; Simmondsia chinensis seed oil; jojoba shrub; jojoba oil; stearic acid; fragrance and/or flavouring.

### Essential Oils, Flavourings, Fragrances and Perfumes:

Essential oils are volatile, intensely smelling mixtures of substances with an oily consistency that are poorly soluble in water. They are usually obtained from plant-based raw materials by steam distillation. They can also be obtained by synthesis, extraction or pressing. Pure plant substances such as menthol, cineole, vanillin and/or thymol are also referred to as essential oils. Depending on the oil, they are used in medicine to treat numerous ailments, including muscle and joint pain, infectious diseases, colds and gastrointestinal complaints. They are also used in cosmetics, especially for perfuming.

Flavourings are products that are added to foods, for example, to give them a special smell and/or taste. A flavouring can consist of numerous flavouring substances, flavouring extracts, thermally obtained reaction flavourings, smoke flavourings and/or flavour precursors. Most flavourings are used industrially in food preparation, but some flavourings are also available in retail outlets. Flavouring substances are chemically defined substances with flavouring properties that are used in the preparation of flavourings. A distinction is made between natural, nature-identical and/or artificial flavouring substances.

Fragrances used in cosmetics are, on the one hand, organic compounds from the animal and plant kingdom. Fragrances are also compounds that can be produced synthetically. The purpose of fragrances in cosmetic products is to mask the inherent odour of a product and further to trigger pleasant sensations in the consumer. Perfumes in the narrower sense are fragrances and fragrance mixtures that are intended to alter or mask body odour.

The following list of essential oils, flavourings, fragrances and perfumes is exemplary and not exhaustive. The skilled person can easily name further possible essential oils, flavours and/or fragrances and/or perfumes on the basis of his general technical knowledge.

Essential oils, for example: camphor; Cinnamomum zeylanicum leaf oil, Ceylon cinnamon tree; cinnamon laurel tree; Citrus limon peel oil, lemon tree; lemon; eucalyptol; 3,3-trimethyl-2-oxabicyclo[2.2.2]octane, 1,8-cineole; Jasminum officinale flower extract, jasmine; Lavandula angustifolia oil, true lavender; sweet almond oil; menthol; Pinus sylvestris leaf oil, pine, common; vanillin.

Flavouring substances, for example: flavouring substance, flavouring mixture; menthol; 5-methyl-2-(1-methylethyl)cyclohexanol; vanillin.

Perfumes and/or fragrances, for example: anis alcohol; anisyl alcohol, 4-methoxybenzyl alcohol; benzyl alcohol; phenylmethanol; Cinnamomum zeylanicum leaf oil, cinnamon tree; cinnamon laurel tree; Citrus limon peel oil, lemon tree, lemon; eucalyptol; 3,3-trimethyl-2-oxabicyclo[2.2.2]octane, 1,8-cineole; Jasminum officinale flower extract, jasmine; methyl phenylbutanol, 2-methyl-4-phenylbutan-2-ol; octyldodecanol, 2-octyldodecan-1-ol; perfume.

Perfumes, fragrances and fragrance mixtures, for example: Rosa indica flower extract (tea rose); Rosa centifolia (rose water); limonene, carvene, p-mentha-1,8-diene, 1-methyl-4-prop-1-en-2-yl-cyclohexene, 1-methyl-4-isopropenyl-1-cyclohexene.

Moisturisers, for example: Aloe barbadensis leaf juice, aloe vera; gel/juice; aloe barbadensis leaf juice powder; collagen; glycerine; hyaluronic acid; lactic acid; sodium hyaluronate (sodium salt of hyaluronic acid); sodium lactate (sodium salt of lactic acid); urea; ALGAE (algae); Echinacea purpurea extract.

Active ingredients for the skin, for example: Allantoin; aloe barbadensis leaf juice, aloe vera; gel/juice; ascorbic acid; bisabolol, alpha-bisabolol, (R)-6-methyl-2-((R)-4-methylcyclohex-3-enyl)hept-5-en-2-ol; esters of benzoic acid and fatty alcohols of chain length C12 to C15; caprylic acid/capric acid triglyceride, esterification product of glycerine with capric acid and caprylic acid (1:3); Chamomilla recutita flower extract, true chamomile; decyl oleate; diethylhexyl syringylidene emalonate, [(4-hydroxy-3,5-dimethoxyphenyl)methylene]propanedioic acid, bis(2-ethylhexyl) ester; ethylhexyl cocoate, esterification product of 2-ethylhexanol and coconut fatty acids; ethylhexyl stearate, stearic acid 2-ethylhexyl ester; ethylhexylglycerol; 3-(2-ethylhexyloxy)-1,2-propanediol; Fagus silvatica (beech); hyaluronic acid; niacinamide, panthenol, dexpanthenol, provitamin B5; Persea gratissima oil, avocado; avocado oil; PPG-15 stearyl ETHER, stearyl alcohol, etherified with polypropylene glycol (average 15 units propylene glycol); propylheptyl caprylate, esterification product of 2-propylheptanol with caprylic acid; Simmondsia chinensis ceed oil, jojoba shrub; jojoba oil; sodium hyaluronate; stearyl dimethicone, poly(dimethyl-methylstearyl)siloxane; sucrose laurates, esterification product of lauric acid with sucrose (cane sugar); tocopherol, alpha-tocopherol, vitamin E; coenzyme Q10; ubiquinone-10; Helianthus annuus hybrid oil; Echium plantagineum seed oil; Cardiospermum halicacabum flower/leaf/vine extract; rutin; arginine.

Deodorising active ingredients, for example: farnesol, 3,7,11-trimethyl-2,6,10-dodecatrien-1-ol; perfume; fragrances and fragrance mixtures; triclosan, 5-chloro-2-(2,4-dichlorophenoxy)phenol; triethyl citrate; citric acid triethyl ester; zinc lactate (zinc salt of lactic acid); zinc ricinoleate.

Solvents; for example: 1,2-hexanediol; 1,2-dihydroxyhexane; acetone, propanone; alcohol, ethanol; denatured alcohol, denatured alcohol; water; butyl acetate, acetic acid butyl ester; 1,3-butanediol, butylene glycol; cyclopentasiloxanes; decamethylcyclopentasiloxane; ethyl acetate, ethyl acetate; glycerine, glycerol, 1,2,3-propanetriol; glycol, ethylene glycol; 2-propanol; 2-methyl-1,3-propanediol; 1,2-propanediol, propylene glycol; Echium plantagineum seed oil.

Film formers; for example: Acrylates copolymer, polymer of methacrylic acid, ethyl acrylate and methyl methacrylate; acrylates/hydroxyesters acrylates copolymer, copolymer of acrylic acid/methacrylic acid and their esters as well as hydroxyacrylic acid esters; acrylates/steareth-20 methacrylates crosspolymer, copolymer of steareth-20 methacrylate and acrylic acid/methacrylic acid and their esters, crosslinked with pentaerythritol allyl ether. Steareth-20-methacrylate is an ester of methacrylic acid with polyethylene glycol (aver. 20 units —CH₂—CH₂—O—); dibutyl sebacate, sebacic acid dibutyl ester; nitrocellulose, cellulose nitrate, guncotton; polyvinylpyrrolidone (PVP); sodium carbomer, polyacrylic acid (sodium salts); toluenesulphonamide formaldehyde resin; phthalic anhydride/trimellitic anhydride/glycols copolymer.

Gelling agents are, for example, agar, agar-agar; ALGIN, sodium alginate (sodium salt of alginic acid); alginic acid; carbomer, polyacrylic acid; carrageenan, irish moss; carrageen; hectorite (clay mineral); hydroxyethyl cellulose; hydroxypropyl cellulose; polyvinyl alcohol; polyvinyl pyrrolidone (PVP); sodium carbomer, polyacrylic acid (sodium salts); stearalkonium hectorite.

Tannin, tanning agents and/or tanning extracts: A distinction is made between tannin, tanning agents and/or tanning extracts. Tannins are chemical substances that are used to tan animal skins. Treatment with tannins transforms the animal skins into leather, which prevents rotting, among other things. There are also naturally occurring, i.e. natural, and artificially produced, i.e. synthetic tannins. Tanning agents are parts of plants and/or mixtures of substances that contain one and/or more tannins. The terms tannin and/or tanning agent are often used as synonyms. A tanning extract is an extract of a tanning agent. After extraction, the tannin is present in a higher concentration, without any interfering components of the starting material. Vegetable tannins, also known as tannins, are also used in medicine. Tannins are also known as flavour components of wine and tea.

Examples of suitable tannins are gallotannins; pyrocatechins; catechins; mineral tannins; aldehydes; aliphatic paraffin sulphochlorides; synthetic tannins (syntans) based on phenol derivatives; polymer tannins (resin tannins).

Bitter substances refer to all chemical compounds that have a bitter flavour. They are not a chemically uniform group, but are only characterised by their bitter taste. The following bitter substances are only examples and not exhaustive. The skilled person can easily name other possible bitter substances on the basis of his general technical knowledge. Examples of suitable bitter substances are lactucopicrin (found in chicory and all lettuces); cynarin (found in artichokes); glucosinolates (found in beet oil); lactucin (found in iceberg lettuce); premarrubiin and marrubiin; naringin (found in grapefruits and pomelos).

Colour pigments; for example dyes, coloured pigments, white pigments, fluorescent pigments and phosphorescent pigments (phosphorus) can be added to the topical pharmaceuticals, medical products and cosmetics according to the invention in a wide variety of ways. Preferred are chalk, talc, montmorillonite, clay minerals, papers, paper-like materials and/or titanium dioxide, including black titanium dioxide.

Plant extracts are extracts from plants that are used in cosmetics. The following list of plant extracts is exemplary and not exhaustive. The skilled person can easily name further possible plant extracts.

Examples of suitable plant extracts: African copal bark CO₂-se extract; angelica root CO₂-se extract; antimicrobial blend; arnica flowers CO₂-to extract, 4% sesquiterpene lactones; beard lichen CO₂-to extract, water dispersible, 4% usnic acids; borage seed CO₂-to extract; butter CO₂-se extract, dest. type; butter CO₂-se extract, cryst. type; chilli CO₂-to extract, 10% capsaicinoids; chilli CO₂-to extract, 5% capsaicinoids; Curcuma xanthorrhiza CO₂-se extract; dill seed CO₂-se extract, carvone type; dill seed CO₂-se extract, dillapiol type; peanut CO₂-se extract; fennel CO₂-se extract, bitter type; flavoxan 14, rosemary antioxidant formulation.

Foam inhibitors and defoamers; exemplary silicone oils with silica particles; mono- and diglycerides of fatty acids; polydimethylsiloxane (Simethicone®, E900); tri-n-butylphosphate; tri-isobutylphosphate; thermo-oxidised soybean oil esterified with MDG (mono- and diglycerides).

Examples of further suitable ingredients D for the topical pharmaceuticals, medical products and cosmetics according to the invention are known from: US 2012/0258150 A1, pages 4 to 6, TABLE 1, page 7, paragraph [0071], to page 9, paragraph [00 88] end; DE 10 2012 214 622 A1, page 4, paragraph [0016], to page 16, paragraph [0039]; 1 235 546 B1, page 7, paragraph [0016], to page 9, paragraph [0025] end, page 9, paragraphs [0027] to [0030], page 9, paragraph [0033], to page 14, [0055]; and DE 20 2014 010 286 U1, page 4, paragraph [0031], to page 8, paragraph [0066].

The topical pharmaceuticals, medical products and cosmetics according to the invention may contain conventional and known ionic and/or non-ionic surfactants as ingredients D (see Römpp Lexikon Lacke und Druckfarben, Georg Thieme Verlag, Stuttgart, 1998, keyword “Tenside”). However, it is a particular advantage of the topical pharmaceuticals, medical products and cosmetics according to the invention that in the vast majority of cases they manage without surfactants, in particular without ionic surfactants.

The proportion of the ingredients D in the topical pharmaceuticals, medical products and cosmetics according to the invention can vary very widely and can thus be excellently adapted to the respective intended use. Preferably, a given topical preparation contains 0.01% to 70% by weight, preferably 0.01% to 60% by weight and in particular 0.01% to 50% by weight of ingredients D, based on its total amount.

The topical pharmaceuticals according to the invention can contain further pharmaceuticals for special applications. Examples of suitable pharmaceuticals can be found in the PharmaiNDEX Yellow List.

Preferably, the topical pharmaceuticals, medical products and cosmetics according to the invention have a pH value between 3 and 7, preferably between 4.5-6.5 and in particular between 5-5.5, in particular in order not to damage the acid protection of the skin. In special cases, the topical pharmaceuticals, medical products and cosmetics according to the invention can also have a pH value >7.

The topical medical products and cosmetics according to the invention are preferably present as powders, solids, granules, gels, creams, ointments, lotions, drops, sprays, metered dose aerosols gargle solutions, lozenges, enemas, clysters, and coatings on and components of toiletries, textile fabrics, face masks, compresses, bandages, tampons, gauze, plasters, medical devices and release materials. This list is only exemplary and not exhaustive and other dosage forms may be considered, in particular for special applications.

In accordance with the invention, the topical pharmaceuticals according to the invention are strikingly prepared in the form of powders, solids, gels, creams, ointments, lotions, drops, sprays, metered dose aerosols, gargle solutions, lozenges, enemas, clysters, foams, release materials coatings and coating materials on and/or in release materials, sponges, textile fabrics, face masks, gauze and plasters as well as on and/or in roll-on pens, dispensers and medical devices. Again, this list is not exhaustive, but exemplary. Other preparations can also be used, particularly in special cases.

The topical pharmaceuticals according to the invention are prepared according to the method according to the invention. In this method, at least one type of particle A is mixed with at least one type of particle B and/or at least one type of particle C and the resulting mixture is homogenised. Subsequently, the resulting homogenised mixture (AB), (AC) or (ABC) is preferably processed into powders, solids, granules, gels, creams, ointments, lotions, drops, sprays, metered dose aerosols, gargle solutions, lozenges, enemas, clysters, suppositories or foams and/or applied to and/or in textile fabrics, face masks, gauze, compresses, bandages, tampons, plasters, medical devices and/or release materials.

In a preferred embodiment, at least one type of particles A is mixed with at least one type of particles B and/or at least one type of particles C, and/or at least one ingredient D, and the resulting mixture (ABC), (ABD), (ACD) or (ABCD), is homogenised, after which the homogenised mixture is processed as described above.

In a preferred embodiment, at least one type of particles A is mixed with at least one type of particles B and/or at least one type of particles C, at least one polyoxometalate (POM) and/or at least one ingredient D and the resulting mixture (ABC), (ABD), (AB POM), (ACD), (AC POM), (AD POM), (ABCD), (ABC POM) or (ABCD POM) is homogenised, after which the homogenised mixture is processed as described above.

When aqueous topical pharmaceuticals, medical products and cosmetics according to the invention are prepared according to the method of the invention, it proves to be advantageous if water containing carbon dioxide is used, because the carbon particles A are thereby better wetted. The carbon dioxide can be dissolved in the water, possibly working at overpressure, or blown or bubbled through the water.

For the method according to the invention, the usual and known plants, aggregates, apparatuses, dosing techniques and measuring techniques are used, as they are usually used in the fields of the preparation of cosmetics, care products, pharmaceuticals and medical aids and devices.

The topical medical products and cosmetics according to the invention can be used in a variety of ways. For example, they can be used as cosmetics, care products and products for saunas and steam baths and for the care of skin, in particular overlaps on the chest, abdomen and groin, mucous membranes, eyes, ears, mouth, lips, fingernails, toenails, cornea, warts and scars, with excretion of noxae, drugs, toxins, medication, acids, bases and odours via the skin, lesions, itching, intertrigo, burning, infections and redness, cancer therapy, chemotherapy, chondrocalcinosis, skin diseases such as psoriasis, neurodermatitis, acne, eczema, atrophic eczema, warts, rosacea, herpes, shingles, measles, mumps, rubella, chickenpox, hand-mouth-foot diseases, hand and foot syndrome, for preventive protection, to support healing processes in existing infections, wounds or burns, in decorative cosmetics, in cosmetic facial care, in hair care and in body care of humans and animals.

Preferably, the medical products according to the invention are used in the dosage form of powders, solids, granules, gels, creams, ointments, lotions, drops, sprays, metered dose aerosols, gargle solutions, lozenges, enemas, clysters, suppositories, foams and release materials as well as coatings on textile fabrics, face masks, compresses, bandages, tampons, gauze, plasters, toilet utensils and medical products. Again, this list is not exhaustive, but exemplary. Other product forms can also be used, particularly in special cases.

For example, the medical products and cosmetics according to the invention can be used in the appropriate dosage form
for hygiene, disinfection, risk of infection, fungal infections, including in the genital area, conjunctivitis, styes, dry eyes, skin rashes, itching, e.g. neurodermatitis, psoriasis, shingles, etc., to eliminate odours (such as sweat), to revitalise the skin, to soothe the skin, to cool the skin, for red, itchy, burning or swollen skin, for cracked, chapped skin (cracks), to protect against infection from bacteria, viruses, fungi, etc., after each shave, after insect bites, as a preventative measure, to support the healing process in the case of existing infections, wounds or burns, for mucous membranes, eyes, ears and mouth;
as medical cosmetics as concealer, as eye make-up products (other), as eyeliner, as eyeliner pencil, as body colour, as eye shadow, as lip make-up products (other), as lipstick sealant, as lipsticks, as make-up, as make-up remover, as nail whitener, as cuticle care/remover, as nail hardener, as nail glue remover, as nail polish remover, as nail polish thinner, as nail polish/nail make-up, as nail modelling agent, as mascara, as after andpre-shave products, as eye care products, as face masks, as face care products, as lip care products, as exfoliating products (chemical), as exfoliating products (mechanical), as exfoliating products (enzymatic), as shaving products (wet and dry shaving), as rinse-off products;
for medical hair care as anti-dandruff products, as bleaching agents for body hair, as perm products, as hair bleaching/decolouring agents, as hair dyes (non-oxidative), as hair dyes (oxidative), as hair setting agents (care), as hair setting agents (styling), hair straightening agents, as hair cleansing and hair care products (other), as hair styling products (other), as products against hair loss, as products for the care of the scalp and hair roots, as shampoos, as sun protection products for the hair;
for medical body care as antiperspirants, as bath and shower products, such as among others a mud bath, as bleaching agents for body hair, as depilatory products, as deodorants, as fragrance products (other), as epilation products, as foot care products, as hand care products, as skin bleaching products, as skin care products (other), as skin cleansing products (other), as intimate care products, as body care products, as nail care products, as perfumes and scented waters, as exfoliating products (chemical), as exfoliating products (mechanical), as pre- and after-sun products, as shaving products (wet and dry shaving), as soaps and syndets, as self-tanning products, as massage oil, as an alternative product to perfume, as self-tanning products, as sun protection products, as sun protection products for hair;
for medical dental and oral care: mouth sprays, mouthwashes, tooth whiteners, toothpastes.

A further field of application of the medical products according to the invention are applications for skin problems such as oxidative stress caused by radicals. Furthermore, various creams can be produced which are enriched during the day, for example with a UV filter, and at night with lipids. A regeneration balm can also be produced. A mucous membrane care and/or a scalp fleece can also be produced. Particularly preferably, sauna creams are used. Furthermore, creams that can be used in chemotherapy are particularly preferred. Furthermore, the products according to the invention do not trigger allergies and can even reduce existing allergies. In addition, the skin can be rejuvenated or the ageing process of the skin can be slowed down by the products according to the invention.

The topical pharmaceuticals according to the invention can be used in medicine in a variety of ways.

For example, the topical pharmaceuticals according to the invention can be used in the appropriate pharmaceutical form for the treatment of the diseases listed in Compilation 1.

Compilation 1: L00-L08 Infections of the skin and subcutaneous tissue

| Code | description | names of the diseases (synonyms) |
|---|---|---|
| L00 | Staphylococcal | Staphylococcal scalded skin syndrome (Dermatitis exfoliativa |
| | scalded skin | neonatorum, Pemphigus acutus neonatorum) |
| | syndrome | |
| L01 | Impetigo | Impetigo contagiosa, Folliculitis superficialis, Impetiginisation |
| L02 | Skin abscess, | Skin abscess, furuncle (furunculosis), carbuncle (boil) |
| | furuncle and | |
| | carbuncle | |
| L03 | Phlegmon | Phlegmon, Acute Lymphangitis, nail infection (onychia, paronychia, |
| | | perionychia), facial phlegmon, omphalitis, cellulitis |
| L04 | Acute | Acute lymphadenitis, lymph node abscess |
| | Lymphadenitis | |
| L05 | Pilonidal | Pilonidal cyst (pilonidal fistula, pilonidal sinus, coccygeal fistula, |
| | cyst | coccygeal cyst) |
| L06 | Not assigned | — |
| L07 | | |
| L08 | Other localised | Pyoderma, dermatitis purulenta, dermatitis septica, dermatitis |
| | infections of the | suppurativa, erythrasma |
| | skin and subcutis | |

L10-L14 Bullous Dermatoses

| Code | description | Names of the diseases (synonyms) |
|---|---|---|
| L10 | Pemphigus diseases | Pemphigus vulgaris, Pemphigus vegetans, Pemphigus |
| | | foliaceus, Brazilian pemphigus (Fogo selvagem), Pemphigus |
| | | erythematosus, Senear-Usher syndrome, Drug-induced |
| | | pemphigus |
| L11 | Other acantholytic | Acquired keratosis follicularis, transient acantholytic |
| | dermatoses | dermatosis (Grover's transient acantholytic dermatosis) |
| L12 | Pemphigoid diseases | Bullous pemphigoid, Cicatricial pemphigoid, Benign mucous |
| | | membrane pemphigoid, Chronic bullous dermatosis of |
| | | childhood, Acquired epidermolysis bullosa, Linear IgA |
| | | dermatosis |
| L13 | Other bullous | Dermatitis herpetiformis (Dermatitis herpetiformis Duhring), |
| | dermatoses | Pustulosis subcornealis (Pustulosis subcornealis Sneddon- |
| | | Wilkinson) |
| L14* | Bullous dermatoses in | — |
| | diseases classified | |
| | elsewhere | |

L20-L30 dermatitis and eczema

| Code | description | Names of the diseases (synonyms) |
|---|---|---|
| L20 | Atopic (endogenous) | Atopic eczema (endogenous eczema, allergic eczema, |
| | eczema | intrinsic eczema, endogenous cradle cap, neurodermatitis, |
| | | neurodermatitis atopica, neurodermatitis diffusa), Prurigo |
| | | Besnier |
| L21 | Seborrhoeic eczema | Seborrhoeic eczema, seborrhoea capitis, seborrhoeic cradle |
| | | cap, seborrhoeic eczema of children |
| L22 | Nappy dermatitis | Nappy dermatitis, psoriasiform nappy dermatitis, nappy |
| | | rash, nappy erythema |
| L23 | Allergic contact dermatitis | Allergic contact dermatitis (allergic contact dermatitis), |
| | | nickel allergy |
| L24 | Toxic contact dermatitis | Toxic contact dermatitis (non-allergic contact dermatitis, |
| | | toxic contact dermatitis, irritant contact dermatitis), acne |
| | | venenata (contact acne), chloracne, oil acne |
| L25 | Unspecified contact | Baker's scabies |
| | dermatitis | |
| L26 | Exfoliative dermatitis | Exfoliative dermatitis, pityriasis rubra (pityriasis rubra |
| | | hebra) |
| L27 | Dermatitis caused by | — |
| | orally, enterally or | |
| | parenterally ingested | |
| | substances | |
| L28 | Lichen simplex chronicus | Lichen simplex chronicus (lichen vidal), neurodermatitis |
| | and prurigo | chronica circumscripta, prurigo nodularis, prurigo, prurigo |
| | | hebra, prurigo mitis, urticaria papulosa |
| L29 | Pruritus | Pruritus ani, pruritus scrotalis, pruritus vulvae, pruritus |
| | | anogenitalis |
| L30 | Other dermatitis | Nummular eczema, Dyshidrosis (pompholyx), Candida |
| | | mycid (levurid), Dermatophytid, Eczematid, Eczematoid |
| | | dermatitis, Infectious dermatitis, Superinfected eczema, |
| | | Intertriginous eczema, Pityriasis alba faciei |

L40-L45 Papulosquamous skin diseases

| Code | description | Names of the diseases (synonyms) |
|---|---|---|
| L40 | Psoriasis | Psoriasis vulgaris, Psoriasis nummularis, Generalised pustular |
| | | psoriasis, Impetigo herpetiformis, Psoriasis pustulosa |
| | | Zumbusch, Acrodermatitis continua suppurativa |
| | | (acrodermatitis continua suppurativa Hallopeau), Psoriasis |
| | | pustulosa palmoplantaris, Psoriasis guttata, Psoriasis |
| | | arthropathy, Psoriasis inversa |
| L41 | Parapsoriasis | pityriasis lichenoides et varioliformis acuta (Pityriasis |
| | | lichenoides et varioliformis acuta Mucha-Habermann), |
| | | Parapsoriasis guttata, Papulosis lymphomatoides, Small |
| | | patchy parapsoriasis en plaques, Large patchy parapsoriasis en |
| | | plaques, Parapsoriasis with poikiloderma |
| L42 | Pityriasis rosea | Pityriasis rosea (pink lichen) |
| L43 | Lichen ruber planus | Lichen ruber hypertrophicus, Lichen ruber pemphigoides, |
| | | Lichenoid drug reaction, Subacute lichen ruber planus, Lichen |
| | | planus tropicus |
| L44 | Other papulosquamous | Pityriasis rubra pilaris, Lichen nitidus, Lichen striatus, Lichen |
| | skin diseases | ruber moniliformis, Infantile papular acrodermatitis (Gianotti- |
| | | Crosti syndrome) |
| L45* | Papulosquamous skin | — |
| | diseases in diseases | |
| | elsewhere classified | |

L50-L54 Urticaria and Erythema

| Code | description | Names of the diseases (synonyms) |
|---|---|---|
| L50 | Urticaria | Allergic urticaria, idiopathic urticaria, cold urticaria, heat urticaria, |
| | | urticaria factitia, urticarial dermographism, urticaria mechanica, |
| | | cholinergic urticaria, contact urticaria, chronic urticaria, recurrent |
| | | urticaria, periodic urticaria |
| L51 | Erythema | Non-bullous erythema exsudativum multiforme (erythema |
| | exsudativum | exsudativum multiforme minus), bullous erythema exsudativum |
| | multiforme | multiforme (erythema exsudativum multiforme majus, Stevens- |
| | | Johnson syndrome), toxic epidermal necrolysis (Lyell syndrome) |
| L52 | Erythema nodosum | Erythema nodosum |
| L53 | Other | Erythema toxicum, erythema anulare centrifugum, erythema |
| | erythematous | marginatum, figured chronic erythema, erythroderma |
| | diseases | |
| L54* | Erythema in | Erythema marginatum in acute rheumatic fever |
| | diseases classified | |
| | elsewhere | |

L55-L59 Diseases of the skin and subcutis due to exposure to radiation

| Code | description | Names of the diseases (synonyms) |
|---|---|---|
| L55 | Dermatitis solaris acuta | Dermatitis solaris acuta (sunburn) |
| L56 | Other acute skin changes | Phototoxic reaction to drugs, Photoallergic reaction to |
| | due to ultraviolet rays | drugs, Phototoxic contact dermatitis, Berloque dermatitis, |
| | | Urticaria solaris, Polymorphic light dermatosis |
| L57 | Skin changes due to | Actinic keratosis, (keratosis senilis, keratosis solaris), |
| | chronic exposure to non- | actinic reticuloid, cutis rhomboidalis nuchae, poikilodermia |
| | ionising radiation | reticularis (poikilodermia reticularis Civatte), cutis laxa |
| | | senilis (senile actinic elastosis, elastosis senilis), radiation |
| | | granuloma, landman's skin, sailor's skin |
| L58 | Radiodermatitis | acute radiodermatitis, chronic radiodermatitis |
| L59 | Other diseases of the skin | Erythema ab igne, Chronic heat injury |
| | and subcutaneous tissue | |
| | due to exposure to | |
| | radiation | |

L60-L75 Diseases of skin appendages

| Code | description | Names of the diseases (synonyms) |
|---|---|---|
| L60 | Diseases of | Unguis incarnatus (ingrown nail), onycholysis, onychogryposis |
| | the nails | (onychogryphosis), nail dystrophy, Beau-Reil transverse furrows, yellow |
| | | nail syndrome (yellow nail syndrome) |
| L61 | not assigned | — |
| L62* | Diseases of | — |
| | the nails in | |
| | diseases | |
| | classified | |
| | elsewhere | |
| L63 | Alopecia | Alopecia cranialis totalis (alopecia totalis), alopecia universalis, ophiasis |
| | areata | |
| L64 | Alopecia | -type alopecia, Drug-induced alopecia androgenetica |
| | androgenetica | |
| L65 | Other hair | Telogen effluvium, anagen effluvium, alopecia mucinosa pinkus (alopecia |
| | loss without | mucinosa) |
| | scarring | |
| L66 | Cicatricial | Pseudopelade Brocq, Lichen planopilaris, Lichen ruber follicularis, |
| | alopecia | Folliculitis decalvans, Folliculitis et Perifolliculitis capitis abscedens et |
| | (hair loss | suffodiens Hoffmann (Folliculitis et Perifolliculitis capitis abscedens et |
| | with | suffodiens), Atrophodermia vermiculata, Folliculitis ulerythematosa |
| | scarring) | reticulata, Ulerythema acneiforme |
| L67 | Abnormalities | Trichorrhexis nodosa, canities, premature greying, heterochromia of hair, |
| | of hair | poliosis, acquired poliosis circumscripta, fragilitas crinium |
| | colour and | |
| | hair growth | |
| L68 | Hypertrichosis | Increased hair growth, hirsutism, hypertrichosis lanuginosa acquisita, |
| | | localised hypertrichosis, polytrichia |
| L69 | not assigned | — |
| L70 | Acne | Acne vulgaris, Acne conglobata, Acne varioliformis, Acne necroticans |
| | | miliaris, Acne tropica, Acne infantum, Acne excoriée des jeunes filles |
| L71 | Rosacea | perioral dermatitis, rhinophyma |
| L72 | Follicular | Epidermal cyst, trichilemmal cyst, atheroma, pilar cyst, steatocystoma |
| | cysts of skin | multiplex |
| | and subcutis | |
| L73 | Other | Acne keloid (folliculitis sclerotisans nuchae), pseudofolliculitis barbae, |
| | diseases of | hidradenitis suppurativa, folliculitis barbae |
| | the hair | |
| | follicles | |
| L74 | Diseases of | Miliaria rubra, Miliaria cristallina, Miliaria profunda, Miliaria tropica, |
| | eccrine | Anhidrosis, Hypohidrosis |
| | sweat glands | |
| L75 | Diseases of | Bromhidrosis, chromhidrosis, apocrine miliaria, Fox-Fordyce disease |
| | apocrine | |
| | sweat glands | |

L80-L99 Other diseases of the skin and subcutis

| Code | description | Names of the diseases (synonyms) |
|---|---|---|
| L80 | Vitiligo | Vitiligo |
| L81 | Other disorders of skin | Postinflammatory hyperpigmentation, chloasma (melasma), |
| | pigmentation | ephelides (freckles), café-au-lait spots, lentigo, leukoderma, |
| | | purpura, angioma serpiginosum, essential |
| | | pigment purpura, angioma serpiginosum, essential |
| | | telangiectasia, tattooing |
| L82 | Seborrhoeic keratosis | Seborrhoeic keratosis, dermatosis papulosa nigra, Leser- |
| | | Trélat syndrome |
| L83 | Acanthosis nigricans | Acanthosis nigricans, papillomatosis confluens et reticularis |
| | | (papillomatosis confluens et reticularis Gougerot-Carteaud) |
| L84 | Corns and cornea Corns | callus, callus, clavus |
| L85 | Other epidermal | Acquired ichthyosis, Acquired keratosis palmoplantaris |
| | thickening | (Acquired keratoma palmoplantare), Keratosis punctata |
| | | (Keratosis palmoplantaris), Xerosis cutis (Xeroderma), |
| | | Cornu cutaneum, Epidermal thickening |
| L86* | Keratoma in diseases | — |
| | classified elsewhere | |
| L87 | Disorders of | hyperkeratosis follicularis et parafollicularis in cutem |
| | transepidermal | penetrans (hyperkeratosis follicularis et parafollicularis in |
| | elimination | cutem penetrans Kyrle, hyperkeratosis follicularis |
| | | penetrans), reactive perforating collagenosis, elastosis |
| | | perforans serpiginosa |
| L88 | Pyoderma gangraenosum | Pyoderma gangraenosum, ulcerative dermatitis, phagedenic |
| | | pyoderma |
| L89 | Decubital ulcer | Decubitus ulcer, pressure ulcer, ulcer |
| L90 | Atrophic skin diseases | Lichen sclerosus et atrophicus, anetoderma, atrophodermia |
| | | idiopathica, acrodermatitis chronica atrophicans |
| | | Herxheimer, scars and fibrosis of the skin, striae cutis |
| | | atrophicae |
| L91 | Hypertrophic skin | Hypertrophic scar, cicatricial keloid |
| | diseases | |
| L92 | Granulomatous diseases | Granuloma anulare (granuloma anulare perforans), |
| | of the skin and subcutis | necrobiosis lipoidica, granuloma faciale, (granuloma |
| | | eosinophilicum faciei), foreign body granuloma |
| L93 | Lupus erythematosus | Discoid lupus erythematosus, subacute lupus erythematosus |
| | | cutaneus, lupus erythematosus profundus, lupus panniculitis |
| L94 | Other localised diseases | Sclerodermia circumscripta (morphaea, localised |
| | of connective tissue | scleroderma), linear scleroderma (band-shaped scleroderma, |
| | | sclérodermie en coup de sabre), calcinosis cutis, |
| | | sclerodactyly, Gottron papules, poikilodermia atrophicans |
| | | vascularis (poikilodermia atrophicans vascularis Jacobi), |
| | | ainhum |
| L95 | Vasculitis not elsewhere | Livedo vasculitis, capillaritis alba, erythema elevatum et |
| | classified, limited to the | diutinum |
| | skin | |
| L96 | not assigned | — |
| L97 | Ulcus cruris, not | Ulcus cruris |
| | elsewhere classified | |
| L98 | Other diseases of the skin | Granuloma pediculatum (granuloma pyogenicum), |
| | and subcutaneous tissue, | Dermatitis factitia (neurotic excoriation), Acute febrile |
| | not elsewhere classified | neutrophilic dermatosis (Sweet syndrome), Eosinophilic |
| | | cellulitis (Wells syndrome), Ulcus tropicum, Mucinosis of |
| | | the skin, Focal mucinosis, Lichen myxoedematosus |
| L99* | Other diseases of the skin | Cutaneous amyloidosis (lichen amyloidosus, macular |
| | and subcutaneous tissue | amyloidosis) |
| | in diseases classified | |
| | elsewhere | |

Further indications for the topical pharmaceuticals according to the invention, possibly already mentioned above, are skin fungi, nail fungi and discolouration and nail bed inflammation, acne, hair loss, herpes, allergic rhinitis, rhinitis, hives, vaginal fungus, skin burns, severe allergic skin reactions, inflammation and replacement of Voltaren and other painkillers for knee discomfort, arthrosis, gonoarthrosis, hip dicomfort, polyarthritis and ankylosing spondylitis.

Further indications for the topical pharmaceuticals according to the invention are rheumatic diseases, which include very different clinical pictures that are categorised into four main groups according to their cause. A further subdivision is made within these groups. The following classification scheme currently applies (see compilation 2). Here, the topical pharmaceutical according to the invention is used for cortisone replacement treatment.

### Compilation 2: Rheumatic Diseases

Inflammatory rheumatic diseases (autoimmune):
Rheumatoid arthritis (chronic polyarthritis)
rheumatic fever is clearly distinguished form that (also known as acute rheumatoid arthritis, polyarthritis rheumatica acuta and rheumatism acutus verus) since 1953
Juvenile idiopathic arthritis
Spondylarthritis
Ankylosing spondylitis (Bechterew's disease)
Psoriatic arthritis
Enteropathic arthritis (concomitant with intestinal diseases such as ulcerative colitis)
Reactive arthritis
Undifferentiated spondyloarthritis
Collagenoses (connective tissue diseases):
Lupus erythematosus
Systemic sclerosis
Sjögren's syndrome
Polymyositis and dermatomyositis
Mixed collagenosis
asculitides (diseases with vascular inflammation)
Polymyalgia rheumatica
Degenerative (“wear-related”) rheumatic diseases:
Osteoarthritis
tendonitis
Metabolic disorders associated with rheumatic complaints:
Gout and other crystal deposition diseases
Haemochromatosis (iron metabolism disorder)
Rheumatic non-inflammatory diseases of the soft tissues with symptoms such as pain in the muscles and tendons:
Fibromyalgia (non-inflammatory soft tissue rheumatism, “soft tissue rheumatism”)
Rheumatism-like diseases

The heavy metal-free topical pharmaceuticals according to the invention for use in medicine have in some variants specific inner surface areas of the activated carbon particles (A) according to BET <200 m²/g.

Furthermore, the heavy metal-free topical medicaments for use in medicine according to the invention do not contain any organically bound halogens.

The activated carbon particles (A) of the present invention have active surfaces with a high degree of adsorption. This was particularly surprising because the activated carbon particles (A) of the present invention no longer have an open-pored structure, as they are free of meso- and macropores.

This is also surprising in view of the fact that in the particle size range of the activated carbon particles (A) of the present invention—i.e. a particle size determined by electron microscopy in the range from 50 nm to 1000 nm or from 100 nm to 1000 nm—they should actually behave like graphite. Surprisingly, however, this is not the case; the surfaces of the activated carbon particles (A) according to the present invention exhibit high adsorption properties.

Corresponding particles with such properties and characteristics were not known from the prior art.

The same applies to the activated carbon particles (A) prepared with the mechanochemical process for preparing activated carbon particles (A) according to the invention.

A major advantage of the activated carbon particles (A) according to the invention or of the activated carbon particles (A) prepared by the mechanochemical process according to the invention for the preparation of activated carbon particles (A) is that, due to the specific particle size distribution of 50 nm to 1000 nm or 100 nm to 1000 nm, preferably 200 nm to 900 nm, more preferably 300 nm to 800 nm, in particular preferably 400 nm to 700 nm, as well as in other variants preferably from 600 nm to 680 nm, preferably 620 nm to 660 nm and in particular 640 nm+/−5 nm, it allows the activated carbon particles (A) to penetrate into follicles, in particular ovarian follicles, hair follicles, dental follicles, lymphatic follicles and/or thyroid follicles.

In preferred variants of the present invention, the activated carbon particles (A) are used for the preparation of or in creams, wherein the cream formulation particularly preferably does not contain lipids, since these could themselves adsorb to the activated carbon particles (A).

It is surprising in the context of the present invention that creams based on the activated carbon particles (A) according to the invention exhibit excellent property profiles even without the use of lipids. The same applies to the non-topical or topical pharmaceuticals, medical products and cosmetics according to the invention, in particular free of heavy metals, especially if these are formulated as creams. For these, too, it is correspondingly preferred if creams are formulated which are formulated without lipids.

Particularly preferred in variants of the present invention are thus:
Uses of the activated carbon (A) according to the present invention or the activated carbon (A) prepared by the mechanochemical process according to the present invention as a component of lipid-free formulated creams or corresponding creams per se;
heavy metal-free topical pharmaceuticals, medical products and/or cosmetics, which are preferably present as creams, particularly preferably as lipid-free formulated creams;
methods for the preparation of heavy metal-free topical pharmaceuticals, medical products and cosmetics according to the present invention, wherein the topical pharmaceuticals, medical products and cosmetics are preferably prepared as creams, particularly preferably as lipid-free formulated creams;
the use of the heavy metal-free topical pharmaceuticals, medical products and cosmetics preferably in the form of creams, particularly preferably lipid-free formulated creams.

The activated carbon particles (A) according to the present invention or the compositions based thereon, in particular lipid-free creams (for the sake of simplicity, in the following list reference is made to products according to the invention), have various advantageous properties or functions. Only exemplary of few are to be mentioned:
the products have an anti-inflammatory effect, in particular because inflammatory factors can be reduced by binding them by means of adsorption; this applies, for example, to prostaglandins;
the products according to the invention have a toxin-eliminating effect on the skin, e.g. toxins from nettles (e.g. stinging nettles or jellyfish) or toxins from oak processionary moths are eliminated (it is assumed, without being bound to this assumption, that the toxins adsorb on the activated carbon particles (A));
the products according to the invention are also surprisingly capable of eliminating toxins that have already penetrated into the skin (it is assumed, without being bound to this assumption, that this is made possible by shifting the chemical equilibrium);
the products according to the invention are capable of eliminating or reducing toxins in the skin which are present in follicles and/or the dermis (it is assumed, without being bound to this assumption, that this is done by shifting the chemical equilibrium in adsorption and absorption)—preferred examples of such toxins are chemotherapeutic agents and their degradation products;
the products of the present invention are capable of significantly reducing or completely eliminating itching;
the products according to the present invention are able to reduce swellings, such as those occurring in gout, rheumatism, etc.

The products according to the invention can be used in a wide variety of ways, as is apparent to the skilled person when considering the rest of the description and the stated actions and effects. In addition to the possible applications already mentioned above, the following possible applications should be mentioned explicitly (additionally or again):
use in diseases and applications as they arise on the basis of the stated effects (i.e. if the beneficial effects contribute/lead to relief and/or cure),
as (topical) pharmaceuticals,
as (topical) medical products,
as (topical) cosmetics (e.g. also as cosmetics with a promise of efficacy).

### FIGURES

FIGS. 1 to 19 are schematic representations intended to illustrate the essential features of the mechanochemical process according to the invention and its use according to the invention. Therefore, the figures are partly not drawn to scale:

FIG. 1 a mechanical mill 1 according to the invention with fixed drum 1.5 with attritor 1.4 for grinding material to be ground F;

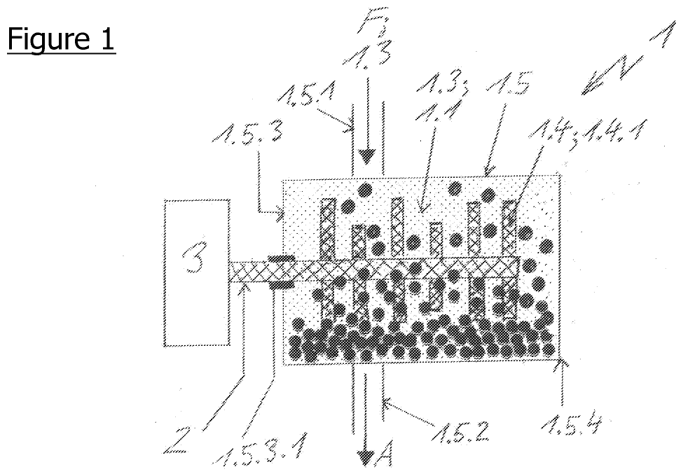

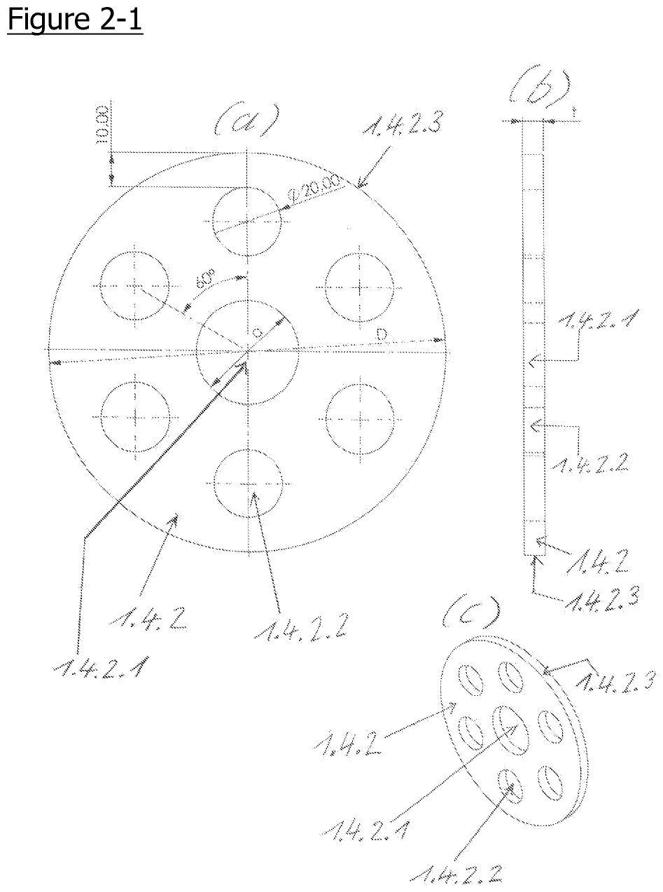

FIG. 2-1 (a) top view of the true-to-scale surface of the impact disc 1.4.2,
(b) true-to-scale side view of the impact disc 1.4.2 and
(c) perspective view of the impact disc 1.4.2;

FIG. 2-2 (a) top view of the true-to-scale surface of the impact disc 1.4.2,
(b) true-to-scale side view of the impact disc 1.4.2 and
(c) perspective view of the impact disc 1.4.2;

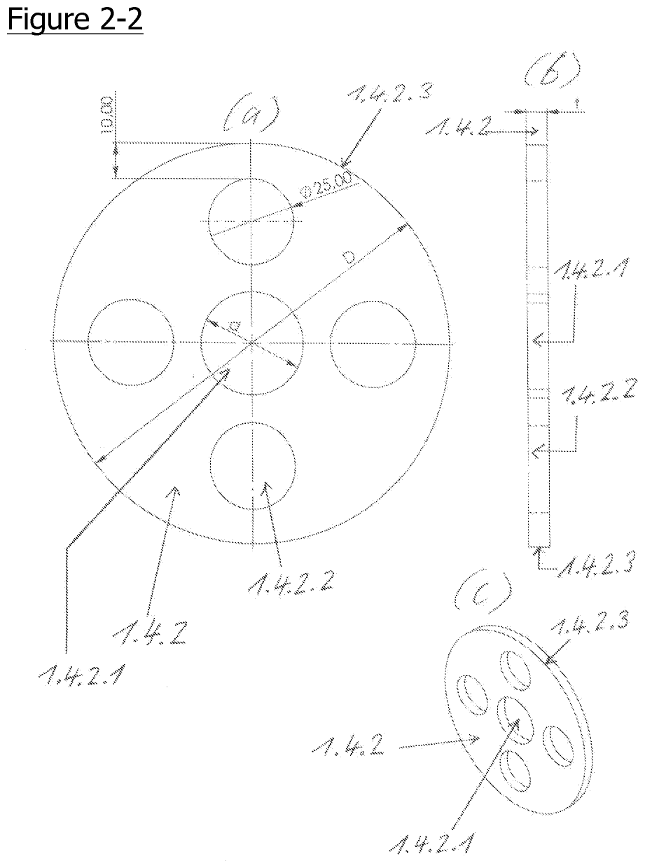

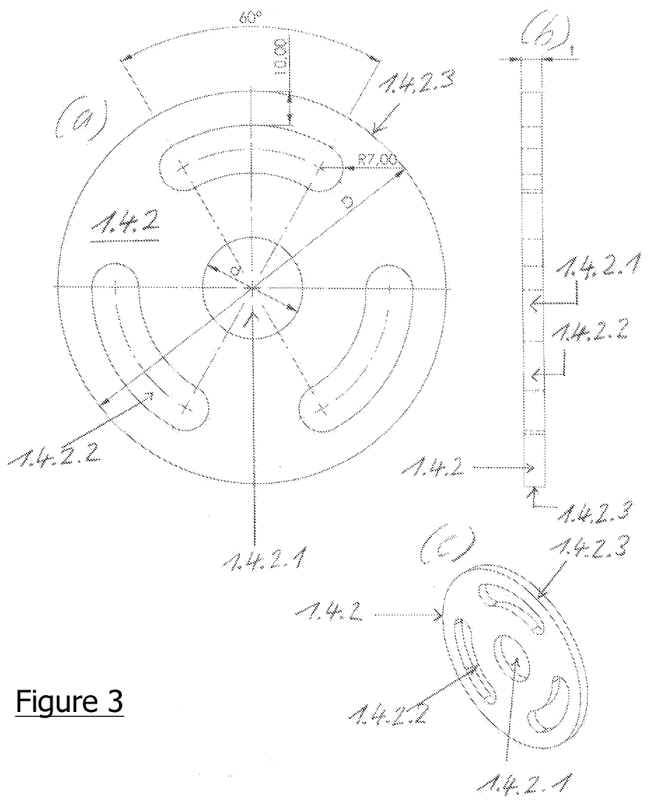

FIG. 3 (a) top view of the true-to-scale surface of the impact disc 1.4.2,
(b) true-to-scale side view of the impact disc 1.4.2 and
(c) perspective view of the impact disc 1.4.2;

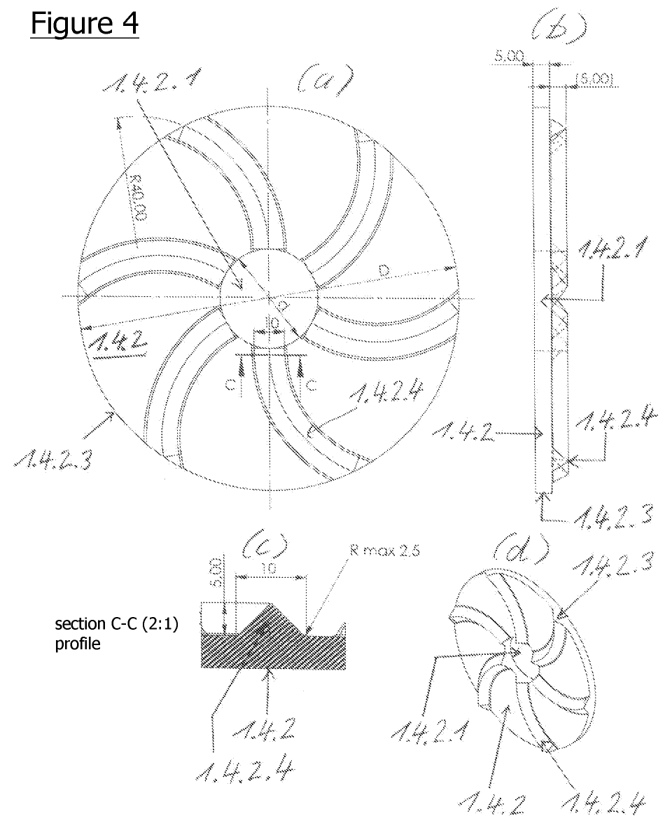

FIG. 4 (a) top view of the true-to-scale surface of the impact disc 1.4.2 with the impact webs 1.4.2.4,
(b) true-to-scale side view of the impact disc 1.4.2,
(c) profile of an impact web 1.4.2.4 and
(d) perspective view of the impact disc 1.4.2;

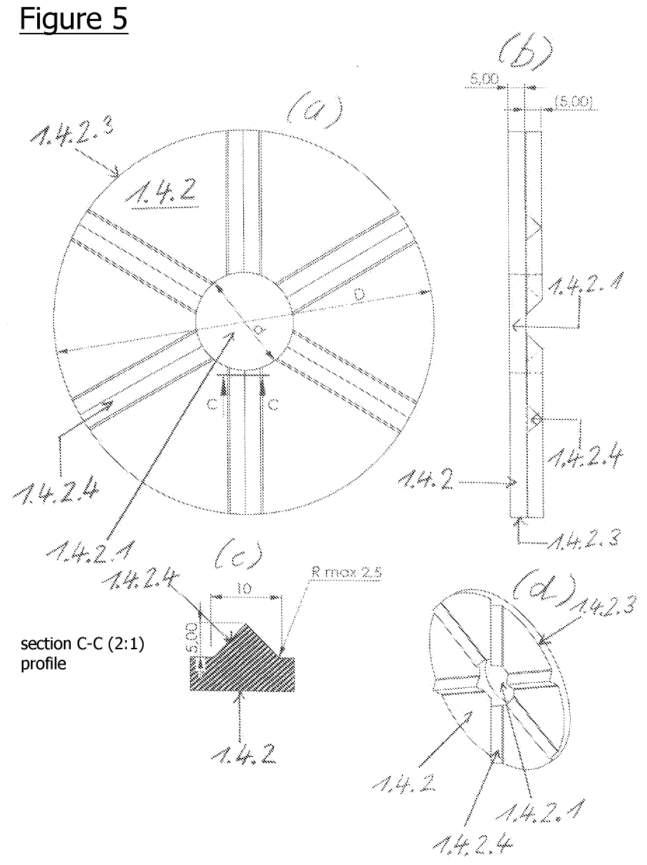

FIG. 5 (a) top view of the true-to-scale surface of the impact disc 1.4.2 with the impact webs 1.4.2.4,
(b) true-to-scale side view of the impact disc 1.4.2,
(c) profile of an impact web 1.4.2.4 and
(d) perspective view of the impact disc 1.4.2;

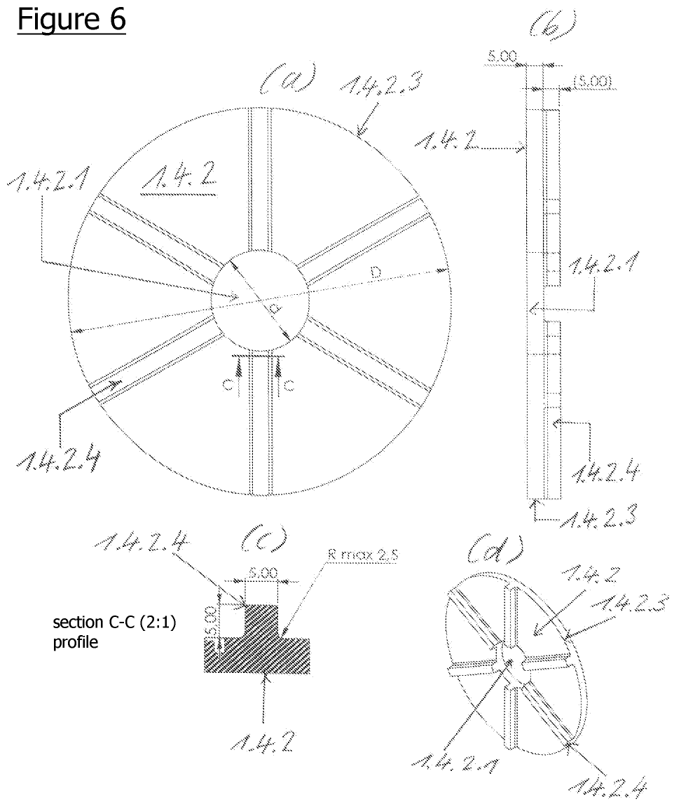

FIG. 6 (a) top view of the true-to-scale surface of the impact disc 1.4.2 with the impact webs 1.4.2.4,
(b) true-to-scale side view of the impact disc 1.4.2,
(c) profile of an impact web 1.4.2.4 and
(d) perspective view of the impact disc 1.4.2;

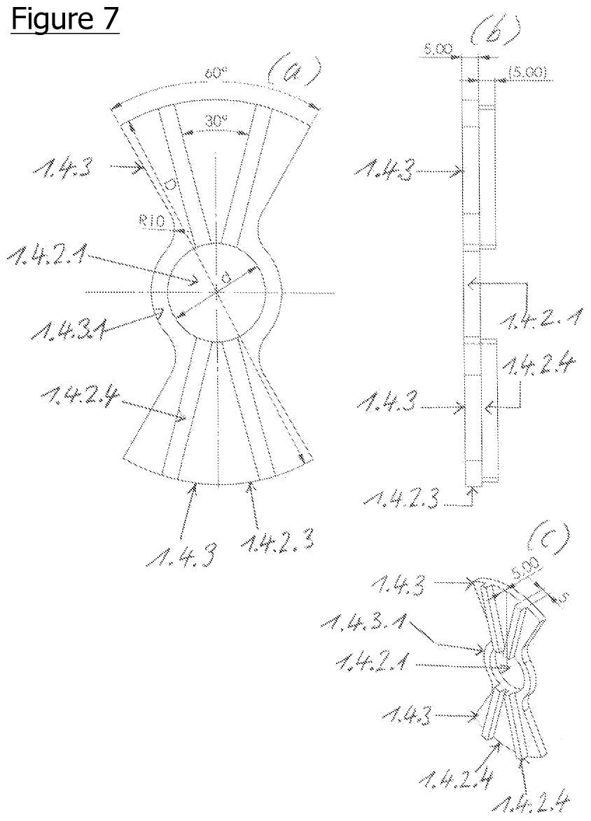

FIG. 7 (a) top view of the true-to-scale surface of the impact fan 1.4.3 with the impact webs 1.4.2.4,
(b) true-to-scale side view of the impact fan 1.4.2 and
(c) perspective view of the impact fan 1.4.2;

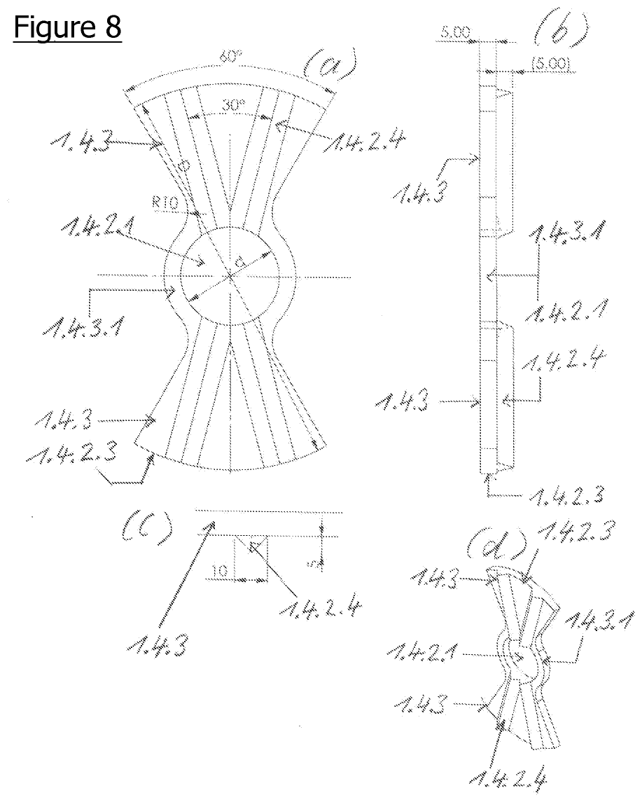

FIG. 8 (a) top view of the true-to-scale surface of the impact fan 1.4.3 with the impact webs 1.4.2.4,
(b) true-to-scale side view of the impact fan 1.4.2,
(c) profile of an impact web 1.4.2.4 and
(d) perspective view of the impact fan 1.4.2;

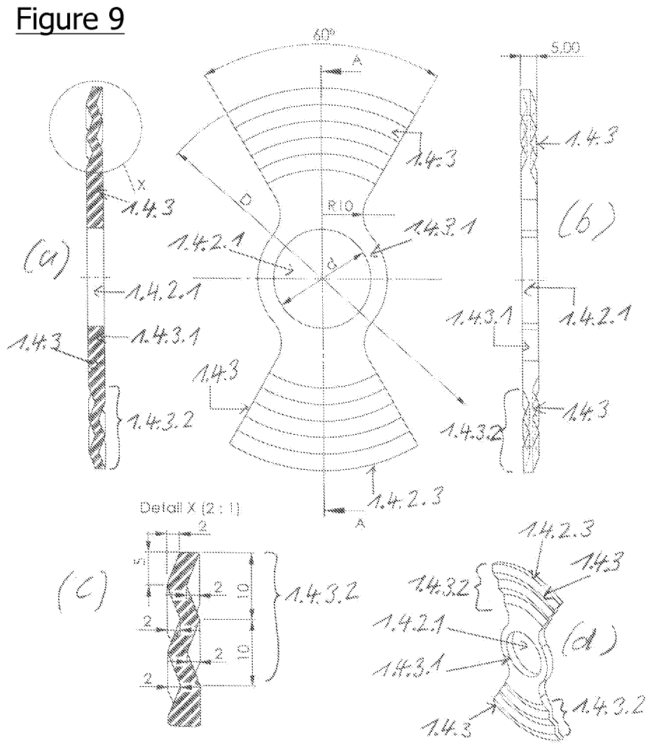

FIG. 9 top view of the true-to-scale surface of the impact fan 1.4.3 with the impact webs 1.4.2.4,
(a) and (b) true-to-scale side views of the impact fan 1.4.2,
(c) profile of an impact web 1.4.2.4 and
(d) perspective view of the impact fan 1.4.2;

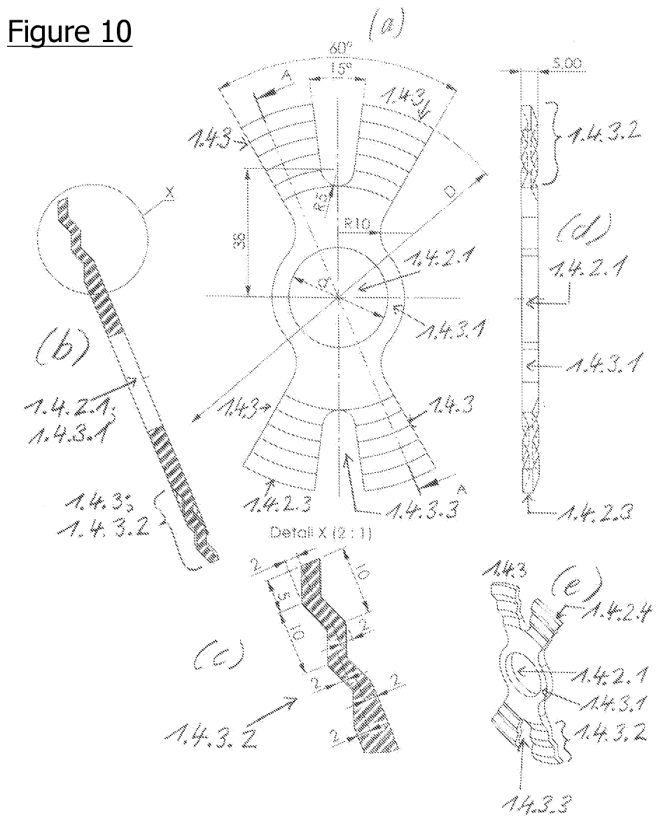

FIG. 10 (a) top view of the true-to-scale surface of the double impact fan 1.4.3 with the crest and trough profiles 1.4.3.2 and the U-shaped gaps 1.4.3.3, (b) and (d) true-to-scale side views of the double impact fan 1.4.3, (c) section through the crest and trough profile 1.4.3.2 and (e) perspective view of the double impact fan 1.4.3;

FIG. 11 top view of an impact lobe 1.4.4 with symmetrically arranged impact bodies 1.4.4.3;

FIG. 12 top view of a impact blade 1.4.5;

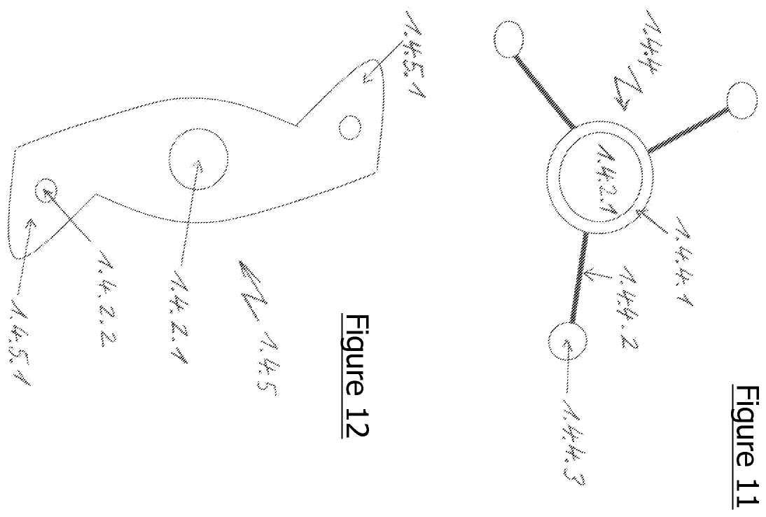

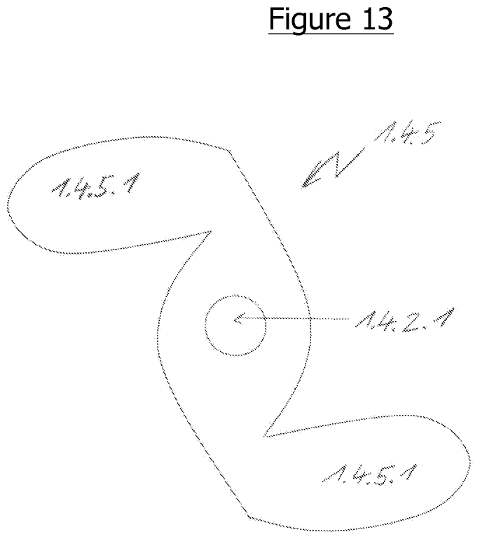

FIG. 13 top view of a further embodiment of the impact blade 1.4.5;

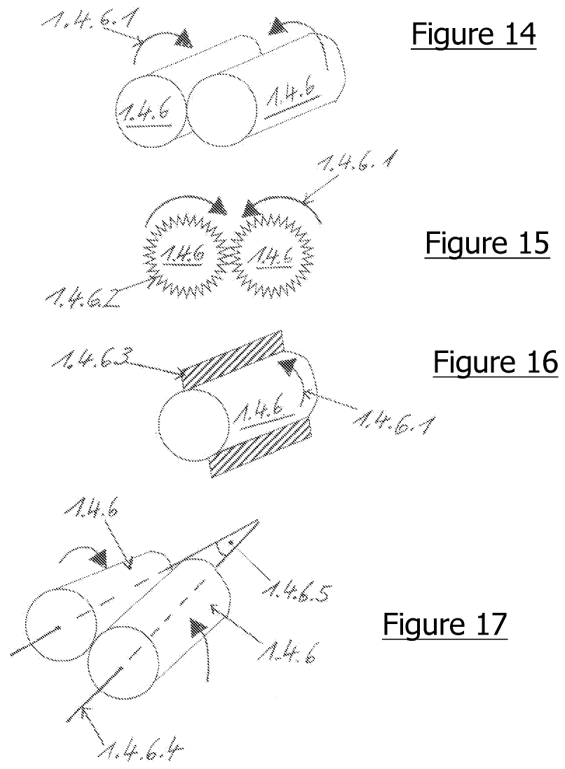

FIG. 14 grinding with two counter-rotating rollers 1.4.6;

FIG. 15 grinding with two counter-rotating rollers 1.4.6 with surface structures 1.4.6.2;

FIG. 16 grinding with a roller 1.4.6 and an abrasion surface 1.4.6.3;

FIG. 17 grinding with two counter-rotating rollers 1.4.6 inclined towards each other at a certain angle 1.4.6.5;

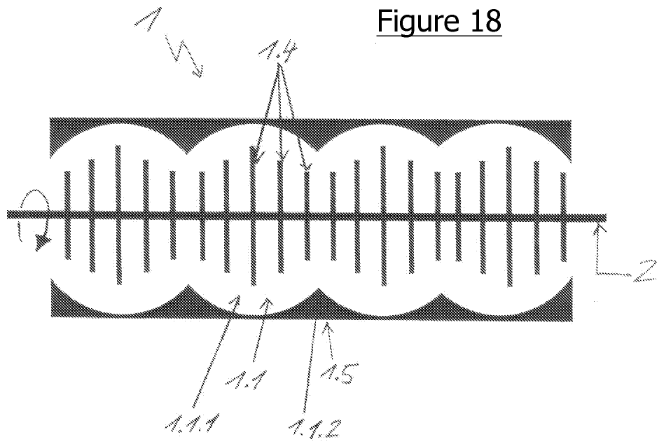

FIG. 18 drum 1.5 comprising several grinding chambers 1.1.1;

In FIGS. 1 to 19, the reference signs have the following meaning:
1 mechanical mill, grinding unit
1.1 mechanical reactor, grinding chamber
1.1.1 spherical grinding chamber
1.1.2 circular constriction
1.2 grinding media
1.3 material to be ground F
1.4 agitation means
1.4.1 attritor
1.4.2 impact disc
1.4.2.1 passage for the drive shaft 3
1.4.2.2 impact hole
1.4.2.3 edge
1.4.2.4 impact web
1.4.3 impact fan
1.4.3.1 ring around the drive shaft 3
1.4.3.2 crest and trough profile
1.4.3.3 U-shaped gap
1.4.4 impact lobe
1.4.4.1 ring around the drive shaft 3
1.4.4.2 connecting web
1.4.4.3 impact body
1.4.5 impact blade
1.4.5.1 impact end
1.4.6 rotating roller
1.4.6.1 direction of rotation
1.4.6.2 tooth
1.4.6.3 abrasion surface
1.4.6.4 axis of rotation
1.4.6.5 angle of inclination
1.4.6.6 roller surface
1.4.6.7 recess
1.4.6.8 springing
1.4.6.9 ball
1.5 drum
1.5.1 inlet for material to be ground F; 1.3
1.5.2 outlet for ground product A
1.5.3 disc-shaped, vertical drum wall
1.5.3.1 passage through 1.5.3
1.5.4 1.5.3 opposite drum wall
2 drive shaft
2.1 direction of rotation
3 motor

In the following, the present invention is explained in more detail by means of examples and comparative tests. The examples and comparative tests serve to illustrate the invention and are not limiting.

### EXAMPLES

### Examples Group I

In the following example text, the abbreviated designations have the following meaning:
A activated carbon particles according to the invention
F material to be ground

In the following, it is no longer mentioned in detail that all surfaces inside the grinding chamber 1.1 that come into contact with the material to be ground F were coated with an aluminium oxide ceramic.

### Reference Example 1-I

The grinding units 1 with attritors 1.4.1 described in the German publication DE 195 04 540 A1 were used for the grinding tests of the reference example. As grinding media 1.2, 2000 steel balls weighing 1 g each were used. The weight ratio of the weight of material to be ground F:weight of the ceramic balls 1.2 was 1:10. The grinding tests were carried out under nitrogen at atmospheric pressure.

As drive of the grinding unit 1 with attritors 1.4.1 an electric motor 3 according to the international patent application WO 2017/055246 A2 (as described above) was used.

### Reference Example 2-I

### Mechanical Mills for Carrying Out the Mechanochemical Process According to the Invention

The mechanical mill 1 for mechanochemical processes comprises at least one stationary mechanochemical reactor 1.1 (FIG. 1), which comprises a plurality of balls made of technical ceramics as grinding media 1.2 in a drum 1.5 made of stainless steel lined with technical ceramics and having at least one inlet 1.5.1 for the material to be ground F; 1.3 and at least one outlet 1.5.2 for the ground product I. The technical ceramic used is aluminium oxide ceramic (see Ulrike Wiech in the company publication of Ceram Tec-ETEC GmbH, Lohmar, think ceramics TECHNISCHE KERAMIK, pages 211 and 212, 3.4.4.2 Mahlen und Brechen).

The drum 1.5 of the stationary mechanochemical reactor 1.1 of FIG. 1 has a stainless steel attritor 1.4 coated with the technical ceramic for mixing the grinding media 1.2 and the material to be ground 1.3. The attritor is arranged rotatably along the longitudinal axis of the drum 1.5 and is rotated by a rotatable drive shaft 2 driven by a motor 3 and passing through the passage 1.5.3.1 passing through the disc-shaped vertical drum wall 1.5.3.

The drum 1.5 (FIG. 1) can also have the shape shown in FIGS. 1a, 1b, 2a and 3a of German publication DE 195 04 540 A1.

The motors 3 used are electric motors from Dynamic E Flow GmbH, Kaufbeuren, Germany, under the Capcooltech® brand, type HC and type LC.

### Reference Example 3-I

### Embodiments of Agitation Means 1.4 in Mechanochemical Mills 1

Further mechanochemical mills 1 are provided which contain further embodiments of the agitation agents 1.4 instead of the attritors 1.4.1.

3.1 The agitation means 1.4 according to FIGS. 2-1 (a), (b) and (c) are impact discs 1.4.2 each with a circular edge 1.4.2.3. The impact discs 1.4.2 each have a centrally arranged passage 1.4.2.1 for the drive shaft 3. Six equally sized impact holes 1.4.2.2 were arranged symmetrically at equal intervals around the passage 1.4.2.1. The impact discs 1.4.2 according to FIGS. 2-2 (a), (b) and (c) differ from the impact discs 1.4.2 according to FIGS. 2-1 (a), (b) and (c) only in that they have four instead of six impact holes 1.4.2.2.

3.2 The impact discs 1.4.2 according to FIGS. 3 (a), (b) and (c) have three elongated impact holes 1.4.2.2 which are bent parallel to the edge 1.4.2.3 and arranged symmetrically in a circle to one another. The impact dics 1.4.2 can be arranged on the drive shaft 2 in such a way that the impact holes 1.4.2.2 are aligned or spaced. The impact discs 1.4.2.1 can also be arranged in such a way that two or more are alternately aligned and then two or more spaced.

3.3 The impact discs 1.4.2 of FIGS. 4 (a), (b) and (c) have six symmetrically arranged, in the direction of rotation convexly curved impact webs 1.4.2.4 with a triangular profile on one of their opposite surfaces. The impact webs 1.4.2.1 each extend from the passage 1.4.2.1 to the edge 1.4.2.3. In a further embodiment, the impact webs 1.4.2.4 can be arranged on both surfaces.

3.4 The impact discs 1.4.2 of FIGS. 5 (a), (b) and (c) differ from the impact discs 1.4.2 in that the impact webs 1.4.2.4 are arranged in a straight line and have a square profile. The impact discs 1.4.2 of FIGS. 6 (a), (b) and (c) differ from those of FIGS. 5 (a), (b) and (c) only in that the impact webs 1.4.2.4 have a triangular profile. These impact discs 1.4.2 can also be arranged on the drive shaft 2 in such a way that the impact webs 1.4.2.4 are aligned or spaced. However, the impact discs 1.4.2.1 can also be arranged so that two or more are alternately aligned and then two or more spaced.

3.5 The agitation means 1.4 of FIGS. 7 (a), (b) and (c), which are arranged symmetrically to the drive shafts 2, each have two impact fans 1.4.3 radiating from a ring 1.4.3.1 surrounding the passage 1.4.2. On one of the surfaces of each of the two impact fans 1.4.3, two impact webs 1.4.2.4 with a square profile are arranged in a radial pattern.

3.6 The only difference between the impact fans 1.4.3 of FIGS. 8 (a), (b) and (c) and those of FIGS. 7 (a), (b) and (c) is that the impact webs 1.4.2.4 have a triangular profile.

3.7 The impact fans 1.4.3 of FIGS. 9 (a), (b), (c) and (d), which are arranged symmetrically to the drive shafts 3, each have a crest and trough profile 1.4.3.2 that runs parallel to the edges 1.4.2.3 and consists of two troughs and two crests.

3.8 In the agitation means 1.4 of FIGS. 10 (a), (b), (c) and (e), the ring 1.4.3.1 widens symmetrically and merges into two pairs of two radiating impact fans 1.4.3, each separated by a U-shaped gap 1.4.3.3. The four impact fans also each have a crest and trough profile 1.4.3.2 running parallel to the edges 1.4.2.3. These impact fans 1.4.3 can also be arranged on the drive shaft 2 in such a way that they are aligned or spaced. However, the impact fans 1.4.3 can also be arranged in such a way that two or more are alternately aligned and then two or more spaced.

3.9 The impact lobe 1.4.4 of FIG. 11 has three connecting webs 1.4.2.4 with a round cross-section radiating symmetrically from the ring 1.4.4.1 surrounding the drive shaft 2, to the ends of each of which a spherical impact body 1.4.4.3 is attached. The impact lobes 1.4.4 can be arranged on the drive shaft 2 in such a way that they are aligned or spaced. However, they can also be arranged in such a way that two or more are alternately aligned and then two or more are again spaced. In other embodiments, the connecting webs 1.4.2.4 can also have a quadrangular, oval or trapezoidal cross-section or a flattened cross-section as a cross-section through a knife-edge.

3.10 The flat impact blade 1.4.5 of FIG. 12 has an S-shape, in both ends 1.4.5.1 of which impact holes 1.4.2.2 are arranged. The flat impact wing 1.4.5 of FIG. 16 has a more pronounced S-shape without impact holes 1.4.2.2. The impact blades 1.4.5 can be arranged on the drive shaft 2 in such a way that they are aligned or spaced. However, they can also be arranged in such a way that two or more are alternately aligned and then two or more are again spaced.

3.11 Instead of the agitation means 1.4 and grinding media 1.2 described above, the mechanical mills 1 can also be operated with rollers 1.4.6 rotating in the opposite direction of rotation 1.4.6.1. Grinding then takes place in the roller gap. In the configuration according to FIG. 14, two parallel rollers are arranged in grinding chamber 1.1.

3.12 In the configuration according to FIG. 15, the surfaces of the two rollers 1.4.6 have interlocking teeth 1.4.6.2.

3.13 In the configuration according to FIG. 16, the roller 1.4.6 rotates against an abrasion wall 1.4.6.3, with grinding taking place in the gap between roller 1.4.6 and abrasion wall 1.4.6.3.

3.14 In the configuration of FIG. 17, the axes of rotation 1.4.6.4 of the two parallel rollers 1.4.6 intersect at an angle 1.4.6.5, resulting in additional torsion of the material to be ground F.

3.15 In the case of rollers 1.4.6 rotating in the opposite direction 1.4.6.1, these may have a springing surface 1.4.6.6. This is formed by symmetrically arranged recesses 1.4.6.7, in which springings 1.4.6.8 press the balls 1.4.6.9 out of the recesses 1.4.6.7. The grinding of the material to be ground F then takes place in the contact area of two balls 1.4.6.9. when the rollers 1.4.6 rotate. The balls 1.4.6.9 have a smaller diameter than the clear width of the recesses 1.4.6.7, so that the material to be ground F, which has entered the recesses 1.4.6.7, can trickle out again downwards when the rollers 1.4.6 are in a suitable position.

### Reference Example 4-I

### Embodiments of the Grinding Chamber 1.1 of the Mechanical Mill 1

Instead of a drum 1.5, in which the grinding chamber 1.1 has the shape of a straight cylinder, a grinding chamber 1.1 can also be used, which has at least two spherical cut-shaped grinding chambers 1.1.1 arranged one behind the other, which are formed by at least one circular constriction 1.1.2. The drive shaft 2 runs centrally through the spherical cut-shaped grinding chambers 1.1.1 and the circular constrictions 1.1.2. The dimensions of the agitation means 1.4 are adapted to the periodically changing diameter of the grinding chambers 1.1.1 (see FIG. 18). With this configuration a significantly improved mixing of the material to be ground F can be achieved.

### Examples 1-I to 17-I

### Preparation of the Activated Carbon Particles (A) According to the Invention

The mechanochemical mills 1 described in reference examples 1, 2, 3.1 to 3.15 as well as 4 are used for the preparation of the activated carbon particles (A) according to the invention.

In all cases in examples 1 to 17, coarsely particulate activated carbon particles with an average particle size d₅₀ of 5 mm are fed as material to be ground F; 1.3 under nitrogen through the respective inlet 1.5.1 into the respective grinding chamber 1.1 of the mechanical mills 1. The material to be ground F is ground at room temperature for 2 hours at 1100 rpm under nitrogen. The respective resulting activated carbon particles A according to the invention were discharged from the outlet 1.5.2 under nitrogen and analysed.

X-ray emission spectroscopic show that the activated carbon particles A are free of heavy metals. Further, GC-MS measurements show that they are free of persistent organic pollutants (POPs) and polycyclic aromatic hydrocarbons (PAHs). In the electron micrographs no hints towards meso- and macropores can be found, which is further corroborated by low internal surface areas according to BET in the range of <100 m²/g. The particle sizes determined by electron microscopy are in the range of 50 nm to 1000 nm. The activated carbon particles A are granular and have no fibrous fractions. They are of high purity, as their carbon content is 99.3 mol %.

They are therefore ideal for the preparation of decorative and medical cosmetics and care products, medical products and pharmaceuticals.

### Example Group II

### Example 1-II

Preparation and Application of a Medical Ointment for the Absorption and/or Decomposition of Chemotherapeutic Agents which are Excreted Via the Skin

An ointment for oncological use is prepared by dispersing
48.54 parts by weight of activated carbon particles A of a carbon content of 99.1 mol % and a particle size determined by electron microscopy in the range from 300 nm to 800 nm, mechanochemically pre-treated under nitrogen at 25° C., according to X-ray emission spectroscopy, free of heavy metals as well as free of persistent organic pollutants (POP), polycyclic aromatic hydrocarbons (PAH), fibrous fractions and meso- and macropores,
0.5 parts by weight of dipotassium hydrogen phosphate as buffer D and
0.05 parts by weight of Simethicone® E900 foam inhibitor D

in 52.45 parts by weight of water D under sterile conditions and carbon dioxide bubbling. The resulting pasty medicinal ointment is degassed under gentle stirring and negative pressure and filled into sterile dispensers.

In cancer patients, which have to undergo chemotherapy, very strong medicaments enter the body. These medicaments and their metabolites are partly excreted again. This happens, among other things, via the skin. This means that not only the skin of cancer patients, but also their surroundings and the nursing staff, are harmed.

The skin of cancer patients who have been treated with at least one of the following chemotherapeutic agents:
Platinum-based chemotherapeutic agents:
Carboplatin; Cisplatin
Alkylating chemotherapeutic agents based on mustard gas:
Nitrosourea derivatives:
Carmustine (BCNU)
Antimetabolites:
Methotrexate
Purine analogue metabolites
Pyrimidine analogue antimetabolites:
Fluorouracil (5-FU); Gemcitabine
Hormonal antineoplastic compounds:
Goselerin; leuprolide; temoxifen
Natural antineoplastic compounds:
Taxanes; Doclitaxel: Paclitaxel
Leukins:
Aldesleukin; interleukin-2; etoposide (VP-16); also interferon alfa; tretinoin (ATRA)
Antibiotic natural neoplastic compounds:
Bleomycin; dactinomycin; daunarubicin, doxorubicin; mitomycin
Vinca alkaloid natural antineoplasics:
Vinblastine; Vincristine
Other medications:
Daunarubicin HCl; docetaxel; doxorubicin HCl; epoetin alpha; ganciclovir sodium; gentamicin sulphate; interferon alpha; leuprolide acetate; meperidine HCl (phetidine); methadone HCl; ranitidine HCl; vinblastine sulphate; zidovudine (AZT); fluorouracil+epinephrine+bovine collagen
is treated with the medicinal ointment. After an exposure of the skin to the ointment of 10 hours, each, it is washed off. It shows that the skin of the cancer patients is not irritated or damaged and that no medicaments or metabolites can be detected on the skin any more.

### Comparative Test V1-II

Attempt to Prepare a Medicinal Ointment for the Absorption and/or Decomposition of Chemotherapeutic Agents that are Excreted Via the Skin

An attempt is made to repeat the preparation process of example 1 using activated carbon CI 77266 (carbon black) instead of activated carbon particles A. However, no stable medicinal ointment is obtained because carbon black partially floats and partially settles.

### Comparative Test V2-II

Attempt to Prepare a Medicinal Ointment for the Absorption and/or Decomposition of Chemotherapeutic Agents that are Excreted Via the Skin

An attempt is made to repeat the preparation process of example 1 with the carbon particles of an average particle size d₅₀=650±200 nm known from the German patent application DE 10 2017 010 930 A1 and the corresponding international patent application WO 2019/101357 A1 instead of the activated carbon particles A. It shows that, in contrast to the comparative test V1, a homogeneous paste is initially formed, which, however, partially segregates after prolonged storage.

### Example 2-II

### Preparation and Application of a Medicinal Sauna Paste to Detoxify the Body

To produce the medicinal sauna paste, first
200 parts by weight of activated carbon particles A with a carbon content of 99.1 mol % and a particle size in the range of 350 nm to 750 nm, mechanochemically pre-treated under argon at 10° C., according to X-ray emission spectroscopy, free of heavy metals as well as free of persistent organic pollutants (POPs), polycyclic aromatic hydrocarbons (PAHs), fibrous fractions and mesopores and macropores,
50 parts by weight of Himalayan salt D with an average particle size d₅₀ of 100 μm and
20 parts by weight of beeswax B with an average particle size of 100 μm

are mixed under sterile conditions in a powder mixer.

Under sterile conditions, a water-in-oil emulsion of
300 parts by weight of Eucerit B,
300 parts by weight of beeswax B with an average particle size of 100 μm and
100 parts by weight of water D

is prepared. The emulsion is able to take up large quantities of water.

The powder is now worked into the water-in-oil emulsion so that it is evenly dispersed in it.

To use, the sauna paste is massaged into dry or sweaty skin before, during and/or after a sauna session and left to act on the skin during the resting phase. The paste quickly absorbs the sweat and other body secretions. The sauna paste can be washed off before the next sauna session. The process can be repeated several times during a sauna visit and has a lasting detoxifying effect on the body, leaving the skin looking rejuvenated, more wrinkle-free and odour-neutralised.

### Example 3-II

### Preparation and Application of a Medicinal Peeling Paste

To produce the medical peeling paste
100 parts by weight of activated carbon particles A with a carbon content of 99.2 mol % and a particle size determined by electron microscopy in the range from 320 nm to 810 nm, mechanochemically pre-treated under nitrogen at 0° C., according to X-ray emission spectroscopy, free of heavy metals as well as free of persistent organic pollutants (POPs), polycyclic aromatic hydrocarbons (PAHs), fibrous fractions and meso- and macropores,
50 parts by weight of Himalayan salt D with an average particle size d₅₀ of 100 μm
50 parts by weight of coridone crystals with an average particle size d₅₀ of 50 μm

are mixed under sterile conditions in a powder mixer.

A water-in-oil emulsion is prepared under sterile conditions from
50 parts by weight of Eucerit B,
50 parts by weight of beeswax B with an average particle size of 100 μm and
20 parts by weight of water D.

The emulsion was able to take up large quantities of water.

The powder is now worked into the water-in-oil emulsion so that it is evenly dispersed in it.

The resulting peeling paste is massaged into the facial skin and décolleté and left to act for 30 minutes. The peeling paste is then washed off with a lukewarm cleansing solution. The pleasant effect of the peeling was clearly visible. The skin appeared fresher and smoother after the peeling. The particles A also caused a rapid uptake of waste products of the skin.

### Example 4-II

### Preparation and Application of a Fat-Free Medical Moisturising and Cleansing Cream

A fat-free moisturising and cleansing cream is prepared by dispersing
10 parts by weight of activated carbon particles A with a carbon content of 99.3 mol % and a particle size determined by electron microscopy in the range from 400 nm to 900 nm, mechanochemically pre-treated under argon at 25° C., according to X-ray emission spectroscopy, free of heavy metals as well as free of persistent organic pollutants (POPs), polycyclic aromatic hydrocarbons (PAHs), fibrous fractions and meso- and macropores,
10 parts by weight of nanocrystalline cellulose (CNC) C of an average particle size d₅₀ of 650±100 nm,
5 parts by weight of beta-glucan D and
10 parts by weight of urea D

in 150 parts by weight of water D. This forms an easily spreadable gel.

The gel is quickly taken up by the skin and keeps the skin lastingly moisturised. The particles A do not cause any discolouration of the skin due to their low dosage.

### Example 5-II

### The Preparation of a Medical Shower Gel for Cleansing and Regenerating Stressed Skin

It is well known that the skin of heavy smokers is poorly supplied with blood and therefore appears grey. In addition, the skin is contaminated by excretion products that can be traced back to the smoke.

To cleanse the skin and improve blood circulation, a shower gel made from
30 parts by weight of activated carbon particles A, mechanochemically pre-treated under argon at 25° C., free of heavy metals as well as persistent organic pollutants (POP), polycyclic aromatic hydrocarbons (PAH), fibrous fractions and meso- and macropores, with a carbon content of 99.3 mol % and a particle size in the range of 400 nm to 900 nm as determined by electron microscopy,
10 parts by weight of nanocrystalline cellulose (CNC) C of an average particle size d₅₀ of 650±100 nm,
5 parts by weight of beta-glucan D and
10 parts by weight of urea D,
40 parts by weight of cerabellina B with an average particle size d₅₀ of 50 μm,
40 parts by weight of ucerit B,
0.5 parts by weight of plant dyes and colour pigments D,
0.5 parts by weight of natural fragrances D
10 parts by weight of bentonite D,
0.5 parts by weight of a mixture of various vitamins and trace elements and
250 parts by weight of water

is prepared. The activated carbon particles A cause a rapid uptake and the removal of pollutants from the skin. Together with cerabellina and eucerit, the nanocellulose has a surface-active effect, so that a foam forms during application. The skin-permeable beta-glucan and the vitamins and trace elements help to nourish and regenerate the skin. Cerabellina and eucerit have a lubricating effect. The plant dyes and colour pigments served to tint the skin. The bentonite particles have an exfoliating effect. The fragrances lastingly mask the typical smoker's odour and the urea keeps the skin moisturised for longer.

### Example 6-II

Preparation of a Medical Protective Product for Nursing Staff Who Come into Contact with Chemotherapeutic Agents.

The cleansing/protective paste serves to protect the skin from chemicals such as chemotherapeutic agents in order to absorb them so that they cannot penetrate the skin barrier.

A paste-like ointment is produced as a protective film by dispersing
30 parts by weight of activated carbon particles A with a carbon content of 99.3 mol % and a particle size in the range of 400 nm to 900 nm as determined by electron microscopy, mechanochemically pre-treated under argon at 25° C., according to X-ray emission spectroscopy, free of heavy metals as well as free of persistent organic pollutants (POP), polycyclic aromatic hydrocarbons (PAH), fibrous fractions and meso- and macropores,
50 parts by weight of beeswax particles D with an average particle size d₅₀ of 200 μm,
2 parts by weight of nanocrystalline cellulose (CNC) C with an average particle size d₅₀ of 650±100 nm,
5 parts by weight of bentonite D with an average particle size d₅₀ of 700±50 nm,
0.5 parts by weight of dipotassium hydrogen phosphate as buffer D and
0.05 parts by weight of Simethicone® E900 foam inhibitor D

in 40.45 parts by weight of water D under sterile conditions and carbon dioxide bubbling. The resulting paste-like ointment is degassed under gentle stirring and negative pressure and filled into sterile dispensers.

In cancer patients, which have to undergo chemotherapy as described in example 1, very strong medicaments enter the body. These medicaments and their metabolites are partly excreted again. This happens, among other things, via the skin. This means that also their surroundings of the cancer patients, and in particular the nursing staff, are harmed on contact. Before and/or during the therapeutic intervention and during patient care, the hands of the nursing staff are treated with the ointment. The ointment forms a protective film that absorbs medication and metabolites. It is washed off after care and/or when care is interrupted. It shows that the skin of the nursing staff is not irritated or harmed and that no medicaments or metabolites can be detected on the skin any more.

### Example 7-II

### Provision and Topical Application of a Medicinal Ointment to Support the Treatment of Calcinosis Cutis.

Calcinosis cutis refers to deposits within the skin consisting of calcium phosphate with the formation of lime salt crystals. The resulting swellings can be accompanied by local inflammation, pain and/or plaque breaches. As a treatment, the plaques are surgically removed.

To avoid inflammation and prevent further deposits, an ointment made from
30 parts by weight of activated carbon particles A, mechanochemically pre-treated under argon at 25° C., according to X-ray emission spectroscopy, free of heavy metals as well as free of persistent organic pollutants (POPs), polycyclic aromatic hydrocarbons (PAHs), fibrous fractions and mesopores and macropores, with a carbon content of 99.2 mol % and a particle size in the range of 400 nm to 900 nm as determined by electron microscopy,
50 parts by weight of beeswax particles D with an average particle size d₅₀ of 200 μm,
2 parts by weight of nanocrystalline cellulose (CNC) C with an average particle size d₅₀ of 650±100 nm,
0.5 parts by weight of dipotassium hydrogen phosphate as buffer D,
0.05 parts by weight Simethicone® E900 foam inhibitor D,
10 parts by weight of hexahydroxycyclohexane hexaphosphoric acid ester salt nanoparticles D (sodium salt of phytic acid) with an average particle size of 650±150 nm and
1 part by weight polyethylene glycol (PEG) to open the skin pores

is dispersed in 40.0 parts by weight of water D under sterile conditions and carbon dioxide bubbling. The resulting paste-like ointment is degassed under gentle stirring and negative pressure and filled into sterile dispensers.

The ointment is applied to and around affected areas in patients with calcinosis cutis and effectively prevents inflammation and further deposits in the skin.

### Example 8-II

### Preparation and Application of a Medicinal Lotion for the Care of the Scalp and Hair Roots

To care for the scalp and hair roots, an aqueous lotion is prepared from
150 parts by weight of activated carbon particles A with a carbon content of 99.4 mol % and a particle size determined by electron microscopy in the range from 400 nm to 900 nm, which are mechanochemically pre-treated under nitrogen at 25° C. and, according to X-ray emission spectroscopy, are free of heavy metals as well as free of persistent organic pollutants (POPs), polycyclic aromatic hydrocarbons (PAHs), fibrous fractions and mesopores and macropores,
10 parts by weight of cerabellina B with an average particle size d₅₀ of 200 μm,
1 part by weight of nanocrystalline cellulose (CNC) C with an average particle size d₅₀ of 650±100 nm,
15 parts by weight of ethanol D,
5 parts by weight of panethol,
0.5 parts by weight of Cinnamomum zeylanicum leaf oil,
0.1 parts by weight polyethylene glycol (PEG) and
100 parts by weight water

under sterile conditions and bottled. To use, the medicinal lotion is massaged into the hairy scalp and left to act for 15 minutes. The lotion is then rinsed off with lukewarm water. The treatment is repeated once a day for a week. By this the hair gets visibly thicker because even smaller hairs grow stronger again due to the cleansing effect and still functional hair growth follicles are stimulated to grow hair again.

### Examples Group III

### Example 1-III

Topical Pharmaceutical According to the Invention Removal of Chemotherapeutic Agents which are Excreted Via the Skin

In a hospital pharmacy, an ointment for oncological use is prepared by dispersing
15% by weight of activated carbon particles A of a carbon content of 99.1 mol % and a particle size determined by electron microscopy in the range from 300 nm to 800 nm, mechanochemically pre-treated under nitrogen at 25° C., according to X-ray emission spectroscopy, free of heavy metals as well as free of persistent organic pollutants (POPs), polycyclic aromatic hydrocarbons (PAHs), fibrous fractions and meso- and macropores, 0.5% by weight dipotassium hydrogen phosphate as buffer D,
0.05% by weight Simethicone® E900 foam inhibitor D,
3.0% by weight magnesium aluminium silicate,
3.0% by weight citric acid crystalline,
2% by weight hydroxyacetophenone,
5.0% by weight xanthan gum with an average particle size d₅₀ of 100 μm and
10% by weight hexanediol in
61.45% by weight distilled water

under sterile conditions and carbon dioxide bubbling. The resulting medicinal ointment according to the invention is degassed under gentle stirring and negative pressure and filled into sterile dispensers. The medicinal ointment according to the invention proves to be stable in storage. Neither floating nor settling of components is observed.

In cancer patients, which have to undergo chemotherapy in hospital, very strong medicaments enter the body. These medicaments and their metabolites are partly excreted again. This happens, among other things, via the skin. This means that not only the skin of the cancer patients, but also their surroundings and the nursing staff are harmed.

Under medical supervision, twelve cancer patients are divided into three groups of four patients each. In two patients from the first group, who are treated with cisplatin, the skin in the armpits and in skin folds is treated with the medicinal ointment according to the invention. After exposure to the medicinal ointment for 10 hours in each case, it is washed off. For comparison, the skin of two patients in the group is treated with a conventional skin ointment, which is also washed off after 10 hours.

The second group of cancer patients treated with doxorubicin is treated in the same way.

The third group of cancer patients treated with vinblastine sulphate is in the same way.

Subsequently, the skin of the twelve patients is examined. It shows that the skin of the cancer patients treated with the medicinal ointment according to the invention is not irritated or damaged and that no more medicaments and metabolites can be detected on the skin.

### Comparative Test V1-III

Attempt to Prepare a Medicinal Ointment for the Absorption and/or Decomposition of Chemotherapeutic Agents which are Excreted Via the Skin

An attempt is made to repeat the preparation process of example 1 using activated carbon CI 77266 (carbon black) instead of activated carbon particles A. However, no stable medicinal ointment is obtained because carbon black partially floats and partially settles. The resulting mixture is therefore not suitable as a medicinal ointment.

### Comparative Text V2-III

Attempt to Produce a Medicinal Ointment for the Absorption and/or Decomposition of Chemotherapeutic Agents that are Excreted Via the Skin

An attempt is made to repeat the manufacturing process of example 1 with the carbon nanoparticles of an average particle size d₅₀=650±200 nm known from the German patent application DE 10 2017 010 930 A1 and the corresponding international patent application WO 2019/101357 A1 instead of the activated carbon particles A. In contrast to the comparative test V1, a homogeneous paste is initially formed, which, however, partially demixes after prolonged storage. The resulting ointment is therefore not suitable for treatment. Only when the content of carbon nanoparticles is reduced to 3% by weight is a storage-stable ointment obtained.

### Example 2-III

Topical Pharmaceutical According to the Invention for the Preventive Protection of Nursing Staff Who Come into Contact with Chemotherapeutic Agents.

The cleansing/protective paste serves as a preventive pharmaceutical to protect the skin from chemicals such as chemotherapeutic agents in order to absorb them so that they cannot penetrate the skin barrier.

A paste-like ointment is produced as a protective film by dispersing
30 parts by weight of activated carbon particles A with a carbon content of 99.3 mol % and a particle size in the range from 400 nm to 900 nm as determined by electron microscopy, mechanochemically pre-treated under argon at 25° C., according to X-ray emission spectroscopy, free of heavy metals as well as free of persistent organic pollutants (POPs), polycyclic aromatic hydrocarbons (PAHs), fibrous fractions and meso- and macropores,
50 parts by weight of beeswax particles D with an average particle size d₅₀ of 200 μm,
2 parts by weight of nanocrystalline cellulose (CNC) C with an average particle size d₅₀ of 650±100 nm,
0.5 parts by weight of dipotassium hydrogen phosphate as buffer D and
0.05 parts by weight Simethicone® E900 foam inhibitor D

in 40.45 parts by weight of water D under sterile conditions and carbon dioxide bubbling. The resulting paste-like ointment is degassed under gentle stirring and negative pressure and filled into sterile dispensers.

In cancer patients, which have to undergo chemotherapy as described in example 1, very strong medicaments enter the body. These medicaments and their metabolites are partly excreted again. This happens, among other things, via the skin. This means that also the surroundings of the cancer patients, and in particular the nursing staff, are harmed on contact. Before and/or during the therapeutic intervention and during patient care, the hands of the nursing staff are treated with the ointment. The ointment forms a protective film that absorbs medicaments and metabolites. It is washed off after care and/or when care is interrupted. It shows that the skin of the nursing staff is not irritated or harmed and that no medicaments or metabolites can be detected on the skin any more.

### Example 3a-III

### Topical Medicament According to the Invention for the Preventive Treatment of Calcinosis Cutis.

Calcinosis cutis refers to deposits within the skin consisting of calcium phosphate with the formation of lime salt crystals. The resulting swellings can be accompanied by local inflammation, pain and/or plaque breaches. As a treatment, the plaques are surgically removed.

To avoid inflammation and prevent further deposits, an ointment made from
30 parts by weight of activated carbon particles A, mechanochemically pre-treated under argon at 25° C., according to X-ray emission spectroscopy free of heavy metals as well as free of persistent organic pollutants (POPs), polycyclic aromatic hydrocarbons (PAHs), fibrous fractions and mesopores and macropores, with a carbon content of 99.2 mol % and a particle size in the range of 400 nm to 900 nm as determined by electron microscopy,
50 parts by weight of beeswax particles D with an average particle size d₅₀ of 200 μm,
2 parts by weight of nanocrystalline cellulose (CNC) C with an average particle size d₅₀ of 650±100 nm,
0.5 parts by weight of dipotassium hydrogen phosphate as buffer D,
0.05 parts by weight Simethicone® E900 foam inhibitor D,
10 parts by weight of hexahydroxycyclohexane hexaphosphoric acid ester salt nanoparticles D (sodium salt of phytic acid) with an average particle size of 650±150 nm and
1 part by weight polyethylene glycol (PEG) to open the skin pores

is dispersed in 40.0 parts by weight of water D under sterile conditions and carbon dioxide bubbling. The resulting paste-like ointment is degassed under gentle stirring and negative pressure and filled into sterile dispensers.

The ointment is applied to and around affected areas in a group of ten patients with calcinosis cutis under medical supervision and effectively prevents inflammation and further deposits in the skin.

### Example 3b-III

### Medicinal Lotion According to the Invention for the Treatment of Neurodermatitis

To alleviate the symptoms of neurodermatitis, an aqueous lotion of
150 parts by weight of activated carbon particles A with a carbon content of 99.4 mol % and a particle size determined by electron microscopy in the range from 400 nm to 900 nm, mechanochemically pre-treated under nitrogen at 25° C., according to X-ray emission spectroscopy, free of heavy metals as well as persistent organic pollutants (POPs), polycyclic aromatic hydrocarbons (PAHs), fibrous fractions and mesopores and macropores,
10 parts by weight of cerabellina with an average particle size d₅₀ of 200 μm,
1 part by weight of nanocrystalline cellulose (CNC) with an average particle size d₅₀ of 650±100 nm,
15 parts by weight of ethanol,
5 parts by weight of panethol,
0.5 parts by weight of Cinnamomum zeylanicum leaf oil,
0.1 parts by weight polyethylene glycol (PEG) and
100 parts by weight water

produced under sterile conditions and bottled. For use, the medicinal lotion is applied to the affected areas of skin on ten patients under the supervision of a dermatologist and left to act for 20 minutes. The lotion is then rinsed off with lukewarm water. The treatment is repeated once a day for a week. This eliminates the patient's symptoms for several weeks. In addition, the absence of the nagging itching means that patients no longer injure themselves by scratching the affected areas.

### Example 4-III

### Topical Pharmaceutical According to the Invention for the Treatment of Arthrosis Discomforts

Twelve patients with severe arthrosis discomforts on the hands and feet suffer chronically from intermittent, very painful swelling and deformation of the joints. Under the supervision of a rheumatologist, the patients are therefore guaranteed a medicinal ointment produced according to the method of the invention. The medicinal ointment was applied to the affected joints and limbs during acute attacks. The medicinal ointment according to the invention was prepared as follows:
8 of activated carbon particles A of a carbon content of 99.4 mol % and a particle size determined by electron microscopy in the range from 450 nm to 850 nm, mechanochemically pre-treated under argon at 25° C., according to X-ray emission spectroscopy, free of heavy metals as well as persistent organic pollutants (POPs), polycyclic aromatic hydrocarbons (PAHs), fibrous fractions and mesopores and macropores,
2 parts by weight of beeswax,
2 parts by weight of nanocrystalline cellulose (CNC) (C) with an average particle size d₅₀ of 650±100 nm,
0.5 parts by weight of dipotassium hydrogen phosphate as buffer (E) and
0.05 parts by weight of Simethicone® E900 foam inhibitor (E)

are dispersed in 52.45 parts by weight of distilled water under sterile conditions and carbon dioxide bubbling. The resulting paste-like ointment is degassed under gentle stirring and negative pressure and filled into sterile dispensers. The paste-like ointment has a bluish to purplish grey colour. When massaged in, the ointment according to the invention was absorbed into the skin together with the activated carbon particles A and left no black residue on the skin.

In all patients, the swelling and pain quickly subsided within 10 to 20 minutes. The success of the treatment lasted up to four days in all patients.

### Example 5-III

### Topical Pharmaceutical According to the Invention for the Treatment of Nail Fungus and Nail Bed Inflammation

The medicinal ointment according to the invention of example 1 is used under medical supervision for the treatment of nail fungus and nail bed inflammation in six patients. In one case, the infestation of the toenails was so severe that the toenails were deformed and bent upwards due to spongy, foul-smelling excretions and deposits under the nails. Repeated treatment with the medicinal ointment according to the invention eliminates the nail fungus and the nail bed inflammations and, in particular, the excretions and deposits under the nails disappear in the one patient.

### Example 6-III

Topical Pharmaceutical According to the Invention for the Treatment of Allergic Skin Reactions Caused by Contact with Oak Processionary Moths

Twenty-nine test subjects under medical supervision by an allergologist are contacted in a controlled manner with the hairs of oak processionary moths in a grid pattern on the inside of their right forearms and their right wrists. The allergic reactions occurred immediately, with the symptoms, itching and pain being described as mild by two test subjects and severe by the others. The exposed areas were treated in the morning, at midday and in the evening with the topical pharmaceutical according to the invention of example 1. In twenty-eight test test subjects, the allergic reactions disappeared permanently. One test subject does not respond to the treatment.

### Particular Aspects Part I

Aspect I-1. Activated carbon particles A having a carbon content >99 mol % and a particle size determined by electron microscopy in the range from 100 nm to 1000 nm, which are mechanochemically pre-treated under at least one inert gas and, according to X-ray emission spectroscopy, are free of heavy metals as well as free of persistent organic pollutants (POPs), polycyclic aromatic hydrocarbons (PAHs), fibrous fractions and meso- and macropores.

Aspect I-2. Aactivated carbon particles A according to aspect I-1, characterised in that the at least one inert gas is selected from the group consisting of nitrogen, helium, neon, argon and xenon.

Aspect I-3. Activated carbon particle A according to aspect I-1 or I-2, characterised in that the absence of meso- and macropores is determined by electron microscopy.

Aspect I-4. Mechanochemical process for the preparation of activated carbon particles (A) according to one of aspects I-1 to I-3, characterised in that one
(I) heavy-metal-free, PAH- and/or POP-containing, coarsely particulate activated carbon particles are provided as material to be ground (F),
(II) the material to be ground (F) are fed continuously or discontinuously under inert gas into at least one grinding chamber (1.1) of at least one mechanical mill (1) lined with at least one heavy-metal-free, inorganic, technical ceramic, and
(III) grinds therein with grinding media (1.2) moved by agitation means (1.4) or with rollers (1.4.6), the surfaces of which are coated with at least one heavy-metal-free, inorganic, technical ceramic, at a constant and/or variable rotational speed under inert gas until particle sizes in the range from 100 nm to 1000 nm are reached, after which
(IV) the activated carbon particles (A) are discharged under inert gas.

Aspect I-5. Mechanochemical process according to aspect I-4, characterised in that in each case at least one heavy-metal-free, hard, inert oxide, nitride and/or carbide is added as a particulate grinding aid.

Aspect I-6. Mechanochemical process according to aspect I-5, characterised in that the at least one particulate grinding aid is selected from the group consisting of quartz, glass, aluminium nitride, silicon nitride, silicon carbide, silicon carbide nitride, aluminium oxide and boron nitride.

Aspect I-7. Mechanochemical process according to aspect I-4 or I-5, characterised in that the activated carbon particles (A) are separated from the at least one particulate grinding aid via the difference in density.

Aspect I-8. Mechanochemical process according to one of aspects I-4 to I-7, characterised in that the activated carbon particles (A) are sifted or classified according to their particle size.

Aspect I-9. Mechanochemical process according to one of aspects I-4 to I-8, characterised in that the grinding of the at least one material to be ground (F) or of the mixture of at least one material to be ground (F) and at least one grinding aid is carried out at a temperature of the grinding media (1.2) and of the at least one material to be ground F of −273° C. to +500° C.

Aspect I-10. Mechanochemical process according to one of apects I-4 to I-9, characterised in that a mechanical mill (1) is used for this purpose, which comprises at least one rotatable or stationary mechanochemical reactor (1.1) containing a rotatable or stationary drum (1.5) with a grinding chamber (1.1) with at least one inlet (1.5.1) for the material to be ground (F; 1.3), at least one outlet (1.5.2) for the activated carbon particles (A) or the mixture of activated carbon particles (A) and the at least one grinding aid as well as a plurality of fixed or rotatable agitation means (1.4), wherein
the drum (1.5) of the rotatable mechanochemical reactor and waveguide (1.1) has a disc-shaped vertical drum wall (1.5.3) which is connected at its centre to a rotatable drive shaft (2) which can be driven by a motor (3), and
the drum (1.5) of the stationary mechanochemical reactor and waveguide (1.1) has, by means of a drive shaft (2), rotatable agitation means (1.4) for mixing the grinding media (1.2) and the material to be ground (F; 1.3) or rotatable rollers (1.4.6) aligned in the longitudinal direction of the drum (1.5), wherein the drive shaft (2) is driven by a motor (3) and is guided by the disc-shaped vertical drum wall (1.5.3) through the passage (1.5.3.1),
the agitation means (1.4) are selected from the group consisting of impact discs (1.4.2), impact fans (1.4.3), impact lobes (1.4.4) and impact blades (1.4.5), which have the impact holes (1.4.2.2), impact webs (1.4.2.4), crest and trough profiles (1.4.3.2), connecting webs (1.4.4.2) and impact bodies (1.4.4.3) arranged symmetrically to the drive shaft (2), and wherein
the rotatable rollers (1.4.6) are rotatable relative to one another in opposite directions of rotation (1.4.6.1) and their axes of rotation (1.4.6.4) are parallel to one another or have an angle of inclination (1.4.6.5) or are rotatable against an abrasion surface (1.4.6.3),
wherein all surfaces inside the mechanical mill (1) which come into contact with the material to be ground (F) or with the at least one material to be ground (F) and the at least one grinding aid are coated with at least one heavy-metal-free, inorganic, technical ceramic.

Aspect I-11. Mechanochemical process according to aspect I-10, characterised in that the heavy-metal-free, inorganic, technical ceramic is selected from the group consisting of aluminium oxide, boron carbide, boron nitride, boron nitride carbide, calcium silicate, silicon oxide, silicon carbide, silicon nitride, silicon oxide nitride, silicon oxide carbide, silicon nitride carbide, silicon oxide nitride carbide and glass ceramics.

Aspect I-12. Mechanochemical process according to aspect I-10 or I-11, characterised in that the mill (1) comprises an agitation means (1.4) of the same type and/or at least two different types of agitation means (1.4), wherein the agitation means (1.4), selected from the group consisting of impact discs (1.4.2), impact fans (1.4.3), impact lobes (1.4.4) and impact blades (1.4.5), which have impact holes (1.4.2.2), impact webs (1.4.2.4), crest and trough profiles (1.4.3.2), connecting webs (1.4.4.2) and impact bodies (1.4.4.3), which are arranged symmetrically to the drive shaft (3), are aligned and/or spaced in the direction of the drive shaft (2).

Aspect I-13. Mechanochemical process according to one of the aspects I-10 to I-12, characterised in that the grinding chamber (1.1) has at least two spherical cut-shaped grinding chambers (1.1.1) arranged one behind the other, which are formed by at least one circular constriction (1.1.2), wherein drive shaft (2) extends centrally through the spherical cut-shaped grinding chambers (1.1.1) and the circular constrictions (1.1.2) and the dimensions of the agitation means (1.4) are adapted to the periodically changing diameter of the grinding chambers (1.1.1).

Aspect I-14. Mechanochemical process according to any one of aspects I-4 to I-13, characterised in that the process is carried out in a cascade of mechanical mills (1).

Aspect I-15. Use of the activated carbon (A) according to any one of Aspects I-1 to I-3 or the activated carbon (A) produced by the mechanochemical process according to any one of aspects I-4 to I-14 for the preparation of decorative and medical cosmetics and care products, medical products and pharmaceuticals.

Aspect I-16. Use of the activated carbon (A) according to aspect I-15, characterised in that the decorative and medical cosmetics and care products, the medical products and the pharmaceuticals are powders, solids, gels, creams, ointments, lotions, drops, sprays, metered-dose aerosols, gargle solutions, lozenges, enemas, clysters, foams and release materials as well as coatings on textile fabrics, face masks, gauze, plasters, foams, toiletries, envelopes, bandages, tampons and medical products.

Aspect I-17. Use of the activated carbon (A) according to aspect I-16, characterised in that the medical products are for the care of overlappings of skin on the chest, abdomen and groin, of mucous membranes, eyes, ears, mouth, lips, fingernails, toenails, cornea, warts and scars, the removal and/or neutralisation of excretions of noxae, drugs, toxins, medication, acids, bases and odorous substances via the skin, the alleviation of unpleasant and/or painful accompanying symptoms and side effects of lesions, itching, intertrigo, burning, infections, cancer therapy, chemotherapy, chondrocalcinosis, psoriasis, neurodermatitis, acne, eczema and atrophic eczema, warts, rosacea, herpes, shingles, measles, mumps, rubella, chickenpox, hand-mouth-foot diseases, hand and foot syndromes, redness, rheumatism and arthrosis as well as preventive protection and support of healing processes in the case of existing infections, wounds or burns.

### Particular Aspects Part II

Aspect II-1. Heavy-metal-free topical medical products and cosmetics, containing activated carbon particles (A) with a carbon content >99 mol % and a particle size determined by electron microscopy in the range from 100 nm to 1000 nm, which are mechanochemically pre-treated under at least one inert gas and are, according to X-ray emission spectroscopy, free of heavy metals as well as free of persistent organic pollutants (POPs), polycyclic aromatic hydrocarbons (PAHs), fibrous fractions and meso- and macropores.

Aspect II-2. Heavy metal-free topical medical products and cosmetics according to aspect II-1, characterised in that the at least one inert gas is selected from the group consisting of nitrogen, helium, neon, argon and xenon.

Aspect II-3. Heavy metal-free topical medical products and cosmetics according to aspect II-1 or II-2, characterised in that the absence of meso- and macropores is determined by electron microscopy.

Aspect II-4. Heavy-metal-free topical medical products and cosmetics according to one of aspects II-1 to II-3, characterised in that the topical medical products and cosmetics contain the activated carbon particles (A), based on the respective total amount of a topical medical product, in an amount of from 0.1% by weight to 50% by weight.

Aspect II-5: Heavy-metal-free topical medical products and cosmetics according to one of aspects II-1 to II-5, characterised in that they additionally contain
(B) particles of an average particle size d₅₀ of from 10 nm to <1000 μm, comprising at least one type and/or consisting of at least one type of nanocrystalline and/or microcrystalline natural waxes and/or semisynthetic waxes based on natural waxes and/or biodegradable microplastics.

Aspect II-6: Heavy-metal-free topical medical products and cosmetics according to aspect II-5, characterised in that the particles (B) can be produced from natural and semi-synthetic waxes using the GAS process (Gas Antisolvent Recrystallization), the PCA process (Precipitation with a Compressed Fluid Antisolvent), the PGSS process (Particles from Gas Saturated Solutions) or the RESS process (Rapid Expansion of Supercritical Solutions).

Aspect II-7: Heavy-metal-free topical medical products and cosmetics according to one of aspects II-1 to II-6, characterised in that they additionally contain
(C) particles having an average particle size d₅₀ of from 10 nm to <1000 nm, comprising at least one type and/or consisting of at least one type of nanocrystalline, nanofibrillar, microcrystalline and/or microfibrillar polysaccharides.

Aspect II-8. Heavy metal-free topical medical products and cosmetics according to aspect II-7, characterised in that the particles (C) are selected from the group consisting of cellulose nanofibres (CNF), microfibrillar celluloses (MFC), microcrystalline celluloses (MCC), nanofibrillar polysaccharides (NFP), nanocrystalline celluloses (CNC) and bacterial nanocelluloses (BNC), waste from mashes from papermaking and fibres, as well as particles from used textiles.

Aspect II-9. Heavy metal-free topical medical products and cosmetics according to aspect II-7 or II-8, characterised in that the particles (C) have an average particle size d₅₀=650±200 nm.

Aspect II-10 Heavy-metal-free topical medical products and cosmetics according to any one of aspects II-1 to II-9, characterised in that they additionally comprise
(D) at least one ingredient selected from the group consisting of water, layered silicates, milled clays, bolus americus clays, healing clays, nutshells, willow barks, lapacho healing barks, silicas, pumice stones, geopolymers, ambers, gemstones, trace elements and neutral inorganic salts, beta-glucans, glutathione, albumin, xylan, phytates, complexing agents, antioxidants, radical scavengers, menthol, caffeine, teein, papain, esculin semihydrate, carotenoids, lecithins, abscisic acid, cyanidin, guanidin, urea, thiourea, fillers, solvents, moisturisers, solubilisers, non-ionic wetting agents, buffers, thickeners, binders, coating agents, disintegration accelerators, disintegrants, anti-friction agents, lubricants, mould release agents, flow regulators, antioxidants, preservatives, sweeteners, flavour corrigents, absorption accelerators, alkalising agents, acidifying agents, neutralising agents, foam inhibitors and defoamers, dyes, colour pigments, oxidising agents, reducing agents, stabilisers, propellants, opacifiers, pearlescent agents, denaturants, plasticisers, minerals, vitamins, micelles, superabsorbents, refatting substances, oils, fragrances, flavourings, perfumes, moisturisers, moisturising agents, active ingredients for skin care, deodorising agents, film-forming agents, gelling agents, tannins, tanning agents, tanning extracts, bitter substances, plant extracts, honeys, colostrum, cytostatics, antibodies, immunoglobulins, essential and non-essential amino acids and fats, hyaluronic acid, aloe vera gel, liposomes, alkylene glycols, polyalkylene ether glycols, proteins, peptides, carbohydrates, lipids, nucleic acids, nucleic acid fragments, antiviral compounds, anti-inflammatory compounds, antibiotics, cell differentiation reagents, analgesics, drugs, anaesthetics, contrast agents, enzymes, cytokines, antihistamines, immunomodulators, haemostatic reagents, hormones, angiogenic and antiangiogenic agents, neurotransmitters, therapeutic oligonucleotides, virus particles, growth factors, retinoids, cell adhesion factors, extracellular matrix glycoproteins, osteogenic factors, antibodies, antigens, steroids, analgesics, potassium sulphate, royal jelly, propolis, encapsulated fragrances, nutrients, hexahydroxycyclohexane hexaphosphoric acid ester salt nanoparticles and surfactants.

Aspect II-11. Heavy metal-free topical medical products and cosmetics according to any one of aspects II-1 to II-10, characterised in that they are in the form of powders, solids, gels, creams, ointments, lotions, drops, sprays, metered dose aerosols, gargle solutions, lozenges, enemas, clysters, foams and release materials as well as as coatings on textile fabrics, face masks, gauze, plasters, foams, toilet utensils, envelopes, bandages, tampons and on and in medical devices and roll-on sticks.

Aspect II-12. Heavy metal-free topical medical products and cosmetics according to any one of aspects II-1 to II-12, characterised in that their pH is between 3 and 7.

Aspect II-13. Method for the preparation of topical medical products and cosmetics according to any one of aspects II-1 to II-12, characterised in that
(I) at least one type of activated carbon particles (A) is mixed with at least one type of particles (B) and/or at least one type of particles (C) and the resulting mixture is homogenised, and
(II) the resulting homogenised mixture (AB), (AC) or (ABC) is processed into powders, solids, granules, gels, creams, ointments, lotions, drops, sprays, metered-dose aerosols, gargle solutions, lozenges, enemas, clysters, suppositories or foams and/or applied to and/or in textile fabrics, face masks, gauze, compresses, bandages, tampons, plasters, medical products and/or release materials.

Aspect II-14. process according to aspect II-13, characterised in that the activated carbon particles (A) are mixed with at least one type of particles (B) and/or at least one type of particles (C) and/or at least one ingredient (D) and the resulting mixture (ABC), (ABD), (ACD) or (ABCD) is homogenised.

Aspect II-15. Use of the heavy metal-free topical medical products and cosmetics according to any one of aspects II-1 to II-12 or the heavy metal-free topical medical products and cosmetics produced according to the method according to aspect II-13 or II-14 for the care of overlapping skin on the breast, abdomen and groin, of mucous membranes, eyes, ears, mouth, lips, fingernails, toenails, cornea, warts and scars, the removal and/or rendering harmless of excretions of noxae, drugs, toxins, medicaments, acids, bases and odorous substances via the skin, the alleviation of unpleasant and/or painful accompanying symptoms and side effects of lesions, itching, intertrigo, burning, infections, for the accompanying support of cancer therapy and chemotherapy as well as the treatment of chondrocalcinosis, psoriasis, neurodermatitis, acne, eczema, atrophic eczema, warts, rosacea, herpes, shingles, measles, mumps, rubella, chickenpox, hand-mouth-foot diseases, hand and foot syndromes, redness, rheumatism and arthrosis as well as preventive protection and support of healing processes in the case of existing infections, wounds or burns.

### Particular Aspects Part III

Aspect III-1. Activated carbon particles (A) with a carbon content >99 mol % and a particle size determined by electron microscopy in the range from 100 nm to 1000 nm, which are mechanochemically pre-treated under at least one inert gas and are, according to X-ray emission spectroscopy, free of heavy metals as well as free of persistent organic pollutants (POPs), polycyclic aromatic hydrocarbons (PAHs), fibrous fractions and meso- and macropores, for use in medicine

Aspect III-2. Heavy-metal-free topical pharmaceuticals containing activated carbon particles (A) which are mechanochemically pre-treated under at least one inert gas, are, according to X-ray emission spectroscopy, free from heavy metals as well as free from persistent organic pollutants (POPs), polycyclic aromatic hydrocarbons (PAHs), fibrous fractions and mesopores and macropores, have a carbon content >99 mol % and a particle size determined by electron microscopy in the range from 100 nm to 1000 nm, for use in medicine.

Aspect III-3. Heavy metal-free topical pharmaceuticals for use in medicine according to aspect III-2 for the elimination of chemotherapeutic agents excreted via the skin, for the preventive treatment of calcinosis cutis and for the treatment of neurodermatitis, of arthrosis complaints, of nail fungus to nail bed inflammation and of allergic reactions to oak processionary moths.

Aspect III-4. Heavy metal-free topical pharmaceuticals for use in medicine according to aspect III-2 or III-3, characterised in that the at least one inert gas is selected from the group consisting of nitrogen, helium, neon, argon and xenon.

Aspect III-5. Heavy metal-free topical medicaments for use in medicine according to any one of aspects III-2 to III-4, characterised in that the absence of meso- and macropores is determined by electron microscopy.

Aspect III-6. Heavy metal-free topical pharmaceuticals for use in medicine according to any one of aspects III-2 to III-5, characterised in that the specific internal surface area of the activated carbon particles (A) according to BET is <200 m²/g.

Aspect III-7: Heavy-metal-free topical pharmaceuticals for use in medicine according to any one of aspects III-2 to III-6, characterised in that they do not contain organically bound halogens.

Aspect III-8. Heavy metal-free topical pharmaceuticals for use in medicine according to any one of aspects III-2 to III-7, characterised in that the topical pharmaceuticals contain the activated carbon particles (A), based on the respective total amount of a topical pharmaceutical, in an amount of from 0.1% by weight to 50% by weight.

Aspect III-9. Heavy metal-free topical pharmaceuticals for use in medicine according to one of aspects III-2 to III-8, characterised in that they additionally contain
(B) particles of an average particle size d₅₀ from 10 nm to <1000 μm, comprising at least one type and/or consisting of at least one type of nanocrystalline and/or microcrystalline natural waxes and/or semisynthetic waxes based on natural waxes and/or biodegradable microplastics.

Aspect III-10. Heavy metal-free topical pharmaceuticals for use in medicine according to aspect III-6, characterised in that the particles (B) can be produced from natural and semi-synthetic waxes using the GAS process (Gas Antisolvent Recrystallization), the PCA process (Precipitation with a Compressed Fluid Antisolvent), the PGSS process (Particles from Gas Saturated Solutions) or the RESS process (Rapid Expansion of Supercritical Solutions).

Aspect III-11. Heavy metal-free topical pharmaceuticals for use in medicine according to any one of aspects III-1 to III-10, characterised in that they additionally contain
(C) particles of an average particle size d₅₀ of from 10 nm to <1000 nm, comprising at least one type and/or consisting of at least one type of nanocrystalline, nanofibrillar, microcrystalline and/or microfibrillar polysaccharides.

Aspect III-12. Heavy metal-free topical pharmaceuticals for use in medicine according to aspect III-11, characterised in that the particles (C) are selected from the group consisting of cellulose nanofibres (CNF), microfibrillar celluloses (MFC), microcrystalline celluloses (MCC), nanofibrillar polysaccharides (NFP), nanocrystalline celluloses (CNC) and bacterial nanocelluloses (BNC), waste from papermaking mashes and fibres, as well as particles from used textiles.

Aspect III-13. Heavy metal-free topical pharmaceuticals for use in medicine according to aspect III-11 or III-12, characterised in that the particles (C) have an average particle size d₅₀=650±200 nm.

Aspect III-14. Heavy metal-free topical pharmaceuticals for use in medicine according to any one of aspects III-2 to III-13, characterised in that they additionally comprise
(D) at least one ingredient selected from the group consisting of water, layered silicates, milled clays, bolus americus clays, healing clays, nutshells, willow barks, lapacho healing barks, silicas, pumice stones, geopolymers, ambers, gemstones, trace elements and neutral inorganic salts, beta-glucans, glutathione, albumin, xylan, phytates, complexing agents, antioxidants, radical scavengers, menthol, caffeine, teein, papain, esculin semihydrate, carotenoids, lecithins, abscisic acid, cyanidin, guanidin, urea, thiourea, fillers, solvents, moisturisers, solubilisers, non-ionic wetting agents, buffers, thickeners, binders, coating agents, disintegration accelerators, disintegrants, anti-friction agents, lubricants, mould release agents, flow regulators, antioxidants, preservatives, sweeteners, flavour corrigents, absorption accelerators, alkalising agents, acidifying agents, neutralising agents, foam inhibitors and defoamers, dyes, colour pigments, oxidising agents, reducing agents, stabilisers, propellants, opacifiers, pearlescent agents, denaturants, plasticisers, minerals, vitamins, micelles, superabsorbents, refatting substances, oils, fragrances, flavourings, perfumes, moisturisers, moisturising agents, active ingredients for skin care, deodorising agents, film-forming agents, gelling agents, tannins, tanning agents, tanning extracts, bitter substances, plant extracts, honeys, colostrum, cytostatics, antibodies, immunoglobulins, essential and non-essential amino acids and fats, hyaluronic acid, aloe vera gel, liposomes, alkylene glycols, polyalkylene ether glycols, proteins, peptides, carbohydrates, lipids, nucleic acids, nucleic acid fragments, antiviral compounds, anti-inflammatory compounds, antibiotics, cell differentiation reagents, analgesics, drugs, anaesthetics, contrast agents, enzymes, cytokines, antihistamines, immunomodulators, haemostatic reagents, hormones, angiogenic and antiangiogenic agents, neurotransmitters, therapeutic oligonucleotides, virus particles, growth factors, retinoids, cell adhesion factors, extracellular matrix glycoproteins, osteogenic factors, antibodies, antigens, steroids, analgesics, potassium sulphate, royal jelly, propolis, encapsulated fragrances, nutrients, hexahydroxycyclohexane hexaphosphoric acid ester salt nanoparticles and surfactants.

Aspect III-15. Heavy metal-free topical pharmaceuticals for use in medicine according to any one of aspects III-2 to III-14, characterised in that they are in the form of powders, solids, gels, creams, ointments, lotions, drops, sprays, metered dose aerosols, gargle solutions, lozenges, enemas, clysters, foams and release materials as well as as coatings on textile fabrics, face masks, gauze, plasters, foams, toilet utensils, envelopes, pads, tampons and on and in medical devices and roll-on sticks.

Aspect III-16. Heavy metal-free topical pharmaceuticals for use in medicine according to any one of aspects III-2 to III-15, characterised in that their pH is between 3 and 7.

Aspect III-17. Method for the preparation of topical pharmaceuticals for use in medicine according to any one of aspects III-2 to III-16, characterised in that
(I) at least one type of activated carbon particles (A) is mixed with at least one type of particles (B) and/or at least one type of particles (C) and the resulting mixture is homogenised, and
(II) the resulting homogenised mixture (AB), (AC) or (ABC) is processed into powders, solids, granules, gels, creams, ointments, lotions, drops, sprays, metered-dose aerosols, gargle solutions, lozenges, enemas, clysters, suppositories or foams and/or applied to and/or in textile fabrics, face masks, gauze, compresses, bandages, tampons, plasters, medical devices and/or release materials.

Aspect III-18. Method according to aspect III-17, characterised in that the activated carbon particles (A) are mixed with at least one type of particles (B) and/or at least one type of particles (C) and/or at least one ingredient (D) and the resulting mixture (ABC), (ABD), (ACD) or (ABCD) is homogenised.

Aspect III-19. Striking preparations of the topical pharmaceuticals for use in medicine according to any one of aspects III-1 to III-15 in the form of powders, solids, gels, creams, ointments, lotions, drops, sprays, metered dose aerosols, gargles, lozenges, enemas, clysters, foams, release materials coatings and coating materials can be prepared on and/or in release materials, sponges, textile fabrics, tampons, face masks, gauze and plasters as well as on and/or in roll-on pens, dispensers and medical devices.

1-17. (canceled)

18. Activated carbon particles (A) having a carbon content >99 mol % and a particle size determined by electron microscopy in the range from 50 nm to 1000 nm or from 100 nm to 1000 nm, which are mechanochemically pre-treated under at least one inert gas and are, according to X-ray emission spectroscopy, free of heavy metals as well as free of persistent organic pollutants (POP), polycyclic aromatic hydrocarbons (PAH), fibrous fractions and mesopores and macropores.

19. A mechanochemical process for the preparation of activated carbon particles (A) according to claim 18, wherein:
(I) heavy-metal-free, PAH- and/or POP-containing, coarsely particulate activated carbon particles are provided as material to be ground (F),
(II) the material to be ground (F) is fed continuously or discontinuously under inert gas into at least one grinding chamber of at least one mechanical mill lined with at least one heavy-metal-free, inorganic, technical ceramic, and
(III) it is ground therein with grinding media moved by agitation means or with rollers, the surfaces of which are coated with at least one heavy-metal-free, inorganic, technical ceramic, at a constant and/or variable rotational speed under inert gas until particle sizes in the range from 50 nm to 1000 nm or from 100 nm to 1000 nm are reached, after which
(IV) the activated carbon particles (A) are discharged under inert gas.

20. The mechanochemical process according to claim 19, wherein in each case at least one heavy-metal-free, hard, inert oxide, nitride and/or carbide is added as a particulate grinding aid, wherein the at least one particulate grinding aid is preferably selected from the group consisting of quartz, glass, aluminium nitride, silicon nitride, silicon carbide, silicon carbide nitride, aluminium oxide and boron nitride.

21. The mechanochemical process according to claim 19, wherein the activated carbon particles (A) are separated from the at least one particulate grinding aid by means of the difference in density, and/or in that the activated carbon particles (A) are sifted or classified according to their particle size.

22. The mechanochemical process according to claim 19, wherein a mechanical mill is used for this purpose, which comprises at least one rotatable or stationary mechanochemical reactor, containing a rotatable or stationary drum with a grinding chamber with at least one inlet for the material to be ground (F), at least one outlet for the activated carbon particles (A) or the mixture of activated carbon particles (A) and the at least one grinding aid as well as a plurality of fixed or rotatable agitation means, wherein
the drum of the rotatable mechanochemical reactor and waveguide has a disc-shaped vertical drum wall which is connected at its centre to a rotatable drive shaft which can be driven by a motor, and
the drum of the stationary mechanochemical reactor and waveguide has, by means of a drive shaft, rotatable agitation means for mixing the grinding media and the material to be ground (F) or rotatable rollers aligned in the longitudinal direction of the drum, wherein the drive shaft is driven by a motor and is guided by the disc-shaped vertical drum wall through a passage,
the agitation means are selected from the group consisting of impact discs, impact fans, impact lobes and impact blades, which have impact holes, impact webs, crest and trough profiles, connecting webs and impact bodies that are arranged symmetrically to the drive shaft, and wherein
the rotatable rollers are rotatable relative to one another in opposite directions of rotation and their axes of rotation are parallel to one another or have an angle of inclination or are rotatable against an abrasion surface,
wherein all surfaces inside the mechanical mill which come into contact with the material to be ground (F) or with the at least one material to be ground (F) and the at least one grinding aid are coated with at least one heavy metal-free, inorganic, technical ceramic, preferably selected from the group consisting of aluminium oxide, boron carbide, boron nitride, boron nitride carbide, calcium silicate, silicon oxide, silicon carbide, silicon nitride, silicon oxide nitride, silicon oxide carbide, silicon nitride carbide, silicon oxide nitride carbide and glass ceramics.

23. The mechanochemical process according to claim 22, wherein the mill has an agitation means of the same type and/or at least two different types of agitation means, wherein the agitation means are aligned and/or spaced in the direction of the drive shaft.

24. The mechanochemical process according claim 22, wherein the grinding chamber has at least two spherical cut-shaped grinding chambers arranged one behind the other, which are formed by at least one circular constriction, wherein the drive shaft extends centrally through the spherical cut-shaped grinding chambers and the circular constrictions and the dimensions of the agitation means are adapted to the periodically changing diameter of the grinding chambers.

25. The mechanochemical process according to claim 19, wherein the process is carried out in a cascade of mechanical mills.

26. A method of using the activated carbon (A) according to claim 18 for the preparation of decorative and medical cosmetics and care products, medical products and pharmaceuticals, for example in the form of lipid-free formulated creams.

27. The method of using the activated carbon (A) according to claim 26, wherein:
the decorative and medical cosmetics and care products, the medical products and the pharmaceuticals are powders, solids, gels, creams, ointments, lotions, drops, sprays, metered dose aerosols, gargle solutions, lozenges, enemas, clysters, foams and release materials as well as coatings on textile fabrics, face masks, gauze, plasters, foams, toilet utensils, compresses, bandages, tampons and medical devices,
whereby the medical products are preferably used for the care of overlapping skin on the chest, abdomen and groin, of mucous membranes, eyes, ears, mouth, lips, fingernails, toenails, corneas, warts and scars, the removal and/or rendering harmless of excretions of noxae, drugs, toxins, medicines, acids, bases and odorous substances via the skin, the alleviation of unpleasant and/or painful accompanying symptoms and side effects of lesions, itching, intertrigo, burning, infections, cancer therapy, chemotherapy, chondrocalcinosis, psoriasis, neurodermatitis, acne, eczema and atrophic eczema, for warts, rosacea, herpes, shingles, measles, mumps, rubella, chickenpox, hand-mouth-foot diseases, hand and foot syndromes, redness, rheumatism and arthrosis as well as for the preventive protection and support of healing processes in existing infections, wounds or burns, preferably as creams, particularly preferably as lipid-free formulated creams.

28. Heavy metal-free topical pharmaceuticals, medical products and cosmetics containing activated carbon particles (A) according to claim 18.

29. The heavy metal-free topical pharmaceuticals, medical products and cosmetics according to claim 28, wherein the topical medical products and cosmetics contain the activated carbon particles (A) in an amount of 0.1% by weight to 50% by weight, based on the respective total amount of a topical pharmaceutical, medical product or cosmetic.

30. The heavy-metal-free topical pharmaceuticals, medical products and cosmetics according to claim 28, further comprising:
(B) particles of an average particle size d₅₀ of 10 nm to <1000 m, containing at least one type and/or consisting of at least one type of nanocrystalline and/or microcrystalline natural waxes and/or semisynthetic waxes based on natural waxes and/or biodegradable microplastics,
wherein the particles (B) are preferably producible from natural and semi-synthetic waxes by means of the GAS process (Gas Antisolvent Recrystallisation), the PCA process (Precipitation with a Compressed Fluid Antisolvent), the PGSS process (Particles from Gas Saturated Solutions) or the RESS process (Rapid Expansion of Supercritical Solutions),
and/or
that they additionally contain
(C) particles of an average particle size d₅₀ of 10 nm to <1000 nm, containing at least one type and/or consisting of at least one type of nanocrystalline, nanofibrillar, microcrystalline and/or microfibrillar polysaccharides,
wherein the particles (C) are preferably selected from the group consisting of cellulose nanofibres (CNF), microfibrillar celluloses (MFC), microcrystalline celluloses (MCC), nanofibrillar polysaccharides (NFP), nanocrystalline celluloses (CNC) and bacterial nanocelluloses (BNC), waste from paper mashes from paper preparation and fibres, as well as particles from used textiles, and/or have an average particle size d₅₀=650±200 nm,
and/or
that they additionally contain
(D) at least one ingredient selected from the group consisting of water, layered silicates, milled clays, bolus americus clays, healing clays, nutshells, willow bark, lapacho bark, silica, pumice stones, geopolymers, amber, gemstones, trace elements and neutral inorganic salts, beta-glucans, glutathione, albumin, xylan, phytates, complexing agents, antioxidants, radical scavengers, menthol, caffeine, teein, papain, esculin semihydrate, carotenoids, lecithins, abscisic acid, cyanidin, guanidin, urea, thiourea, fillers, solvents, moisturisers, solubilisers, non-ionic wetting agents, buffers, thickeners, binders, coating agents, disintegration accelerators, disintegrants, anti-friction agents, lubricants, mould release agents, flow regulators, antioxidants, preservatives, sweeteners, flavour corrigents, absorption accelerators, alkalising agents, acidifying agents, neutralising agents, foam inhibitors and defoamers, dyes, colour pigments, oxidising agents, reducing agents, stabilisers, propellants, opacifiers, pearlescent agents, denaturants, plasticisers, minerals, vitamins, micelles, superabsorbers, refatting substances, oils, fragrances, flavourings, perfumes, moisturisers, moisturising agents, active ingredients for skin care, deodorising agents, film-forming agents, gelling agents, tannins, tanning agents, tanning extracts, bitter substances, plant extracts, honeys, colostrum, cytostatics, antibodies, immunoglobulins, essential and non-essential amino acids and fats, hyaluronic acid, aloe vera gel, liposomes, alkylene glycols, polyalkylene ether glycols, proteins, peptides, carbohydrates, lipids, nucleic acids, nucleic acid fragments, antiviral compounds, anti-inflammatory compounds, antibiotics, cell differentiation reagents, analgesics, drugs, anaesthetics, contrast agents, enzymes, cytokines, antihistamines, immunomodulators, haemostatic reagents, hormones, angiogenic and antiangiogenic agents, neurotransmitters, therapeutic oligonucleotides, viral particles growth factors, retinoids, cell adhesion factors, extracellular matrix glycoproteins, osteogenic factors, antibodies, antigens, steroids, analgesics, potassium sulphate, royal jelly, propolis, encapsulated fragrances, nutrients, hexahydroxycyclohexane hexaphosphoric acid ester salt nanoparticles and surfactants.

31. The heavy metal-free topical pharmaceuticals, medical products and cosmetics according to claim 28, wherein:
the topical pharmaceuticals, medical products and cosmetics are in the form of powders, solids, gels, creams, ointments, lotions, drops, sprays, metered dose aerosols, gargle solutions, lozenges, enemas, clysters, foams and release materials, as well as coatings on textile fabrics, face masks, gauze, plasters, foams, toilet utensils, compresses, bandages, tampons and on and in medical devices and roll-on sticks, preferably as creams, particularly preferably as lipid-free formulated creams.

32. The heavy metal-free topical pharmaceuticals, medical products and cosmetics according to claim 28, wherein the topical pharmaceuticals, medical products and cosmetics has a pH value between 3 and 7.

33. A method for the preparation of heavy-metal-free topical pharmaceuticals, medical products and cosmetics containing activated carbon particles (A), according to claim 30, wherein:
(I) at least one type of activated carbon particles (A) is mixed with at least one type of particles (B) and/or at least one type of particles (C) and the resulting mixture is homogenised and
(II) the resulting homogenised mixture (AB), (AC) or (ABC) is processed into powders, solids, granules, gels, creams, ointments, lotions, drops, sprays, metered-dose aerosols, gargle solutions, lozenges, enemas, clysters, suppositories or foams and/or applied to and/or into textile fabrics, face masks, gauze, compresses, bandages, tampons, plasters, medical devices and/or release materials, wherein the activated carbon particles (A) are preferably mixed with at least one type of particles (B) and/or at least one type of particles (C) and/or at least one ingredient (D) and the resulting mixture (ABC), (ABD), (ACD) or (ABCD) is homogenised,
wherein the topical pharmaceuticals, medical products and cosmetics are preferably processed into creams, particularly preferably lipid-free formulated creams.

34. The use of the heavy-metal-free topical pharmaceuticals, medical products and cosmetics containing activated carbon particles (A) according to claim 28, wherein the use is:
for the care of overlapping skin on the chest, abdomen and groin, of mucous membranes, eyes, ears, mouth, lips, fingernails, toenails, cornea, warts and scars, the removal and/or rendering harmless of excretions of noxae, drugs, toxins, medicaments, acids, bases and odorous substances via the skin, the alleviation of unpleasant and/or painful accompanying symptoms and side effects of lesions, itching, intertrigo, burning, infections, for the accompanying support of cancer therapy and chemotherapy as well as the treatment of chondrocalcinosis, psoriasis, neurodermatitis, acne, eczema, atrophic eczema, warts, rosacea, herpes, shingles, measles, mumps, rubella, chickenpox, hand-mouth-foot diseases, hand and foot syndromes, redness, rheumatism and arthrosis as well as the preventive protection and support of healing processes in the case of existing infections, wounds or burns, or
for the removal of chemotherapeutic agents that are excreted via the skin, for the preventive treatment of calcinosis cutis and for the treatment of neurodermatitis, arthrosis complaints, nail fungus, nail bed inflammation and allergic reactions to oak processionary moths.
